(19) United States
(12) Patent Application Publication
(10) Pub. No.: US 2016/0213628 A1
(43) Pub. Date: Jul. 28, 2016

(54) DETERRING ABUSE OF PHARMACEUTICAL PRODUCTS AND ALCOHOL

(71) Applicant: NOVA SOUTHEASTERN UNIVERSITY, Fort Lauderdale, FL (US)

(72) Inventors: Hossein Omidian, Fort Lauderdale, FL (US); David J. Mastropietro, Fort Lauderdale, FL (US); Srinath Muppalaneni, Fort Lauderdale, FL (US)

(21) Appl. No.: 14/917,368

(22) PCT Filed: Sep. 9, 2014
(86) PCT No.: PCT/US14/54863

§ 371 Date: Mar. 8, 2016

Related U.S. Application Data
(60) Provisional application No. 61/875,173, filed on Sep. 9, 2013.
(60) Provisional application No. 61/919,443, filed on Dec. 20, 2013.
(60) Provisional application No. 61/918,870, filed on Dec. 20, 2013.
(60) Provisional application No. 61/918,890, filed on Dec. 20, 2013.
(60) Provisional application No. 61/918,879, filed on Dec. 20, 2013.

(51) Int. Cl. A61K 31/135; A61K 47/10; A61K 47/48; A61K 47/32; A61K 9/14; A61K 9/20

(52) U.S. Cl. CPC A61K 31/135; A61K 9/143; A61K 9/2009; A61K 47/4823; A61K 47/48015; A61K 47/32; A61K 47/10

(57) ABSTRACT

A therapeutic dosage form includes a pharmaceutically active ingredient, a crosslinked polyacid, and a linear polyacids. The crosslinked polyacid is insoluble in water, and the linear polyacid is soluble in water. The linear polyacid possesses sufficient binding sites to form a stable complex with the pharmaceutically active ingredient. The ingredients are formed into a tablet or capsule, either admixed, in layers, or separated by a coating. Abuse is deterred in that crushing causes the active ingredient to be bound and not abusable, and placing the dosage in solution causes a strong complex to be formed between the polyacid and the active ingredient, including a solution with ethanol. Other therapeutic dosage forms for reducing the incidence of tampering and abuse of pharmaceutical products and alcohol, and specifically preventing the isolation and concentration of drug constituents for misuse, and preventing excessive intake are also disclosed.

### CROSS REFERENCE TO RELATED APPLICATIONS

This application claims the benefit of related U.S. Patent Application 61/875,173 filed Sep. 9, 2013, entitled “ABUSE-DETERRENT PHARMACEUTICAL COMPOSITIONS”; U.S. Patent Application 61/918,870, filed Dec. 20, 2013, entitled “ABUSE DETERRENTS IN PHARMACEUTICAL COMPOSITIONS”; U.S. Patent Application 61/918,879 filed Dec. 20, 2013, entitled “A THERAPEUTIC COMPOSITIONS FOR ALCOHOL CESSATION AND ABUSE”; U.S. Patent Application 61/918,890, filed Dec. 20, 2013, entitled “POWERFUL DETERRENT AGENTS FOR ABUSABLE MEDICATIONS”; and U.S. Patent 61/919,443, filed Dec. 20, 2013, entitled “AVERSIVE SUPERDETERRENT AGENT FOR ABUSABLE MEDICATIONS”, the contents of each of which are incorporated herein by reference in their entirety.

### FIELD OF THE DISCLOSURE

The disclosure relates to reducing the incidence of tampering and abuse of pharmaceutical products and alcohol, and more particularly preventing the isolation and concentration of drug constituents for misuse, and preventing excessive intake.

### BACKGROUND OF THE DISCLOSURE

Prescription drug abuse is at epidemic proportions, and has become a serious problem affecting public health. Pain medications, CNS depressants and stimulants are among those commonly abused via different techniques including snorting, injection, and co-ingestion with alcohol.

Tablets, transdermal patches, and nasal sprays are the most commonly abused pharmaceutical products and are frequently tampered by crushing and/or mixing with water and alcohol. The initial step of crushing is needed to abuse drugs by almost all routes such as snorting, injecting, smoking, and orally to achieve rapid absorption of the entire dose at once. It is also very common for abusers to take crushed drug products with alcoholic drinks or other beverages to heighten the effects of the drug and allow quicker entry into the bloodstream.

Abuse of prescription drugs is now a fastest-growing drug problem in the US. In almost 10 years, the number of Americans abusing controlled prescription drugs rose from 7.8 million in 1992 to 15.1 million in 2003 [1]. This high number of abusers represents more people than the combined total of those abusing cocaine, hallucinogens, inhalants, and heroin. Recent results from the 2009 National Survey on Drug Use and Health [2] report that an average of 7,000 people each day experiment for the first time with a prescription pain medication, tranquilizer, stimulant, or sedative. The large increase in prescribing and abuse of prescription medications has affected public health in many ways. The number of emergency room visits and unintentional deaths due to controlled prescription drugs has increased sharply over the century from 1998 to 2008 [3]. Although these medications are generally safe to take as prescribed, they can be deadly when abused, or and taken inappropriately.

Attributed largely to the misuse and abuse of prescription medications, drug poisonings and overdoses now kill more Americans than car accidents for the first time in history [4]. The prescription pain medications have been most responsible for these deaths; as the number of drug poisoning deaths involving such medications has risen from 4,000 in 1999 to 14,800 in 2008, representing over 40% of drug poisoning deaths in 2008 [4].

As more Americans began abusing prescription drugs, so has the number seeking treatment. Every year, from 1999 to 2008, there has been an increase in the number of individuals seeking treatment for opioid prescription pain medications [5]. Along with the increased abuse and treatment of prescription drugs comes rising medical costs. The overall direct cost to health insurers resulting from the nonmedical use of prescription painkillers has been estimated up to $72.5 billion annually [6].

The abuse and misuse of prescription medications is not limited to the United States. According to the United Nations 2011 World Drug Report [7], the demand for cocaine, heroin, and cannabis (each an illicit drug) has declined or stayed the same while the production and abuse of prescription opioid pain medications has grown. There are many factors contributing to this widespread abuse. One incentive type factor is the perception that prescription medications are safe and associated with a low potential for harm and abuse compared to illicit drugs. Another factor is the ease of obtaining prescription medications. Many abusers find that prescription medications are much easier to obtain than illicit (street) drugs. A national survey [8] showed that over 70% of people who abused prescription pain medications obtained them directly from friends or relatives, while only 4.3% acquiring them from drug dealers or strangers.

Even though young adults are those most likely to abuse prescription drugs, young adolescent children and older adults abused them too. The abuse of pain medications among adolescents has increased from 3.3% in 1992 to 9.5% in 2004, and stayed close to this level through 2010 [9]. Those aged 50 to 59 also showed an increase in abuse from 2.7% in 2002 to 6.2% in 2009 [2]. Serious health risks are associated with abuse of these medications in patients over fifty. The number of emergency room visits involving the misuse and abuse of prescription drugs in those aged over 50 increased 121.1% from 2004 to 2008 [10].

The most commonly prescribed medications by physicians are oral tablets and capsules, and they have become the most commonly abused medications. The National Institute on Drug Abuse lists the top three drug classes abused as opioids, central nervous system (CNS) depressants, and stimulants [11]. Opioids are medications similar to morphine (e.g., oxycodone, hydrocodone, codeine), which commonly produce a sense of well-being or euphoria in the abuser. CNS depressants are medications typically used for sleep or anxiety disorders, which cause drowsiness and a calming effect in users. Stimulants are drugs commonly referred to as “uppers”, because they produce alertness and energy with an overall elevation in mood that makes them top candidate drugs for abuse.

When an oral drug no longer gives the same high or euphoric feeling, abusers may take more (overdose), take it in a different way, or manipulate the medication to produce a greater or more rapid euphoria [13]. Altering the medication from its original form for this purpose can be defined as tampering. Tampering typically results in the drug being absorbed at a faster rate or allows the medication to be given by another route. The most common methods of tampering are as follows:

crushing a tablet medication into a powder so that it can be inhaled through the nose and rapidly enter the bloodstream;

once a tablet medication is reduced to small particles by crushing or chewing, it may be taken orally, smoked, snorted, or mixed with a solution and injected for faster results; and

when swallowed with medications, alcohol causes certain drugs to dissolve more quickly and to be absorbed rapidly, which dangerously intensifies the drug's effect on the body [14].

One approach to address the foregoing is Reformulated Oxycontin® (a powerful pain medication). The original Oxycontin tablet was meant to deliver the drug slowly over 12 hours, but abusers quickly found the effect of alcohol in enhancing the drug solubility and that chewing or crushing the tablet could defeat the slow release mechanism [15]. In response, the manufacturer reformulated the product into a similar looking tablet, resistant to crushing into small pieces, forming a thick viscous fluid upon contact with liquids.

REMOXY is a capsule type product containing thick “taffy” like material inside the capsule shell, which purports to slow down drug release. As of this writing, FDA approval has been delayed due to product inconsistency and unpredictable performance [17].

Embeda® was approved in the U.S. in 2009, and is a capsule that contains small beads of morphine and a segregated compartment which releases a drug upon crushing that stops morphine from working [18]. In 2011, the product was voluntarily recalled for stability reasons and has yet to return to the marketplace. Reformulated Opana ER (oxymorphone HCl) utilizes a melt extrusion or a thermal process. Exalgo (Hydromorphone) has a hard exterior shell and gelling agent. Oxecta (oxycodone HCl) contains gelling agent and a nasal irritant. Nucynta ER (Tapentadol) uses an approach similar to the reformulated Opana ER

Tampering methods such as crushing, chewing, grating, or grinding a dosage form to obtain smaller particles allows the drug to be taken by alternate routes, and speeds the rate of dissolution. For example, crushing a tablet would allow the abuser to snort or smoke the product, or mix with a suitable liquid to dissolve the drug and inject the resultant solution parenterally after filtration. A great concern to public health is when abusers tamper with extend-release formulations containing a large amount of drug meant to be absorbed slowly over several hours. The ability to easily destroy the controlled release mechanisms of these formulations by crushing or other means allows high levels of drug to be absorbed rapidly and to dangerous levels in the user. Tampering of this nature can occur intentionally as in the case of an abuser seeking to get high, or unintentionally by a legitimate user crushing the tablet for ease of swallowing. Drugs and other excipients soluble in ethanol also have the added danger of “dose-dumping”, meaning release of the entire drug load at once, when taken with an alcoholic beverage.

A great concern to public health is when abusers tamper with extend-release formulations containing a large amount of drug meant to be absorbed slowly over several hours. The ability to easily destroy the controlled release mechanisms of these formulations by crushing or other means allows high levels of drug to be absorbed rapidly and to dangerous levels in the user. Tampering of this nature can occur intentionally as in the case of an abuser seeking to get high, or unintentionally by a legitimate user crushing the tablet for ease of swallowing. Drugs and other excipients soluble in ethanol also have the added danger of “dose-dumping”, meaning release of the entire drug load at once, when taken with an alcoholic beverage.

The development of dosage forms intended to deter, discourage and prevent the nonmedical use of highly abused drugs was initially made popular by the incorporation of narcotic antagonist into tablet formulations prone to parenteral abuse. Most of these formulations pertain to oral dosage forms, particularly solid dosage forms. First attempts were the use of opioid antagonist that were not orally bioavailable, but would exert their effect if the dosage form was injected by parenteral routes. In the late 1970's, a combination of the prescription drug pentazocine (Talwin®) along with the antihistamine tripelennamine were being used together parenterally to gain a high similar to heroin [40]. To combat this problem, naloxone was included into the formulation, and marketed in the United States as Talwin®Nx. The naloxone in the reformulated tablet was sufficient to antagonize the effects of pentazocine when administered parenterally yet have limited effects when taken orally. The addition of naloxone to tablets was therefore included to deter intravenous abuse. More recently in 2002, the FDA approved the combination of buprenorphine with naloxone (Suboxone®) as a sublingual tablet for the treatment of opioid dependence outside of a clinic. The naloxone component is added to help deter misuse such as parenteral injection during maintenance therapy. Concerns such as the slow dissolution of the sublingual tablets and unintentional child exposures led to the development of oral films with better mucoadhesion and oral dissolution [41].

U.S. Pat. No. 7,968,119 describes compositions consisting of an opioid agonist together with a sequestered antagonist agent and an antagonist removal system [42]. U.S. Pat. No. 4,457,933 describes combining the analgesic dose of an opioid with a specific low ratio of naloxone. U.S. Pat. No. 6,228,863 [43] describes oral dosage forms that makes extracting an opioid analgesic from the combined agonist/antagonist mixture at least a two-step process. U.S. Pat. No. 6,696,088 [44], U.S. Pat. No. 7,658,939 [45], U.S. Pat. No. 7,718,192 [46], U.S. Pat. No. 7,842,309 [47], and U.S. Pat. No. 7,842,311 [48] describe tamper-resistant oral dosage forms having a sequestered antagonist. U.S. Pat. No. 7,914,818 [49] describes both a non-releasable sequestered opioid antagonist along with a releasable opioid antagonist together with the opioid agonist.

U.S. Pat. No. 3,980,766 [50] describes adding ingestible solid materials that have rapid thickening properties in water. Compositions containing aqueous gelling agents are described in U.S. Pat. No. 4,070,494 [51]. U.S. Pat. No. 6,309,668 describes tablet compositions having two or more layers, where the gelling agent is in a separate layer from the drug [52]. Abuse deterrent dosage forms containing a gel forming polymer along with an analgesic opioid, nasal tissue irritant, and emetic or inert emesis causing agent are described in U.S. Pat. No. 7,201,920 [53], U.S. Pat. No. 7,476,402 [54], and U.S. Pat. No. 7,510,726 [55]. Other patents having deterrent agents include U.S. Pat. No. 4,175,119 describing the use of emetic coating, and U.S. Pat. No. 4,459,278 describing binding the emetic agents to an inert substance [57].

Consumption of alcohol is a major public health concern associated with significant costs and high rates of mortality. Three oral medications, i.e. disulfiram (Antabuse®), naltrexone (Depade®, ReVia®) and acamprosate (Campral®) are currently approved to treat alcohol dependence. In addition, an injectable form of naltrexone (Vivitrol®) is also available.

Carbonaceous adsorbents can be modified to produce micro-porous structures giving the material an extremely large surface area. Activated charcoal is an example of carbonaceous material that first undergoes carbonization, and then an activation step to produce a highly porous material capable of adsorption. Activation refers to the development of surface area by increasing pore volume, pore diameter, and porosity of the material through a physical, chemical, or physiochemical activation process [63]. The activation process usually occurs at high temperatures in an environment of an activating gas (e.g. carbon dioxide, steam) or a chemical activating agent (e.g., phosphoric acid, zinc chloride) or both. The raw material to make activated carbon may start from a variety of sources including animal (animal charcoal), natural gas incomplete combustion (e.g., gas black, furnace black), and burning of fats and oils (e.g., lamp black). However, activated charcoal is derived from wood or vegetable origins [64].

Activated charcoal is a black porous material that is insoluble in water and organic solvents. Commercially, it is available in many forms such as granular, extruded, pelletized or powdered in varying particle sizes. Activated charcoal for medicinal purposes must meet compendial or similar standards (BP, USP), which includes testing to demonstrate its adsorption power. Additionally, it should have a surface area of at least 900 m²/g to have adequate adsorption potential [65]. The properties of activated charcoal are due largely to its enormous surface area and surface chemistry. The average surface area range of activated charcoal is between 800-1,200 m²/g, and may be modified to as large as 2,800-3,500 m²/g [66]. Although the exact mechanisms of interaction between activated carbon and a substrate are complex, adsorption processes are the most well studied, and may be chemical or physical in nature [64]. For the adsorption process in a liquid, activated charcoal acts as the insoluble adsorbent to which a water soluble adsorbate is adsorbed onto. Adsorption may be dependent on polarity, ionization, and environmental pH, with organic and large poorly water soluble materials adsorbing to a higher degree than polar small molecules [66]. Orally, activated charcoal is most notably used as a gastrointestinal decontamination agent to treat acute overdoses and poisonings [71].

Prescription drug abuse is now a widespread phenomenon, particularly regarding opioid narcotic analgesics. These medications are having alarming effects to public health as the rate of their abuse increases. According to the CDC, drug overdose deaths in the United States have continuously increased for 11 consecutive years in 2010 with opioids being the driving factor and prescription drugs as a whole involved in 60% of cases [74]. Other abusable analgesics such as TRAMADOL have also increased. For example, visits to the emergency room from Tramadol overdoses which cause seizures and repository or CNS depression in patients have recently increased [75]. The use of activated charcoal to treat Tramadol overdose was investigated in-vitro and in-vivo, and reported to bound up to 0.05 mg of Tramadol for each mg of activated charcoal [76].

### SUMMARY OF THE DISCLOSURE

In an embodiment of the disclosure, a therapeutic dosage form comprises one or more pharmaceutically active ingredients; one or more crosslinked polyacids; and one or more linear polyacids.

In various embodiments thereof, the dosage form further includes at least one of tablet excipients for tableting, capsule excipients for encapsulation, and patch excipients for transdermal patches; the pharmaceutically active ingredients treats an illness selected from the group consisting: anxiety, depression, sleep disorders, pain, lack of energy, attention deficit, cough, and cold; the one or more pharmaceutically active ingredients is selected from the group of barbiturates comprising: phenobarbitals, benzodiazepines, codeine, morphine, oxycodone, oxymorphone, hydrocodone, hydromorphone, Tramadol, amphetamines, methyl phenidate, dextromethorphan, and pseudoephedrine; the one or more pharmaceutically active ingredients is in the form of its weak base; the dosage form is a tablet; the dosage form is a capsule; and/or the dosage has a form selected from the group consisting of gel, suppository, suspension, emulsion, micro sized dispersion, nano-sized dispersion, semi-solid, paste, ointment, lozenge, strip, film, and rod.

In additional embodiments thereof, the weak base is selected from the group consisting a salt of: organic acids, inorganic acids, hydrochloric acid, hydrosulfuric acid, hydrophosphoric acid, and tartaric acid; the crosslinked polyacid is insoluble in water; the crosslinked polyacid is made using at least one internal hydrolytic process, irradiative process, thermal process, addition of a bi-chemical crosslinker, addition of polyfunctional chemical crosslinker; the crosslinked polyacid possess sufficient binding sites to form a stable complex with the one or more pharmaceutically active ingredients; and/or the crosslinked polyacid is selected from the group consisting of sodium carboxymethylcellulose, sodium carboxymethylstarch, alginic acid salt, polyacrylate salt, polymethacrylate salt, poly(potassium sulfopropyl acrylate), poly(2-acrylamido 2-methyl1-propane sulfonic acid (AMPS).

In yet further embodiments thereof; the polyacid is at least one of internally crosslinked or chemically crosslinked; the salt is one of sodium, potassium, and ammonium; the dosage form comprises one or more crosslinked polyacids, at a polyacid to pharmaceutically active ingredient weight ratio of about 0.1 to about 500, and advantageously about 1 to about 50; the one or more linear polyacids is soluble in water; the linear polyacid possesses sufficient binding sites to form a stable complex with the one or more pharmaceutically active ingredients; the linear polyacid is selected from the group of water soluble polymers comprising salts of: carboxymethylcellulose, carboxymethylstarch, alginic acid, polyacrylic acid, polymethacrylic acid, poly(sulfopropyl acrylate), and poly(2-acrylamido 2-methyl1-propane sulfonic acid (AMPS); the salts are mono-valent; the salt is one of sodium, potassium, and ammonium; the one or more linear polyacids is sodium carboxymethylcellulose; and/or the dosage comprises 1-99 wt % of the one or more linear polyacids.

In still further embodiments thereof, the one or more pharmaceutically active ingredients, one or more crosslinked polyacids, and one or more linear polyacids are compressed into a tablet along with other tablet excipients; the one or more pharmaceutically active ingredients is a weak acid supplied as a salt; and/or the dosage form further includes at least one of a crosslinked polybase and a linear polybase.

In other embodiments thereof, the dosage form further includes one or more tablet excipients, and wherein a tablet is formed by: mixing an aqueous solution of the one or more pharmaceutically active ingredients, the one or more linear polymers, and the one or more crosslinked polyacids; drying the mix; and compressing the dried mix along with the one or more tablet excipients.

In another embodiment of the disclosure, a therapeutic dosage form comprises one or more pharmaceutically active ingredients; one or more inorganic clays (a) with binding sites sufficient to form a stable complex with the one or more pharmaceutically active ingredients, when the clay is exposed to the one or more pharmaceutically active ingredients when the dosage form is crushed or subjected to non-physiological tampering conditions, and (b) the clay is physically separated from contact with the one or more pharmaceutically active ingredients before the dosage is orally administered.

In various embodiments thereof, at least one of tablet excipients for tableting, capsule excipients for encapsulation, and patch excipients for transdermal patches; the one or more pharmaceutically active ingredients treats an illness selected from the group consisting: anxiety, depression, sleep disorders, pain, lack of energy, attention deficit, cough, and cold; the one or more pharmaceutically active ingredients is selected from the group of barbiturates comprising: phenobarbitals, benzodiazepines, codeine, morphine, oxycodone, oxymorphone, hydrocodone, hydromorphone, Tramadol, amphetamines, methyl phenidate, dextromethorphan, and pseudoephedrine; the one or more pharmaceutically active ingredients is in the form of its weak base; the dosage form is a tablet; the dosage form is a capsule; and/or the dosage has a form selected from the group consisting of gel, suppository, suspension, emulsion, micro sized dispersion, nano-sized dispersion, semi-solid, paste, ointment, lozenge, strip, film, and rod.

In other embodiments thereof, the clay is coated with a coating agent to physically separate the clay from contact with the one or more pharmaceutically active ingredients before the dosage is administered; the clay is coated with a water-insoluble coating material; the inorganic clay is selected from the group consisting: phyllosilicates; halloysite; kaolinite; illite; montmorillonite; vermiculite; talc; palygorskite; pyrophyllite; zeolite; zeolite made of aluminum silicate sheets; zeolite made of aluminum silicate sheets containing other cations; and/or the inorganic clay is bentonite; the clay is an aggregate produced using at least one of conventional wet granulation and hot melt extrusion techniques.

In other embodiments thereof, the clay is an aggregate including at least one of a water-soluble or water-dispersible polymer selected from one or more of the group consisting of synthetic polymer, polyacrylic acid, polyvinyl alcohol, polyethylene glycol, polyvinyl pyrrolidone, polyethylene oxide, hydrocolloid gum, alginic acid and its salts, chitosan, carrageenan, gum Arabic, guar gum, agar agar, gelatin, xanthan, locust bean gum, cellulosic, methyl cellulose, carboxymethyl cellulose, hydroxyethyl cellulose, hydroxypropyl cellulose, hydroxypropyl methylcellulose, starch; the clay is an aggregate including a polymer, the aggregate bound with hydroxypropyl methylcellulose; and/or the coating agent is selected from one or more of the group consisting of water-insoluble polymer, ethyl cellulose, cellulose acetate, cellulose acetate phthalate, cellulose acetate butyrate, shellac, methacrylate copolymer, acrylate copolymer, enteric acrylate copolymer, non-enteric acrylate copolymer, poly(lactic acid), poly(lactide-co-glycolide), hydroxypropyl methylcellulose phthalate, hydroxypropyl methylcellulose acetate succinate, polyvinyl acetate.

In still further embodiments thereof, the coating agent is a methacrylic acid ethyl acrylate copolymer; one of the solid or the dispersion form of methacrylic acid ethyl acrylate copolymer is used; the coating agent is selected from one or more of the group consisting of: animal wax, beeswax, plant wax, carnauba wax, petroleum wax, paraffin, polyethylene wax, stearic acid, magnesium stearate; the clay has the form of particles or aggregates, and the dosage form comprises clay particles or aggregates to pharmaceutically active ingredient weight ratio of about 0.1 to about 500, and advantageously about 1 to about 50; the clay has the form of coated particles or aggregates, and the one or more pharmaceutically active ingredients and coated clay are mixed and compressed into a tablet; the dosage form is a tablet formed as a plurality of layers, wherein the clay is in a different layer than the one or more pharmaceutically active ingredient; the clay has the form of coated particles or aggregates, and is coated in a continuous extrusion process; and/or the dosage form is a capsule, and wherein the one or more pharmaceutically active ingredient is wet granulated, and then incorporated into the capsule along with the coated clay.

In another embodiment of the disclosure, a therapeutic dosage form comprises one or more pharmaceutically active ingredients, and at least one of activated carbon or activated porous non-carbon material adsorbent to the one or more pharmaceutically active ingredients and having sufficient adsorption sites to accommodate substantially all of the one or more pharmaceutically active ingredients; and a physical separation between the at least one of activated carbon or activated porous non-carbon material and the one or more pharmaceutically active ingredients within the dosage form, the at least one of activated carbon or activated porous non-carbon material contactable with the one or more pharmaceutically active ingredients to adsorb the one or more pharmaceutically active ingredients when the physical separation is removed prior to administration of the dosage form.

In various embodiments thereof, the dosage form further including at least one of tablet excipients for tableting, capsule excipients for encapsulation, and patch excipients for transdermal patches; the one or more pharmaceutically active ingredients treats an illness selected from the group consisting: anxiety, depression, sleep disorders, pain, lack of energy, attention deficit, cough, and cold; the one or more pharmaceutically active ingredients is selected from the group of barbiturates comprising: phenobarbitals, benzodiazepines, codeine, morphine, oxycodone, oxymorphone, hydrocodone, hydromorphone, Tramadol, amphetamines, methyl phenidate, dextromethorphan, and pseudoephedrine; the one or more pharmaceutically active ingredients is in the form of its weak base; the dosage form is a tablet; the dosage form is a capsule; and/or the dosage has a form selected from the group consisting of gel, suppository, suspension, emulsion, micro sized dispersion, nano-sized dispersion, semi-solid, paste, ointment, lozenge, strip, film, and rod.

In further embodiments thereof, the physical separation is a coating about the at least one of activated carbon or activated porous non-carbon material; the coating is polymeric; the at least one of activated carbon or activated porous non-carbon material is modified via grafting to another substrate configured to enhance an adsorption property of the at least one of activated carbon or activated non-carbon material; the substrate enhances the adsorption by at least one of chemical or mechanical interaction with the at least one of activated carbon or activated porous non-carbon material; the activated carbon material is at least one of an activated charcoal or medicinal carbon; at least one of activated carbon or activated porous non-carbon material has the form of fine particles or aggregates; the at least one of activated carbon or activated porous non-carbon material is coated with a water-insoluble coating material; the activated porous non-carbon material is an activated silica or activated alumina.

In other embodiments thereof, the at least one of activated carbon or activated porous non-carbon material are produced as aggregates using at least one of conventional wet granulation or hot melt extrusion techniques; the at least one of activated carbon or activated porous non-carbon material is formed as an aggregate using a binder selected from the group consisting of at least one of: water-soluble polymer, water-dispersible polymer, synthetic polymer, polyacrylic acid, polyvinyl alcohol, polyethylene glycol, polyvinyl pyrrolidone, polyethylene oxide, hydrocolloid gum, alginic acid and its salts, chitosan, carrageenan, gum Arabic, guar gum, agar agar, gelatin, xanthan, locust bean gum, cellulosic material, methyl cellulose, carboxymethyl cellulose, hydroxyethyl cellulose, hydroxypropyl cellulose, hydroxypropyl methylcellulose, and starch; a binder for making the aggregate is hydroxypropyl methylcellulose.

In yet further embodiments thereof, the particles or aggregates are coated with a material selected from the group consisting of at least one of: water-insoluble polymer, ethyl cellulose, cellulose acetate, cellulose acetate phthalate, cellulose acetate butyrate, shellac, methacrylate copolymer, acrylate copolymer, poly(lactic acid), poly(lactide-co-glycolide), hydroxypropyl methylcellulose phthalate, hydroxypropyl methylcellulose acetate succinate, and polyvinyl acetate; the coating is methacrylic acid ethyl acrylate copolymer; at least one of the solid or the dispersion form of the methacrylic acid ethyl acrylate copolymer is used; the coating is selected from a group consisting of at least one of: animal wax, beeswax, plant wax, carnauba wax, petroleum wax, paraffin, polyethylene wax, water-insoluble wax, stearic acid, and magnesium stearate.

In additional embodiments thereof, the at least one of activated carbon or activated porous non-carbon material comprises 1-99 wt % of the dosage form; the at least one of activated carbon or activated porous non-carbon material is formed and the one or more pharmaceutically active ingredients are physically mixed and compressed into a tablet along with other tablet excipients; the dosage form is a multi-layer tablet, wherein the at least one of activated carbon or activated porous non-carbon material is separated from the drug layer within the tablet.

In another embodiment thereof, the one or more pharmaceutically active ingredients is wet granulated; the at least one of activated carbon or activated porous non-carbon material is wet granulated separately from the wet granulated pharmaceutically active ingredients; the wet granulated activated carbon or activated porous non-carbon material is coated with a water insoluble material; and the wet granulated pharmaceutically active ingredients and the wet granulated and coated activated carbon or activated porous non-carbon material are incorporated into a capsule.

In another embodiment of the disclosure, a therapeutic dosage form comprises one or more pharmaceutically active ingredients; one or more organic binding agents; one or more inorganic binding agents; and one or more adsorbents.

In various embodiments thereof, the one or more organic binding agent is capable of binding to positively charged pharmaceutically active ingredients; the one or more organic binding agent is at least one crosslinked anionic hydrophilic polymer; the at least one crosslinked anionic hydrophilic polymer is crosslinked carboxymethylcellulose; the one or more organic binding agent is used at a concentration greater than 60% to maximize trapping of the one or more pharmaceutically active ingredients in water, saline, and hydroalcohols, while allowing release of the one or more pharmaceutically active ingredients in 0.1N HCl; the one or more organic binding agent is used at 100% concentration to maximum release of the one or more pharmaceutically active ingredients in 0.1N HCl; the one or more inorganic binding agent is capable of binding to positively charged pharmaceutically active ingredients; the one or more inorganic binding agent is a clay material; the clay material is calcium or sodium bentonite; the clay material is used at a concentration between about 50% and about 100% to maximum trapping of the one or more pharmaceutically active ingredients in water, saline, aqueous ethyl alcohol, and acidic solutions; and/or the clay material is used at 100% concentration to maximum trapping of the one or more pharmaceutically active ingredients in hydroalcoholic solutions.

In yet further embodiments thereof, the one or more adsorbents has a porous structure capable of adsorbing the one or more pharmaceutically active ingredients; the one or more adsorbents is silica or charcoal; the one or more adsorbents is medicinal charcoal; the one or more adsorbents is used at a concentration between about 0% and about 80% to maximum trapping of the one or more pharmaceutically active ingredients in water, saline, and hydroalcohols but allows release of the one or more pharmaceutically active ingredients in 0.1N HCl; the one or more adsorbents is used at 100% concentration to maximum trapping of the one or more pharmaceutically active ingredients in 0.1N HCl; the one or more pharmaceutically active ingredients is trapped from solution in water, saline, hydroalcoholic solutions, and acidic solutions; and/or the one or more pharmaceutically active ingredients is trapped from solution in water, saline, EtOH 40%, and a pH3 solution, but is released in 0.1N HCl.

In other embodiments thereof, the one or more organic binding agents is crosslinked sodium carboxymethylcellulose; the one or more inorganic binding agents is bentonite; and the one or more adsorbents is charcoal; at least one of crosslinked sodium carboxymethylcellulose, bentonite, and charcoal is coated; each of crosslinked sodium carboxymethylcellulose, bentonite, and charcoal is coated; and/or none of crosslinked sodium carboxymethylcellulose, bentonite, and charcoal are coated.

In further variations thereof (where AcDiSol alternatively represents a crosslinked sodium carboxymethylcellulose):

the dosage form is configured to actively trap the one or more active ingredients from its solution in water, in saline, in EtOH 40% and in a pH3 solution, however it releases the active ingredient in 0.1N HCl solution;

the dosage form includes AcDiSol, Bentonite, and medicinal Charcoal;

the dosage form includes 0-100% AcDiSol (or crosslinked sodium carboxymethylcellulose).

the dosage form includes 0-100% Bentonite.

the dosage form includes 0-100% Charcoal.

the dosage form includes 70% Bentonite and 30% Charcoal if only water used to extract the active;

the dosage form includes 100% Bentonite if only EtOH used to extract the active;

the dosage form includes 23% Bentonite and 77% Charcoal if only saline used to extract the active;

the dosage form includes 10% AcDiSol, 50% Bentonite and 40% Charcoal if only pH 3 solution used to extract the active;

the dosage form includes 100% Bentonite or 100% Charcoal if only 0.1N HCl used to extract the active;

the dosage form includes 100% Bentonite if water and EtOH used to extract the active;

the dosage form includes 60% Bentonite and 40% Charcoal if water and saline used to extract the active;

the dosage form includes 70% Bentonite and 30% Charcoal if water and a pH 3 solution used to extract the active;

the dosage form includes 100% Bentonite if saline and EtOH 40% used to extract the active;

the dosage form includes 100% Bentonite if pH 3 solution and EtOH 40% used to extract the active;

the dosage form includes 50% Bentonite and 50% Charcoal if saline and a pH 3 solution used to extract the active;

the dosage form includes 100% Bentonite if water, saline and EtOH 40% used to extract the active;

the dosage form includes 60% Bentonite and 40% Charcoal if water, saline and a pH 3 solution used to extract the active;

the dosage form includes 100% Bentonite if water, a pH 3 solution and EtOH 40% used to extract the active;

the dosage form includes 100% Bentonite if a pH 3 solution, EtOH 40%, and saline used to extract the active;

the dosage form includes 100% Bentonite if water, saline, EtOH 40%, and a pH 3 solution used to extract the active;

the dosage form includes 100% AcDiSol if only water used to extract but 0.1N HCl used to release the active;

the dosage form includes 88% AcDiSol and 12% Charcoal if only EtOH 40% used to extract but 0.1N HCl used to release the active.

the dosage form includes 100% AcDiSol if only a pH 3 solution used to extract but 0.1N HCl used to release the active.

the dosage form includes 60% AcDiSol and 40% Charcoal if water and saline used to extract but 0.1N HCl used to release the active.

the dosage form includes 91% AcDiSol and 9% Charcoal if water and a pH 3 solution used to extract but 0.1N HCl used to release the active;

the dosage form includes 60% AcDiSol and 40% Charcoal if saline and EtOH 40% used to extract but 0.1N HCl used to release the active;

the dosage form includes 85% AcDiSol and 15% Charcoal if EtOH 40% and a pH 3 solution used to extract but 0.1N HCl used to release the active;

the dosage form includes 82% AcDiSol and 18% Charcoal if water, a pH 3 solution and EtOH 40% used to extract but 0.1N HCl used to release the active;

the dosage form includes 60% AcDiSol and 40% Charcoal if water, saline, and a pH 3 solution used to extract but 0.1N HCl used to release the active;

the dosage form includes 60% AcDiSol and 40% Charcoal if water, saline, and EtOH 40% used to extract but 0.1N HCl used to release the active;

the dosage form includes 60% AcDiSol and 40% Charcoal if saline, EtOH 40%, a pH 3 solution used to extract but 0.1N HCl used to release the active;

the dosage form includes 60% AcDiSol and 40% Charcoal if water, saline, EtOH 40%, and a pH 3 solution used to extract but 0.1N HCl used to release the active;

A deterrent composition of Claim 1 wherein all three deterrent agents are coated.

the dosage form wherein only Bentonite is coated.

the dosage form wherein only Charcoal is coated;

the dosage form wherein both Bentonite and Charcoal are coated.

the dosage form wherein all three deterrent agents are non-coated.

the dosage form can be used to trap or to bind charged or non-charged active ingredients including drugs, proteins, toxins, odors, perfumes, and solvents.

In another embodiment of the disclosure, a therapeutic dosage form comprises one or more pharmaceutical active ingredients; a water-swellable superabsorbent polymer, and a plastic agent consisting of a thermoplastic water-soluble or water-insoluble polymer which provides mechanical strength to the structure of the dosage form.

In various embodiments thereof, the superabsorbent polymer absorbs at least 40 g/g of deionized water at room temperature; the superabsorbent polymer is selected from a group consisting of: chemically-crosslinked homopolymers, copolymers or terpolymers of water-soluble monomers of sodium acrylate, potassium acrylate, sodium methacrylate, potassium methacrylate, potassium sulfopropyl acrylate, acrylamide, 2-acrylamido 2-methyl 1-propane sulfonic acid, and methacrylamidopropyltrimethyl ammonium chloride; the superabsorbent polymer comprises 1-99 wt % of the dosage form; the superabsorbent polymer comprises 15-25 wt % of the dosage form; the plastic agent is a polymer with a glass transition temperature between about 40° C. and about 100° C.; the plastic agent is a polymer with a glass transition temperature between about 40° C. and about 55° C.; the plastic agent is at least one of a low glass transition homopolymers of vinyl acetate and a low glass transition copolymer of vinyl acetate; the plastic agent comprises 1-99 wt % of the dosage form; the plastic agent comprises 15-25 wt % of the dosage form.

In further embodiments thereof, the dosage form further includes a superviscosifier selected from the group consisting of: water soluble polymer, polyethylene oxide, methyl cellulose, hydroxypropyl methylcellulose, carboxymethyl cellulose, hydroxyethyl cellulose, hydroxypropyl cellulose, xanthan gum, guar gum, and non-crosslinked forms of the polymers of the previous paragraph; the dosage form further includes a very high molecular weight polyethylene oxide superviscosifier; the dosage form further includes a polyethylene oxide superviscosifier with molecular weight equal or greater than 5,000,000 Da; the superabsorbent polymer is crosslinked poly(sodium acrylate), and the plastic agent is a physical blend of poly(vinyl acetate) and poly(vinyl pyrrolidone); the plastic agent is Kollidone SR® (BASF); the superabsorbent polymer is crosslinked polyacrylamide, and the plastic agent is a physical blend of poly(vinyl acetate) and poly(vinyl pyrrolidone); the superabsorbent polymer is crosslinked poly(sulfopropyl acrylate potassium), and the plastic agent is a physical blend of poly(vinyl acetate) and poly(vinyl pyrrolidone); the superabsorbent polymer is crosslinked poly(2-acrylamido-propane sulfonic acid), and the plastic agent is a physical blend of poly(vinyl acetate) and poly(vinyl pyrrolidone); the dosage form further includes polyethylene oxide as a superviscosifying polymer; the dosage form is formed by heat-treating the dosage form at a temperature above the glass transition temperature of the plastic agent.

In an embodiment of the disclosure, a method of at least one of treating acute alcohol intoxication, treating alcohol abuse, and promoting alcohol cessation, comprises providing a dosage form including a superabsorbent polymer operative to absorb alcohol.

In yet another embodiment of the disclosure, a therapeutic dosage form, comprising one or more superabsorbent polymers operative to absorb significantly more alcohol than the weight of the superabsorbent polymer.

In variations thereof, the superabsorbent polymer swells in deionized water from about 100 g/g to about 1000 g/g; the superabsorbent polymer swells in deionized water from about 300 g/g to about 600 g/g within 15 minute swelling time under mixing at room temperature; the superabsorbent polymer is selected from the group consisting of: chemically-crosslinked homopolymers, copolymers or terpolymers of water-soluble and alcohol-soluble monomers of acrylic acid and its salts, methacrylic acid and its salts, sulfopropyl acrylic acid and its salts, acrylamide, 2-acrylamido 2-methyl 1-propane sulfonic acid, and methacrylamidopropyltrimethyl ammonium chloride; the superabsorbent polymer is at least one of an acrylamide based homopolymer, acrylamide based copolymer, or acrylamide based terpolymer; the superabsorbent polymer is chemically crosslinked polyacrylamide; the superabsorbent polymer comprises 1 to 100 wt % of the composition.

In other embodiments thereof, the dosage form further comprises a superviscosifier selected from the group consisting of water soluble polymers with high affinity for alcohol: polyethylene oxide, methyl cellulose, hydroxypropyl methylcellulose, carboxymethyl cellulose, hydroxyethyl cellulose, hydroxypropyl cellulose, xanthan gum, guar gum, and the non-crosslinked polymers of the preceding paragraph; and/or the superviscosifier is very high molecular weight polyethylene oxide.

In other embodiments thereof, the Cone & Plate shear viscosity of the 2 w/v % solution of the superviscosifier in 20 v/v % ethanol in water at 22-24° C. and shear rate of 2 sec⁻¹ is from about 5200 to about 12000 cP; the Cone & Plate shear viscosity of the 2 w/v % solution of the superviscosifier in 20 v/v % ethanol in water at 22-24° C. and shear rate of 2 sec⁻¹ is advantageously from about 7800 to about 9600 cP; the Cone & Plate shear viscosity of the 2 wt % solution of the superviscosifier in 60 v/v % ethanol in water at 22-24° C. and shear rate of 20 sec⁻¹ is from about 1200 to about 3000 cP; and/or the Cone & Plate shear viscosity of the 2 wt % solution of the superviscosifier in 60 v/v % ethanol in water at 22-24° C. and shear rate of 20 sec⁻¹ is advantageously from about 1900 to about 2300 cP.

In additional embodiments thereof, the superviscosifier is polyethylene oxide at molecular weights equal or greater than 5,000,000 Da; the Cone & Plate shear viscosity of a 2 w/v % solution of the superviscosifier in water at 22-24° C. and a shear rate of 2 sec⁻¹ is from about 4700 to about 11,100 cP; the viscosity at shear rate of 2 sec⁻¹ is from about 7100 to about 8700 cP; the dosage form further includes 1-99 wt % of the superviscosifier; the dosage form comprising 50-99% of superabsorbent and 1-50% of the superviscosifier, when the hydroalcoholic solution contains less than 40% ethanol; the dosage form includes 1-50% of superabsorbent and 50-99% of the superviscosifier, when the hydroalcoholic solution contains greater than 40% of ethanol; the superabsorbent polymer is crosslinked polyacrylamide and the superviscosifier is polyethylene oxide; and/or the superabsorbent polymer is crosslinked poly (2-acrylamido-propane sulfonic acid), and the superviscosifier is polyethylene oxide.

In yet further embodiments thereof; the dosage form is formed as one of a tablet, capsule, gel, or patch; the dosage form further includes a pharmaceutically active ingredient; the dosage form further including at least one of tablet excipients for tableting, capsule excipients for encapsulation, and patch excipients for transdermal patches; the one or more pharmaceutically active ingredients treats an illness selected from the group consisting: anxiety, depression, sleep disorders, pain, lack of energy, attention deficit, cough, and cold; the one or more pharmaceutically active ingredients is selected from the group of barbiturates comprising: phenobarbitals, benzodiazepines, codeine, morphine, oxycodone, oxymorphone, hydrocodone, hydromorphone, Tramadol, amphetamines, methyl phenidate, dextromethorphan, and pseudoephedrine; the one or more pharmaceutically active ingredients is in the form of its weak base; the dosage form is a tablet; the dosage form is a capsule; the dosage has a form selected from the group consisting of gel, suppository, suspension, emulsion, micro sized dispersion, nano-sized dispersion, semi-solid, paste, ointment, lozenge, strip, film, and rod.

In further embodiments thereof, the superabsorbent polymer can freely swell in 5 wt % aqueous ethanol from about 100 g/g to about 1000 g/g, most practically from about 280 g/g to about 500 g/g in at least 15 minute swelling time under mixing;

the superabsorbent polymer can freely swell in 10 wt % aqueous ethanol from about 100 g/g to about 1000 g/g, most practically from 260 g/g to about 480 g/g in at least 15 minute swelling time under mixing;

the superabsorbent polymer can freely swell in 40 wt % aqueous ethanol from about 100 g/g to about 1000 g/g, most practically from 200 g/g to about 375 g/g in at least 15 minute swelling time under mixing;

the superabsorbent polymer can freely swell in an acidic aqueous solution (pH 3) from about 100 g/g to about 1000 g/g, most practically from 190 g/g to about 360 g/g in at least 15 minute swelling time under mixing;

the superabsorbent polymer can freely swell in an acidic aqueous solution (pH 4) from about 100 g/g to about 1000 g/g, most practically from 280 g/g to about 520 g/g in at least 15 minute swelling time under mixing;

the superabsorbent polymer can freely swell in an acidic aqueous solution (pH 5) from about 100 g/g to about 1000 g/g, most practically from 290 g/g to about 550 g/g in at least 15 minute swelling time under mixing;

the Cone & Plate shear viscosity of the 2 w/v % solution of the superviscosifier in 20 v/v % ethanol in water at 22-24° C. and shear rate of 2 sec⁻¹ is from 5200-12000 cP, advantageously from 7800-9600 cP;

the Cone & Plate shear viscosity of the 2 w/v % solution of the superviscosifier in 40 v/v % ethanol in water at 22-24° C. and shear rate of 2 sec⁻¹ is from 5700-13300 cP, advantageously from 8500-10400 cP;

the Cone & Plate shear viscosity of the 2 w/v % solution of the superviscosifier in 60 v/v % ethanol in water at 22-24° C. and shear rate of 2 sec⁻¹ is from 6100-14400 cP, advantageously from 9200-11300 cP;

the Cone & Plate shear viscosity of the 2 w/v % solution of the superviscosifier in 80 v/v % ethanol in water at 22-24° C. and shear rate of 2 sec⁻¹ is from 6100-14400 cP, advantageously from 9200-11300 cP;

the Cone & Plate shear viscosity of the 2 w/v % solution of the superviscosifier in water at 22-24° C. and shear rate of 20 sec⁻¹ is from 1000-2400 cP, advantageously from 1500-1900 cP;

the Cone & Plate shear viscosity of the 2 w/v % solution of the superviscosifier in 20 v/v % ethanol in water at 22-24° C. and shear rate of 20 sec⁻¹ is from 1000-2500 cP, advantageously from 1600-2000 cP;

the Cone & Plate shear viscosity of the 2 w/v % solution of the superviscosifier in 40 v/v % ethanol in water at 22-24° C. and shear rate of 20 sec⁻¹ is from 1200-2800 cP, advantageously from 1800-2200 cP;

the Cone & Plate shear viscosity of the 2 wt % solution of the superviscosifier in 60 v/v % ethanol in water at 22-24° C. and shear rate of 20 sec⁻¹ is from 1200-3000 cP, advantageously from 1900-2300 cP;

the Cone & Plate shear viscosity of the 2 wt % solution of the superviscosifier in 80 v/v % ethanol in water at 22-24° C. and shear rate of 20 sec⁻¹ is from 1200-3000 cP, advantageously from 1900-2300 cP;

the Cone & Plate shear viscosity of the 2 wt % solution of the superviscosifier in water at 22-24° C. and shear rate of 40 sec⁻¹ is from 600-1600 cP, advantageously from 1000-1200 cP;

the Cone & Plate shear viscosity of the 2 wt % solution of the superviscosifier in 20 v/v % ethanol in water at 22-24° C. and shear rate of 40 sec⁻¹ is from 700-1700 cP, advantageously from 1100-1300 cP;

the Cone & Plate shear viscosity of the 2 wt % solution of the superviscosifier in 40 v/v % ethanol in water at 22-24° C. and shear rate of 40 sec⁻¹ is from 800-2000 cP, advantageously from 1200-1500 cP;

the Cone & Plate shear viscosity of the 2 wt % solution of the superviscosifier in 60 v/v % ethanol in water at 22-24° C. and shear rate of 40 sec⁻¹ is from 900-2100 cP, advantageously from 1300-1600 cP; and/or

the Cone & Plate shear viscosity of the 2 wt % solution of the superviscosifier in 80 v/v % ethanol in water at 22-24° C. and shear rate of 40 sec⁻¹ is from 800-2000 cP, advantageously from 1300-1600 cP.

In another embodiment of the disclosure, a therapeutic dosage form comprises at least one pharmaceutical active ingredient known to be abusable; a swellable superabsorbent polymer, that once mixed with the drug and other regular tablet excipients and compressed to a tablet, has no retarding or inhibiting effect on drug release in 0.1N HCl when drug release study is conducted according to the USP II method; and a plastic agent having a glass transition temperature ranging 40-100° C. (advantageously ranging 40-55° C.), or having melting temperature ranging 40-100° C. (advantageously ranging 60-75° C.).

In various embodiments thereof, the dosage form further comprises excipients to make a corresponding dosage form, wherein the excipients include tablet excipients for tableting, capsule excipients for encapsulation, or patch excipients for transdermal patches; the pharmaceutical active ingredient treats anxiety, depression, sleep disorders, pain, lack of energy, attention deficit, cough and cold; and/or the pharmaceutical active ingredient is selected from a group of barbiturates such as phenobarbitals, benzodiazepines, codeine, morphine, oxycodone, oxymorphone, hydrocodone, hydromorphone, Tramadol, amphetamines, methyl phenidate, dextromethorphan, and pseudoephedrine.

In other embodiments thereof, the superabsorbent polymer is selected from a group of chemically-crosslinked polymers, copolymers and terpolymers of water-soluble monomers of sodium acrylate, potassium acrylate, sodium methacrylate, potassium methacrylate, potassium sulfopropyl acrylate, acrylamide, 2-acrylamido 2-methyl 1-propane sulfonic acid, and methacrylamidopropyltrimethyl ammonium chloride; the superabsorbent polymer comprises about 1 to about 99 wt % of the composition, advantageously about 20 to about 30 wt % of the composition.

In yet further embodiments thereof, the dosage form further includes a superviscosifier selected from polyacrylic acid crosslinked with allyl ether of pentaerythritol or allyl ether of sucrose; polyethylene oxide, methyl cellulose, hydroxypropyl methylcellulose, carboxymethyl cellulose, hydroxyethyl cellulose, hydroxypropyl cellulose, guar gum, and non-crosslinked polymers of the preceding paragraph; and/or the superviscosifier is a very high molecular weight polyethylene oxide, such as Polyox WSR® Coagulant (BASF).

In various further embodiments:

a 300 mg composition containing 25 wt % of crosslinked poly(sodium acrylate), after being placed in 10 mL of deionized water for 2 minutes, provides 0 mL of filtrate (amount passing through the filter);

a 300 mg composition containing 25 wt % of crosslinked poly(sodium acrylate), after being placed in 10 mL of EtOH 5 v/v % (aq) for 2 minutes, provides 0-1 mL of filtrate (amount passing through the filter);

a 300 mg composition containing 25 wt % of crosslinked poly(sodium acrylate), after being placed in 10 mL of EtOH 10 v/v % (aq) for 2 minutes, provides 2-3 mL of filtrate (amount passing through the filter);

a 300 mg composition containing 25 wt % of crosslinked poly(sodium acrylate), after being placed in 10 mL of EtOH 20 v/v % (aq) for 2 minutes, provides 5-6 mL of filtrate (amount passing through the filter);

a 300 mg composition containing 25 wt % of crosslinked poly(sodium acrylate) and 5 wt % very high molecular weight polyethylene oxide (about 5,000,000 Da), after being placed in 10 mL of EtOH 5 v/v % (aq) for 2 minutes, provides 0-1 mL of filtrate (amount passing through the filter);

a 300 mg composition containing 25 wt % of crosslinked poly(sodium acrylate) and 5 wt % very high molecular weight polyethylene oxide (about 5,000,000 Da), after being placed in 10 mL of EtOH 10 v/v % (aq) for 2 minutes, provides 0-1 mL of filtrate (amount passing through the filter);

a 300 mg composition containing 25 wt % of crosslinked poly(sodium acrylate) and 5 wt % very high molecular weight polyethylene oxide (about 5,000,000 Da), after being placed in 10 mL of EtOH 20 v/v % (aq) for 2 minutes, provides 3-4 mL of filtrate (amount passing through the filter);

a 300 mg composition containing 25 wt % of crosslinked polyacrylamide, after being placed in 10 mL of water-ethanol mixtures containing 0-20 v/v % ethanol, provides 0 mL of filtrate (amount passing through the filter);

a 300 mg composition containing 25 wt % of crosslinked polyacrylamide, after being placed in 10 mL of EtOH 30 v/v % (aq) for 2 minutes, provides 0-1 mL of filtrate (amount passing through the filter);

a 300 mg composition containing 25 wt % of crosslinked polyacrylamide, after being placed in 10 mL of EtOH 40 v/v % (aq) for 2 minutes, provides 0.5-2 mL of filtrate (amount passing through the filter);

a 300 mg composition containing 25 wt % of crosslinked polyacrylamide, after being placed in 10 mL of EtOH 50 v/v % (aq) for 2 minutes, provides 3-4 mL of filtrate (amount passing through the filter);

a 300 mg composition containing 25 wt % of crosslinked polyacrylamide and 5 wt % of very high molecular weight polyethylene oxide (5,000,000 Da), after being placed in 10 mL of EtOH 40 v/v % (aq) for 2 minutes, provides 0-1 mL of filtrate (amount passing through the filter);

a 300 mg composition containing 25 wt % of crosslinked poly(sulfopropylacrylate potassium), after being placed in 10 mL of an water-ethanol mixture containing 0-60 v/v % ethyl alcohol, provides same amount of filtrate (amount passing through the filter).

In still further embodiments, the plastic agent is selected from a family of vinyl acetate homopolymers or its copolymers containing over 50% vinyl acetate monomer; the plastic agent of about 1 to about 99 wt % of the composition, advantageously about 15 to about 25 wt % of the composition; the superabsorbent polymer is crosslinked poly(sodium acrylate), and the plastic agent is a physical blend of poly(vinyl acetate) and poly(vinyl pyrrolidone) (such as Kollidone SR® (BASF); the superabsorbent polymer is crosslinked polyacrylamide, and the plastic agent is a physical blend of poly(vinyl acetate) and poly(vinyl pyrrolidone) (such as Kollidone SR® (BASF); the superabsorbent polymer is crosslinked poly(sulfopropylacrylate potassium), and the plastic agent is a physical blend of poly(vinyl acetate) and poly(vinyl pyrrolidone) (such as Kollidone SR® (BASF); the superabsorbent polymer is crosslinked poly(2-acrylamido-propane sulfonic acid), and the plastic agent is a physical blend of poly(vinyl acetate) and poly(vinyl pyrrolidone) (such as Kollidone SR® (BASF), and/or the dosage form includes polyethylene oxide; the composition is further heat-treated at above the glass transition temperature of the hydrophobic plastic agent or at above the melting point of the hydrophilic plastic agent; the composition is a single layer matrix tablet; the composition is a bi- or multiple layer tablet; the dosage form is encapsulated in an orally administrable capsule such as in gelatin or hydroxypropyl methylcellulose capsules.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 shows a tablet according to one embodiment of the disclosure.

FIG. 2 shows absorption for a tablet according to one embodiment of the disclosure.

FIG. 3 shows ultimate swelling and deterrence capacity in hydroalcoholic solutions for tablets according to embodiments of the disclosure.

FIG. 4 shows ultimate swelling and deterrence capacity in hydroalcoholic solutions for a tablet according to and embodiment of the disclosure.

FIG. 5 shows the relationship between the degree of crosslinking and the swelling capacity.

FIG. 6 illustrates an effect of the superabsorbent polymer on extracting solution (whole tablet).

FIG. 7 illustrates an effect of the superabsorbent polymer on extracting solution (crushed tablet.

FIG. 8 illustrates an effect of the use of plastic agent and the heat treatment on tablet crushability.

FIGS. 9A to 9E show linear and crosslinked polyacids that can be used in embodiments of the disclosure.

FIG. 10 shows the deterrent effect of IC-SCMC.

FIG. 11 shows the binding effect of IC-SCMC with respect to pH.

FIG. 12 illustrates that heating does not pose any negative effect on binding capacity of IC-SCMC.

FIG. 13 illustrates that hydroalcoholic solutions containing up to 40 wt % EtOH do not affect the binding capacity of IC-SCMC with Tramadol.

FIG. 14 illustrates the relationship between drug release and time for different tablets according to the disclosure.

FIG. 15 illustrates the deterrent effect of IC-SCMC

FIG. 16 illustrates the binding effect of IC-SCMC with respect to pH.

FIG. 17 illustrates the relationship between drug release and time for different tablets according to the disclosure.

FIG. 18 illustrates that physically-crosslinked carboxymethyl cellulose does not display deterrence potential.

FIG. 19 shows that IC-PVP does not display deterrent capacity for Tramadol HCl.

FIG. 20 shows that tablets containing different amounts of IC-PVP are not abuse-deterrent.

FIG. 21 shows the effectiveness of different deterrents.

FIG. 22 shows release of Tramadol in 0.1N HCl solution.

FIGS. 23 and 24 schematically show entrapment of alcohol molecules.

FIG. 25 illustrates volumetric swelling of crosslinked poly(sodium acrylate) in different alcoholic solutions.

FIG. 26 illustrates volumetric swelling of crosslinked polyacrylamide in different alcoholic solutions.

FIG. 27 illustrates volumetric swelling of crosslinked copolymer of sodium acrylate and acrylamide in different alcoholic solutions.

FIG. 28 illustrates volumetric swelling of crosslinked poly(potassium salt of sulfopropyl acrylate) with superporous structure in different alcoholic solutions.

FIG. 29 illustrates volume swelling capacity of crosslinked poly(sodium acrylate), crosslinked polyacrylamide, and crosslinked sodium acrylate and acrylamide copolymer in hydroalcoholic solutions containing 0-50% ethyl alcohol.

FIG. 30 illustrates swelling capacity of crosslinked polyacrylamide in 5 wt % EtOH solution.

FIG. 31 illustrates swelling capacity of crosslinked polyacrylamide in 10 wt % EtOH solution.

FIG. 32 illustrates swelling capacity of crosslinked polyacrylamide in 20 wt % EtOH solution.

FIG. 33 illustrates swelling capacity of crosslinked polyacrylamide in 40 wt % EtOH solution.

FIG. 34 illustrates weight swelling capacity of crosslinked polyacrylamide in different hydroalcoholic solutions at pH of 7.

FIG. 35 illustrates weight swelling capacity of crosslinked polyacrylamide in different pH medium without and with ethanol.

FIG. 36 illustrates weight swelling capacity of crosslinked polyacrylamide in acidic solutions versus in acidic solutions containing 5% ethanol.

FIG. 37 illustrates weight swelling capacity of crosslinked polyacrylamide in different hydro-alcoholic solutions measured by bag versus sieve methods.

FIG. 38 illustrates cone & plate shear viscosity of 2 wt % solution of Polyox WSR in different alcoholic solutions measured at shear rate of 2 sec⁻¹ and temperature of 22-24° C.

FIG. 39 illustrates cone & plate shear viscosity of 2 wt % solution of Polyox WSR in different alcoholic solutions measured at shear rate of 20 sec⁻¹ and temperature of 22-24° C.

FIG. 40 illustrates cone & plate shear viscosity of 2 wt % solution of Polyox WSR in different alcoholic solutions measured at shear rate of 40 sec⁻¹ and temperature of 22-24° C.

FIG. 41 illustrates that Tramadol HCl can effectively be captured by bentonite clay.

FIG. 42 illustrates that HPMC can effectively reduce the binding effect of the clay granulated particles.

FIGS. 43A and 43B illustrate that clay is more effective at higher concentration in the tablet.

FIG. 44 illustrates the effect of enteric coating on binding capacity of the clay particles.

FIG. 45 illustrates the stability of the clay-drug complex at different pHs, especially at low pHs.

FIG. 46 illustrates stability of drug clay complex in different hydroalcoholic solutions.

FIG. 47 illustrates the amount of Tramadol released from the drug-clay complex in different extraction or dissolution medium.

FIG. 48 illustrates particles, aggregates and dosage of activated charcoal.

FIG. 49 illustrates effective adsorption of Tramadol into charcoal particles.

FIG. 50 illustrates the effect of coating on Tramadol adsorption into charcoal aggregates.

FIGS. 51 and 52 illustrate release and adsorption profiles of the tablet formulations containing different Tramadol charcoal compositions.

FIG. 53 illustrates the effect of pH on charcoal Tramadol adsorption.

FIG. 54 illustrates the effect of alcohol on charcoal adsorption of Tramadol HCl.

FIG. 55 illustrates Tramadol release from SAP tablets containing low and high concentrations of either polyacrylamide or poly(sodium acrylate).

FIG. 56 illustrates the amount of extraction volume recovery for control tablet and tablets containing polyacrylamide, poly(sodium acrylate) or their copolymer.

FIG. 57 shows a calibration curve in water.

FIG. 58 shows a calibration curve in 0.1 N HCl.

FIG. 59 shows a calibration curve in 0.9% normal saline.

FIG. 60 shows a calibration curve in EtOH 40%.

FIG. 61 shows a calibration curve in pH3 solution.

FIG. 62 shows extraction study in water results after 10 minutes.

FIG. 63 shows extraction study in 0.1 N HCl results after 10 minutes.

FIG. 64 shows extraction study in 0.9% normal saline results after 10 minutes.

FIG. 65 shows extraction study in EtOH results after 10 minutes.

FIG. 66 shows extraction study in pH3 solution after 10 minutes.

FIG. 67 shows drug trapped percent for different medium.

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

The disclosure describes the use of certain pharmaceutically acceptable functional polymers that are used to make more effective abuse deterrent medications. This disclosure describes different approaches that can potentially deter abuse by reducing the efficacy of main processes utilized by abusers to speed drug absorption and enhance its effect. Pharmaceutical compositions of the disclosure incorporate one or more of the following elements described herein to reduce abuse: super water-absorbency, alcohol absorption, organic binding agents, inorganic binding agents, adsorption, and tough platforms. These compositions of the disclosure are safe and effective if used by regular patients or as prescribed, and are also ineffective or less effective in the hand of abusers. Herein, drug refers to a pharmaceutically active ingredient, which is incorporated into a dosage form of the disclosure.

In an embodiment, a pharmaceutical composition of this disclosure is composed of an abusable drug active ingredient, and two primary polymers. The primary polymers utilized in this disclosure are an integral part of the abusable formulation. The first primary polymer, a water-swellable superabsorbent polymer, is a chemically-crosslinked hydrophilic polymer or copolymer, which can at least swell in water to greater than 40 grams per gram of the dry polymer. The water-swellable superabsorbent polymers of this disclosure will change the texture and the flow property of the dosage form in the solution state. Depending on its concentration in the tablet, this polymer significantly reduces the amount of filtrate during the extraction process. The second primary polymer, a plastic agent, is a thermoplastic water-soluble or water-insoluble polymer, which provides mechanical property to the dosage form in the solid state.

Abusers generally utilize crushing and extraction processes in order to retrieve the high concentration of the active ingredient from the original dosage form. Once crushed, they will either directly abuse it by insufflation, or they add the crushed powder into an aqueous solution or a hydro-alcoholic solution for further extraction of the active ingredient(s).

In one form of abuse, the abuser will use the whole tablet with an ingestion of alcohol. The primary polymers of this disclosure increase the resistance of the tablet to mechanical crushing, and change the solution state of the extraction medium into a solid gel, by which no or minimum drug will be extracted from the abuse-deterred dosage form.

The primary polymers of this disclosure can operate to produce no change, or an insignificant change in the release profile of the active ingredient in the acidic environment of the stomach, when used as intended for a regular patient. Polymers of this disclosure can be physically mixed with the active ingredient to make a matrix tablet, or can be used as a separate layer to make bi- or multiple layer tablets, or can be used in the preparation of other dosage forms.

The disclosure enables the formation of prescription drugs less likely to be abused by the most common methods of medication tampering. The disclosure addresses each tampering method, and defines a way to lessen its likelihood of occurring. This disclosure thus targets multiple methods of abuse with the use of one or more polymers that can be incorporated into the current methods of tablet manufacturing.

The following points highlight the theoretical concept and approach for discouraging or preventing each type of tampering method.

CRUSHING: Prospective abusers crush tablets containing potent pharmaceutical ingredients that can directly be snorted into the nose. The active medication is quickly absorbed through the nasal tissue and into the blood stream giving the abuser a quick “high” and a euphoric or desired feeling.

According to an embodiment of the disclosure, primary superabsorbent polymers will be added to tablets, and upon being crushed and inhaled, will swell and form a gel layer when in contact with the wet nasal lining. The changing of dry powder into a gel mass in the nose also “traps” the drug and prevents its quick release into the blood. These two effects are intended to discourage abuse by the nasal route and slow release of the drug into the bloodstream. Moreover the primary plastic agent incorporated into the tablet formulation causes the tablet to be crushed into much larger pieces, and makes the overall crushing process more difficult. As opposed to fine particles, large pieces of crushed tablet with less contact surface area provide a slower drug release into the nasal lining in case of insufflation, and/or act to retard the dissolution and extraction in case of abuse by injection.

INTRAVENOUS (IV) ABUSE: After successfully crushing a tablet containing a drug for abuse, the powder is dissolved in water, alcohol, or other available liquids. The mixture is then filtered to remove any un-dissolved material before being drawn up into a syringe and injected. This results in a large amount of drug entering the body at once and provides the user with a powerful “rush” and euphoric effect.

In accordance with the disclosure, water-swellable superabsorbent polymers can be incorporated into the tablet to deter this type of abuse. After a tablet containing one or more of these polymers is crushed and mixed with an appropriate amount of liquid needed for intravenous injection, the powder in the liquid medium, in a very short period of time turns into a swollen gel that traps the active drug and liquid. The water-swollen mass cannot be filtered using a regular filter paper such as coffee filter paper, or lab filter papers. This approach is therefore designed to impede the ability to abuse a tablet by intravenous injection.

ALCOHOL CO-INGESTION: Swallowing the tablet medication (whole tablet or crushed) with alcohol is commonly experienced to enhance the effect of both drug and alcohol. For those drugs that dissolve in alcohol, this act also gives the user a quicker euphoric feeling since the drug can dissolve and enter the bloodstream more quickly.

In accordance with the disclosure, alcohophilic superabsorbent polymers can be added to the tablet, which when swallowed with alcohol, absorb and trap both alcohol and the dissolved drug so its quick absorption and euphoric effects are less likely to occur.

The inventors have determined that advantageous polymer properties for abuse deterrent applications include characteristics for 1) interacting with moisture in the air when exposed from a crushed tablet, 2) swelling and gelling in water and hydro-alcoholic solutions which are used by abusers to tamper with the medication, and 3) absorbing alcohol and soluble drug when medication is co-ingested with alcoholic beverages.

Polymers with great affinity for water tend to display the least affinity for alcohol, and vice versa. Alternatively stated, a polymer that absorbs significant amounts of water or significantly increases the viscosity of an aqueous solution, will experience a very weak interaction with water if alcohol is added into an aqueous solution. The disclosure identifies specific types of polymers with moderate affinity for both water and alcohol, and/or polymer combinations where one has good affinity for water and the other a good affinity for alcohol.

In accordance with the disclosure, primary superabsorbent polymers advantageously can be: made of very hydrophilic monomers, ionics and non-ionics; chemically crosslinked; absorbent of an aqueous medium rich in water; absorbent of an aqueous medium rich in alcohol; and very hygroscopic. In addition, they can: form an integral part of the formulation; prevent crushed medication particles from becoming free flowing under any abusable action such as snorting; effectively prevent filterability and impede the ability to abuse a tablet by intravenous injection; trap the drug dissolved in the hydroalcoholic solution and prevent its rapid absorption and euphoric effects when swallowed with alcoholic beverages.

Examples of such polymers include crosslinked polymers, copolymers and terpolymers of water-soluble monomers of sodium acrylate, potassium acrylate, sodium methacrylate, potassium methacrylate, potassium sulfopropyl acrylate, acrylamide, 2-acrylamido 2-methyl 1-propane sulfonic acid (AMPS), and methacrylamidopropyltrimethyl ammonium chloride.

Superabsorbent polymers of this disclosure include crosslinked poly(sodium acrylate), crosslinked poly(sulfopropyl acrylate potassium), crosslinked polyacrylamide, crosslinked copolymer of acrylamide and sodium acrylate. Synthetic polymers of this disclosure can be prepared following a general experimental procedure that we previously reported [26-29] which are incorporated herein by reference, or their purified commercial counterparts can be used instead.

An additional component includes a primary plastic agent, which advantageously: is soluble or insoluble in water; has good thermoplastic properties; and has binding and adhesion properties. Additionally, the plastic agent should be capable of being processed at relatively low temperature in order to avoid drug thermal decomposition. The inventors have found these materials generally have glass transition temperature at around 35-55° C.

Plastic agents used in this disclosure can be blends of polyvinyl acetate and other polymers, or copolymers of vinyl acetate and other monomers.

While the foregoing primary polymers can provide sufficient performance to deter abuse, secondary polymers, which can serve as superviscosifier polymers, can be advantageously used along with the primary polymers to enhance the deterrence capacity of the dosage form. A superviscosifier is a very high molecular weight polymer with great affinity for both water and alcohol. In other words, a superviscosifier can provide significant viscosity in both aqueous and hydroalcoholic (very rich in alcohol) solutions.

Secondary polymers (Superviscosifier polymers) are advantageously made of very hydrophilic monomers, ionic and non-ionics; are not chemically crosslinked; enhance viscosity of the aqueous medium rich in water; and enhance viscosity of the aqueous medium rich in alcohol. Their function can be only to enhance the efficacy of the primary polymers used in the formulation. The secondary polymers contribute to preventing filterability and impeding the ability to abuse a tablet by intravenous injection.

Examples of such polymers include polyethylene oxide, methyl cellulose, hydroxypropyl methylcellulose, carboxymethyl cellulose, hydroxyethyl cellulose, hydroxypropyl cellulose, guar gum, and xanthan.

In the examples, TRAMADOL is used is representative of a pharmaceutically active ingredient. It should be understood that other drugs can be used, as described elsewhere herein.

### EXPERIMENTAL PROCEDURES & MEASUREMENTS

Filterability: A composition or a tablet containing an active, primary and secondary polymers (if used), and Prosolv (silicified microcrystalline cellulose) was crushed in a pestle and mortar, and mixed with 10 mL of liquid medium including deionized water, hydro-alcoholic solutions at different alcohol concentration, pure ethanol, and saline. After 2 minutes, the dispersion was filtered and the amount of filtrate (passed through the filter) was measured by volume and weight.

Drug Extraction: The extract from step 1 (if any) was examined with a UV-Vis to determine the amount of the active ingredient extracted.

Drug Release: Same composition as in step 1 was placed into a dissolution medium (water or 0.1N HCl), and was tested for the drug release according to the USP standard.

### Examples

### Single-Layer Matrix Tablets

Filterability: Different compositions were prepared and tested in different extracting medium, and the amount of filtrate was measured. Since only the liquid part (filtrate) of the extraction medium can be drawn up into syringe, this test will show how effective the superabsorbent polymers are to decrease the amount of filtrates in different hydro-alcoholic solutions.

Compositions Containing Superabsorbent Polymer Only

Filterability of compositions (300 mg) containing Prosolv, and crosslinked poly(sodium acrylate) at different superabsorbent concentration, after 2 minutes in deionized water:

| | | | | Prosolv | | | Fil- |
|---|---|---|---|---|---|---|---|
| | | Polymer | Polymer | SMCC | | Filtrate | trate |
| | Polymer | % | (mg) | 90 (mg) | Solution | (ml) | (g) |
| 1 | poly(sodium | 0% | 0 | 300 | H₂O | 9.1 | 9.21 |
| | acrylate) | | | | | | |
| 2 | poly(sodium | 0% | 0 | 300 | H₂O | 9.1 | 9.21 |
| | acrylate) | | | | | | |
| 3 | poly(sodium | 0% | 0 | 300 | H₂O | 9.1 | 9.15 |
| | acrylate) | | | | | | |
| 4 | poly(sodium | 5% | 15 | 285 | H₂O | 6.5 | 6.61 |
| | acrylate) | | | | | | |
| 5 | poly(sodium | 5% | 15 | 285 | H₂O | 6.4 | 6.47 |
| | acrylate) | | | | | | |
| 6 | poly(sodium | 5% | 15 | 285 | H₂O | 6.4 | 6.52 |
| | acrylate) | | | | | | |
| 7 | poly(sodium | 15% | 45 | 255 | H₂O | 2.6 | 2.66 |
| | acrylate) | | | | | | |
| 8 | poly(sodium | 15% | 45 | 255 | H₂O | 3.5 | 3.50 |
| | acrylate) | | | | | | |
| 9 | poly(sodium | 15% | 45 | 255 | H₂O | 2.8 | 2.87 |
| | acrylate) | | | | | | |
| 10 | poly(sodium | 20% | 60 | 240 | H₂O | <1 ml | 0.21 |
| | acrylate) | | | | | | |
| 11 | poly(sodium | 20% | 60 | 240 | H₂O | <1 ml | 0.69 |
| | acrylate) | | | | | | |
| 12 | poly(sodium | 20% | 60 | 240 | H₂O | <1 ml | 0.83 |
| | acrylate) | | | | | | |

Filterability of compositions (300 mg) containing Prosolv and poly(sodium acrylate) in different solutions:

| | | | | Prosolv | | | |
|---|---|---|---|---|---|---|---|
| | | | Polymer | SMCC 90 | | Filtrate | Filtrate |
| | Polymer | Polymer % | (mg) | (mg) | Solution | (ml) | (g) |
| 13 | poly(sodium | 25% | 75 | 225 | 0% ETOH | <1 ml | 0.17 |
| | acrylate) | | | | | | |
| 14 | poly(sodium | 25% | 75 | 225 | 0% ETOH | 0 | 0.00 |
| | acrylate) | | | | | | |
| 15 | poly(sodium | 25% | 75 | 225 | 0% ETOH | 0 | 0.00 |
| | acrylate) | | | | | | |
| 16 | poly(sodium | 25% | 75 | 225 | 5% ETOH | 0 | 0.00 |
| | acrylate) | | | | | | |
| 17 | poly(sodium | 25% | 75 | 225 | 5% ETOH | <1 | 0.68 |
| | acrylate) | | | | | | |
| 18 | poly(sodium | 25% | 75 | 225 | 5% ETOH | <1 | 0.95 |
| | acrylate) | | | | | | |
| 19 | poly(sodium | 25% | 75 | 225 | 10% | 2.7 | 2.69 |
| | acrylate) | | | | ETOH | | |
| 20 | poly(sodium | 25% | 75 | 225 | 10% | 2.1 | 2.17 |
| | acrylate) | | | | ETOH | | |
| 21 | poly(sodium | 25% | 75 | 225 | 10% | 2.5 | 2.58 |
| | acrylate) | | | | ETOH | | |
| 22 | poly(sodium | 25% | 75 | 225 | 20% | 5 | 4.92 |
| | acrylate) | | | | ETOH | | |
| 23 | poly(sodium | 25% | 75 | 225 | 20% | 5 | 4.92 |
| | acrylate) | | | | ETOH | | |
| 24 | poly(sodium | 25% | 75 | 225 | 20% | 5.1 | 5.04 |
| | acrylate) | | | | ETOH | | |
| 25 | poly(sodium | 25% | 75 | 225 | 40% | 8.3 | 7.88 |
| | acrylate) | | | | ETOH | | |
| 26 | poly(sodium | 25% | 75 | 225 | 60% | 8.3 | 7.68 |
| | acrylate) | | | | ETOH | | |
| 27 | poly(sodium | 25% | 75 | 225 | 80% | 8.5 | 7.32 |
| | acrylate) | | | | ETOH | | |
| 28 | poly(sodium | 25% | 75 | 225 | 100% | 9.2 | 7.34 |
| | acrylate) | | | | ETOH | | |
| 29 | poly(sodium | 25% | 75 | 225 | 100% | 9.1 | 7.21 |
| | acrylate) | | | | ETOH | | |
| 30 | poly(sodium | 25% | 75 | 225 | 100% | 8.9 | 7.05 |
| | acrylate) | | | | ETOH | | |
| 31 | poly(sodium | 25% | 75 | 225 | 0.9% NaCl | 7.2 | 7.35 |
| | acrylate) | | | | | | |
| 32 | poly(sodium | 25% | 75 | 225 | 0.9% NaCl | 7 | 7.10 |
| | acrylate) | | | | | | |
| 33 | poly(sodium | 25% | 75 | 225 | 0.9% NaCl | 7.1 | 7.17 |
| | acrylate) | | | | | | |

Filterability of compositions (300 mg) containing Prosolv and poly(sodium acrylate) and polyethylene oxide in different solutions:

| | | | | Prosolv | | | |
|---|---|---|---|---|---|---|---|
| | | | Polymer | SMCC 90 | | Filtrate | Filtrate |
| | Polymer | Polymer % | (mg) | (mg) | Solution | (ml) | (g) |
| 34 | poly(sodium | 25% | 75 | 210 | 5% | 0.69 | 0.69 |
| | acrylate) + PEO(5%) | | | | ETOH | | |
| 35 | poly(sodium | 25% | 75 | 210 | 10% | 0.7 | 0.79 |
| | acrylate) + PEO(5%) | | | | ETOH | | |
| 36 | poly(sodium | 25% | 75 | 210 | 20% | 3.5 | 3.50 |
| | acrylate) + PEO(5%) | | | | ETOH | | |
| 37 | poly(sodium | 25% | 75 | 210 | 40% | 8.3 | 7.91 |
| | acrylate) + PEO(5%) | | | | ETOH | | |
| 38 | poly(sodium | 25% | 75 | 210 | 80% | 8.1 | 7.03 |
| | acrylate) + PEO(5%) | | | | ETOH | | |
| 39 | poly(sodium | 25% | 75 | 210 | 100% | 9.3 | 7.43 |
| | acrylate) + PEO(5%) | | | | ETOH | | |
| 40 | poly(sodium | 25% | 75 | 210 | 0.9% | 6.7 | 6.78 |
| | acrylate) + PEO(5%) | | | | NaCl | | |

Filterability of compositions (300 mg) containing Prosolv and crosslinked poly(sulfopropyl acrylate potassium) in different solutions; last two compositions contain polyethylene oxide:

| | | | | Prosolv | | | |
|---|---|---|---|---|---|---|---|
| | | | Polymer | SMCC 90 | | Filtrate | Filtrate |
| | Polymer | Polymer % | (mg) | (mg) | Solution | (ml) | (g) |
| 41 | poly(sulfopropyl | 25% | 75 | 225 | 0% | 6.4 | 6.50 |
| | acrylate, potassium) | | | | ETOH | | |
| 42 | poly(sulfopropyl | 25% | 75 | 225 | 10% | 6.5 | 6.52 |
| | acrylate, potassium) | | | | ETOH | | |
| 43 | poly(sulfopropyl | 25% | 75 | 225 | 20% | 6.5 | 6.41 |
| | acrylate, potassium) | | | | ETOH | | |
| 44 | poly(sulfopropyl | 25% | 75 | 225 | 40% | 6.4 | 6.19 |
| | acrylate, potassium) | | | | ETOH | | |
| 45 | poly(sulfopropyl | 25% | 75 | 225 | 60% | 6.7 | 6.16 |
| | acrylate, potassium) | | | | ETOH | | |
| 46 | poly(sulfopropyl | 25% | 75 | 225 | 80% | 7.3 | 6.28 |
| | acrylate, potassium) | | | | ETOH | | |
| 47 | poly(sulfopropyl | 25% | 75 | 225 | 100% | 8.7 | 6.95 |
| | acrylate, potassium) | | | | ETOH | | |
| 48 | poly(sulfopropyl | 25% | 75 | 210 | 40% | 5.9 | 5.63 |
| | acrylate, | | | | ETOH | | |
| | potassium) + PEO(5%) | | | | | | |
| 49 | poly(sulfopropyl | 25% | 75 | 210 | 0% | 6 | 6.11 |
| | acrylate, | | | | ETOH | | |
| | potassium) + PEO(5%) | | | | | | |

Filterability of compositions (300 mg) containing Prosolv and crosslinked polyacrylamide in different solutions:

| | | | | Prosolv | | | |
|---|---|---|---|---|---|---|---|
| | | | Polymer | SMCC 90 | | Filtrate | Filtrate |
| | Polymer | Polymer % | (mg) | (mg) | Solution | (ml) | (g) |
| 50 | polyacrylamide | 25% | 75 | 225 | H₂O | 0 | 0.00 |
| 51 | polyacrylamide | 25% | 75 | 225 | 5% ETOH | 0 | 0.00 |
| 52 | polyacrylamide | 25% | 75 | 225 | 10% ETOH | 0 | 0.00 |
| 53 | polyacrylamide | 25% | 75 | 225 | 20% ETOH | 0 | 0.00 |
| 54 | polyacrylamide | 25% | 75 | 225 | 20% ETOH | 0 | 0.00 |
| 55 | polyacrylamide | 25% | 75 | 225 | 30% ETOH | 0.5 | 0.52 |
| 56 | polyacrylamide | 25% | 75 | 225 | 40% ETOH | 0.85 | 0.81 |
| 57 | polyacrylamide | 25% | 75 | 225 | 40% ETOH | 2 | 1.93 |
| 58 | polyacrylamide | 25% | 75 | 225 | 50% ETOH | 3.3 | 3.19 |
| 59 | polyacrylamide | 25% | 75 | 225 | 60% ETOH | 8 | 7.27 |
| 60 | polyacrylamide | 25% | 75 | 225 | 70% ETOH | 9 | 7.99 |
| 61 | polyacrylamide | 25% | 75 | 225 | 80% ETOH | 8.6 | 7.48 |
| 62 | polyacrylamide | 25% | 75 | 225 | 100% | 8.9 | 7.09 |
| | | | | | ETOH | | |
| 63 | polyacrylamide | 25% | 75 | 225 | 0.9% NaCl | 6.4 | 6.56 |
| 64 | polyacrylamide | 50% | 150 | 150 | 80% ETOH | 8.9 | 7.64 |

Filterability of compositions (300 mg) containing Prosolv, crosslinked polyacrylamide, and polyethylene oxide in different solutions:

| | | | | Prosolv | | | |
|---|---|---|---|---|---|---|---|
| | | | | SMCC | | | |
| | | | Polymer | 90 | | Filtrate | Filtrate |
| | Polymer | Polymer % | (mg) | (mg) | Solution | (ml) | (g) |
| 65 | polyacrylamide + PEO(5%) | 25% | 75 | 210 | 40% | 0.75 | 0.76 |
| | | | | | ETOH | | |
| 66 | polyacrylamide + PEO(5%) | 25% | 75 | 210 | 60% | 7.7 | 7.06 |
| | | | | | ETOH | | |
| 67 | polyacrylamide + PEO(5%) | 25% | 75 | 210 | 80% | 7.2 | 6.21 |
| | | | | | ETOH | | |
| 68 | polyacrylamide + PEO(5%) | 25% | 75 | 210 | 100% | 8.9 | 7.09 |
| | | | | | ETOH | | |

Filterability of compositions containing (300 mg) Prosolv and crosslinked poly(acrylamide-co-sodium acrylate) in different solutions:

| | | | | Prosolv | | | |
|---|---|---|---|---|---|---|---|
| | | | Polymer | SMCC 90 | | Filtrate | Filtrate |
| | Polymer | Polymer % | (mg) | (mg) | Solution | (ml) | (g) |
| 69 | poly(acrylamide-co-sodium | 25% | 75 | 225 | 0% | 0 | 0.00 |
| | acrylate) | | | | ETOH | | |
| 70 | poly(acrylamide-co-sodium | 25% | 75 | 225 | 10% | 0 | 0.00 |
| | acrylate) | | | | ETOH | | |
| 71 | poly(acrylamide-co-sodium | 25% | 75 | 225 | 20% | 0.5 | 0.56 |
| | acrylate) | | | | ETOH | | |
| 72 | poly(acrylamide-co-sodium | 25% | 75 | 225 | 30% | 1.1 | 1.12 |
| | acrylate) | | | | ETOH | | |
| 73 | poly(acrylamide-co-sodium | 25% | 75 | 225 | 40% | 4.2 | 4.05 |
| | acrylate) | | | | ETOH | | |
| 74 | poly(acrylamide-co-sodium | 25% | 75 | 225 | 50% | 5.3 | 4.95 |
| | acrylate) | | | | ETOH | | |
| 75 | poly(acrylamide-co-sodium | 25% | 75 | 225 | 60% | 8.1 | 7.37 |
| | acrylate) | | | | ETOH | | |
| 76 | poly(acrylamide-co-sodium | 25% | 75 | 225 | 80% | 8.7 | 7.49 |
| | acrylate) | | | | ETOH | | |
| 77 | poly(acrylamide-co-sodium | 25% | 75 | 225 | 100% | 8.8 | 6.97 |
| | acrylate) | | | | ETOH | | |

Compositions Containing Tramadol HCl, Superabsorbent and Plastic Agent

| Composition | Formula | | | | |
|---|---|---|---|---|---|
| (mg) | 78 | 79 | 80* | 81 | 82* |
| Tramadol HCl | 25 | 25 | 25 | 25 | 25 |
| Prosolv SMCC | 275 | 200 | 140 | 200 | 140 |
| 90 | | | | | |
| Crosslinked | | 75 | 75 | | |
| poly(sodium | | | | | |
| acrylate) | | | | | |
| Crosslinked | | | | 75 | 75 |
| polyacrylamide | | | | | |
| Kollidon SR** | | | 60 | | 60 |
| TOTAL (mg) | 300 | 300 | 300 | 300 | 300 |

*Subjected to a 120° C. dry heat curing process for 30 min post tableting;
**8:2 physical blend of poly(vinyl acetate) and polyvinylpyrrolidone.

Extract from Crushed Tablets

Liquid extraction: Each tablet was first crushed by placing into a Wedgewood mortar and then hammering down on the tablet with the pestle till the tablet was visibly cracked. Next, the broken tablet was triturated in a clock-wise motion for 10 revolutions to further grind to a powder. 10 ml of water was then added to the resultant particles and left to stand alone for two minutes. After the completion of this step, the extract mixture was poured into a glass funnel previously lined with Abaca fiber tea filter (Perfectea Filter™, Teavana) and the resultant liquid was collected and measured for total volume and total drug concentration in the extracted medium analyzed by UV absorbance @271 nm using Shimadzu UV-1700).

| | Volume extracted | % of Tramadol |
|---|---|---|
| Formula | (ml) | recovered |
| 78 | 9 | 76.4 |
| 79 | 2.2 | 8.5 |
| 80 | 3.3 | 9.9 |
| 81 | 1 | 3.5 |
| 82 | 2.2 | 4.3 |

Tablet Dissolution

Dissolution profiles were obtained using a USP 2 Paddle method in 900 ml of 0.1N HCl at 37.5° C. at a paddle rotational speed of 50 rpm.

| Time | mg of drug released | | | % of Tramadol released | | |
|---|---|---|---|---|---|---|
| (min) | 78 | 79 | 81 | 78 | 79 | 81 |
| 0 | 0 | 0 | 0 | 0 | 0 | 0 |
| 10 | 19.98 | 27.68727 | 26.57455 | 79.92 | 110.7491 | 106.2982 |
| 20 | 23.7436364 | 26.41091 | 25.51091 | 94.97455 | 105.6436 | 102.0436 |
| 30 | 25.2818182 | 27.14727 | 25.42909 | 101.1273 | 108.5891 | 101.7164 |

Bilayer Matrix Tablets:

A Bilayer Tablet Containing 50 wt % Deterrence Layer

Composition preparation: Crosslinked sodium salt of acrylic acid (swelling capacity of 400-500 g/g in distilled water, sieved into different particle sizes, >500, >250, and >125 μm); and Silicified microcrystalline cellulose, Prosolv SMCC 90 (with an average particle size of 110 μm), used with no further sieving.

Tablet Manufacturing:

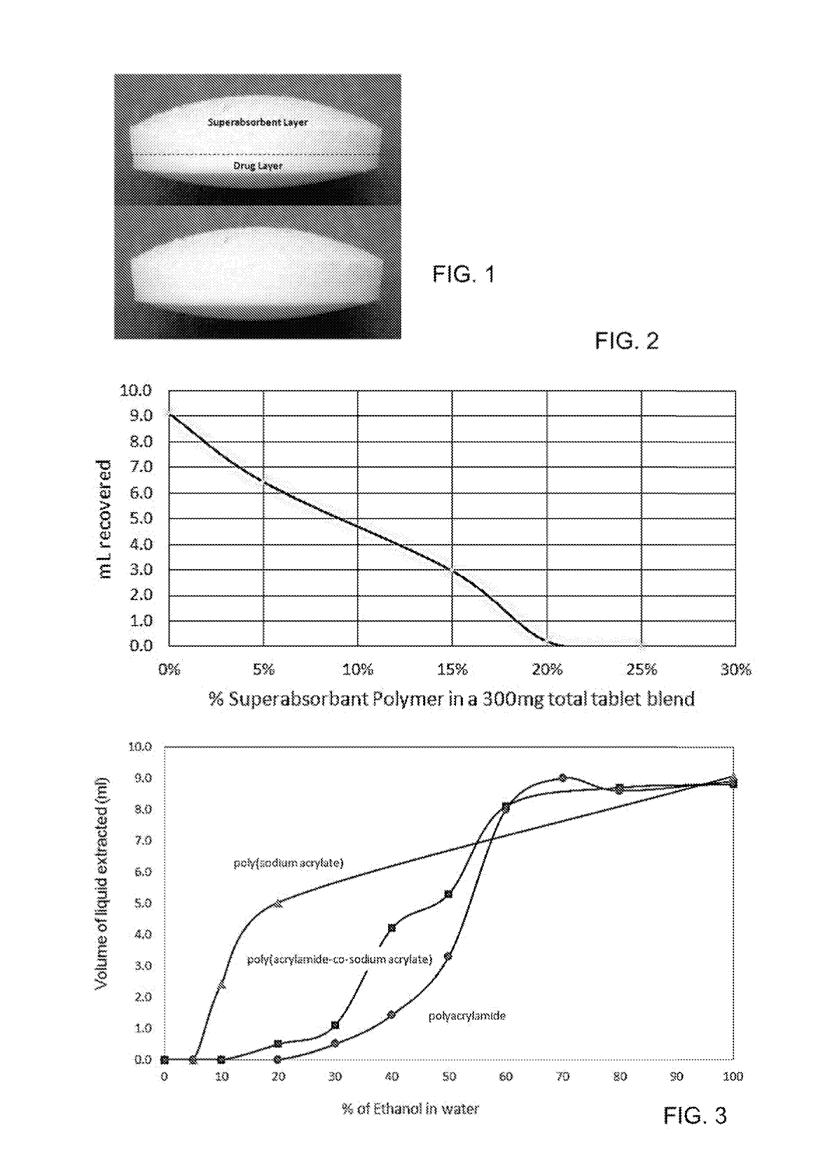

Total tablet weight was 350 mg. Each tablet contained 175 mg of Prosolv SMCC 90 and 175 mg of SAP (except control tablet). Control tablet was 350 mg of Prosolv SMCC 90. A rotary tablet press having a tablet die of 7/16″ was first filled with 350 mg of Prosolv, and manually turned a complete rotation to form a single layer tablet. A rotary tablet press having a tablet die of 7/16″ was first filled with 175 mg of Prosolv and manually turned to half compression and then rotated back. 175 mg of the SAP was then weighted and placed on top of the partially compressed Prosolv, and the rotary table manually turned a full rotation to form the bilayer tablet. Tablets were weighted after tableting and diameter and thickness measured using a digital micrometer. An illustrative tablet is shown in FIG. 1.

Whole tablets and crushed tablets were examined for gelation, and filterability studies according to the following procedure:

Whole tablets: Using a video camera (MightyScope microviewer), each tablet was visually inspected for its behavior in the presence of 10 mL of water. 1) Tablets were stored in a desiccator (RH 35-40%) for at least 24 hours prior testing, 2) Tablets were placed with (abuse deterrent layer, ADL) face up toward the camera in the center of a 50 ml pyrex beaker, 3) 10 mL of Millipore water was then added to the beaker (using a 30 ml syringe to measure out water, 4) With no stirring or mixing, video image was captured and the time it took to start seeing a gel (gelation period) was noted, 5) The beaker was then turned over to determine if the resultant gel was flowable.

Crushed tablets: Each tablet was crushed and then visually inspected using a video camera (MightyScope microviewer) for its behavior in the presence of 10 mL of water. 1) Tablets were stored in a desiccator (RH 35-40%) for at least 24 hours prior to testing, 2) Each tablet was hand broken into quarters and then placed into a glass mortar and triturated for 50 revolutions in a clockwise concentric circular motion, 3) Once crushed, 10 mL of Millipore water was then measured out using a 30 mL syringe and added to the mortar. The water was dripped over the pestle and into the mortar to gather any remaining powered that remained that was not captured during manual scraping into the mortar, 4) The mixture was visually inspected and the gelation period was noted.

Optimum concentration of primary superabsorbent polymer, poly(sodium acrylate):

Based on the graph in FIG. 2, an oral tablet comprising 20 wt % of the polymer will absorb all 10 mL of deionized water used for the extraction purpose.

Application range of different primary superabsorbent polymers in various hydro-alcoholic solutions:

Three tablets comprising 25 wt % of poly(sodium acrylate), polyacrylamide, and poly(acrylamide-co-sodium acrylate) were prepared and their crushed particles were added into 10 mL of different hydro-alcoholic solutions (0-100 v/v % ethanol).

With reference to FIG. 3, tablets prepared with poly(sodium acrylate) started to lose their ultimate swelling and deterrence capacity in hydroalcoholic solutions with ethanol concentrations greater than 5 v/v %. In 20 v/v % ethanol solution, the tablets could still absorb 50% of the solution. Tablets prepared by polyacrylamide, on the other hand, started to lose their ultimate swelling and deterrence capacity in solutions containing over 20 v/v % alcohol. However the rate of losing swelling and deterrence capacity for these polymers is much slower than with poly(sodium acrylate). For instance, such tablets can still absorb 50% of the extracting solutions containing over 50 v/v % ethanol.

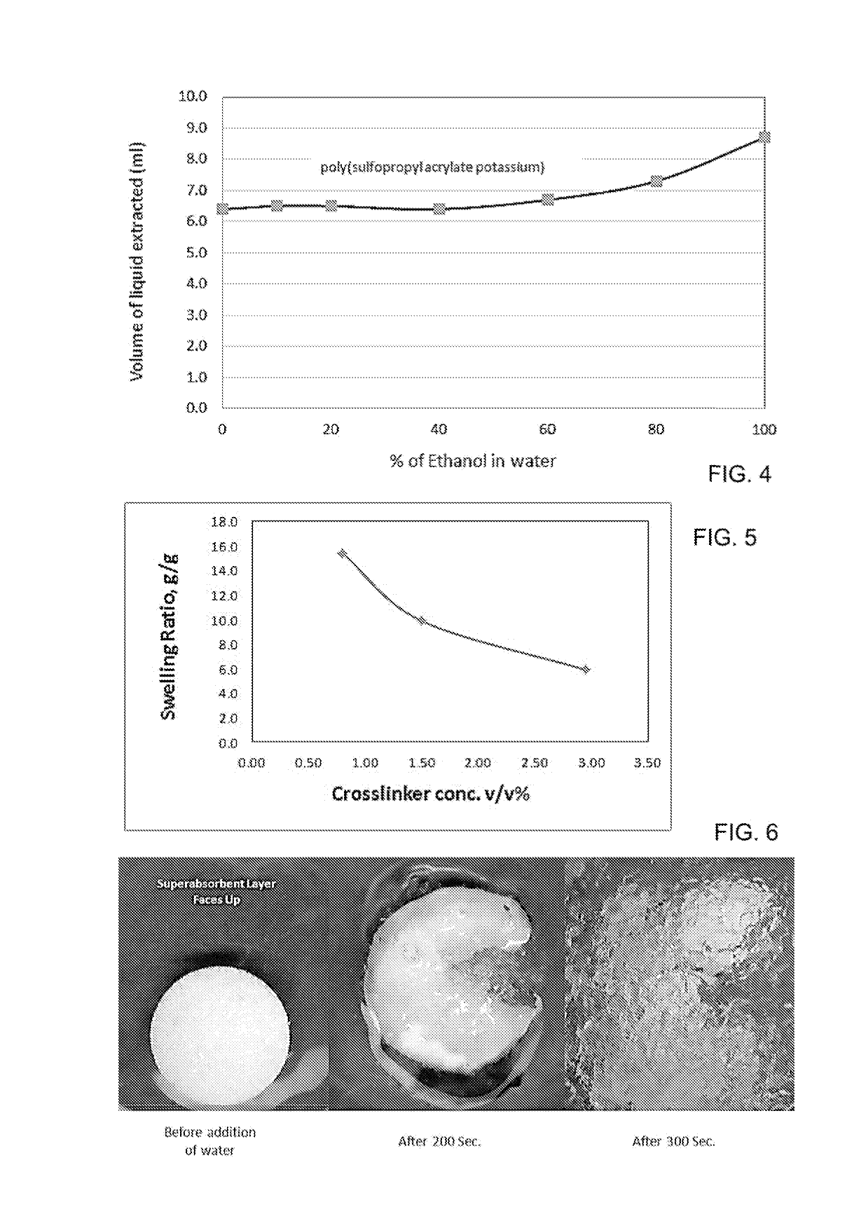

With reference to FIG. 4, a primary superabsorbent polymer with very high alcohol tolerance: While a reasonably high alcohol tolerance can be achieved with tablets containing polyacrylamide, poly(sulfopropyl acrylate potassium) could provide the maximum ethanol tolerance. Tablets containing this polymer started to lose their ultimate swelling and deterrence capacity in solutions containing over 65 v/v % ethanol. Moreover, the rate of losing the swelling and deterrence capacity beyond this point (>65 v/v ethanol) was very slow. The graph in FIG. 4 shows that tablets containing 25 wt % of this polymer can absorb only 3.5 mL of the extracting solution, and it may sound opposite to what aforementioned about the unique tolerance capacity of this polymer. The tolerance capacity is defined by the change or transition in the amount of the extractable liquid, and this will not occur with this polymer until a hydroalcoholic solution containing 65 v/v % of ethanol is used for extraction. However the maximum or ultimate swelling capacity is not determined by alcohol concentration, it's determined instead by the amount of crosslinker in the polymer formulation.

With reference to FIG. 5, the polymer used for this study is a highly crosslinked polymer, the lower the crosslinker concentration, the greater the ultimate swelling capacity. The following data shows how different crosslinked poly(sulfopropyl acrylate potassium) polymers prepared at different crosslinker concentrations behave differently in 20 v/v % alcohol solution. The polymer has been prepared using 2 mL of monomer solution (aq, 50 wt %), poly(ethylene glycol diacrylate), 0.3 mL of tetramethylethylenediamine (aq, 10 v/v %), and 0.16 mL of ammonium persulfate (aq, 10 wt %).

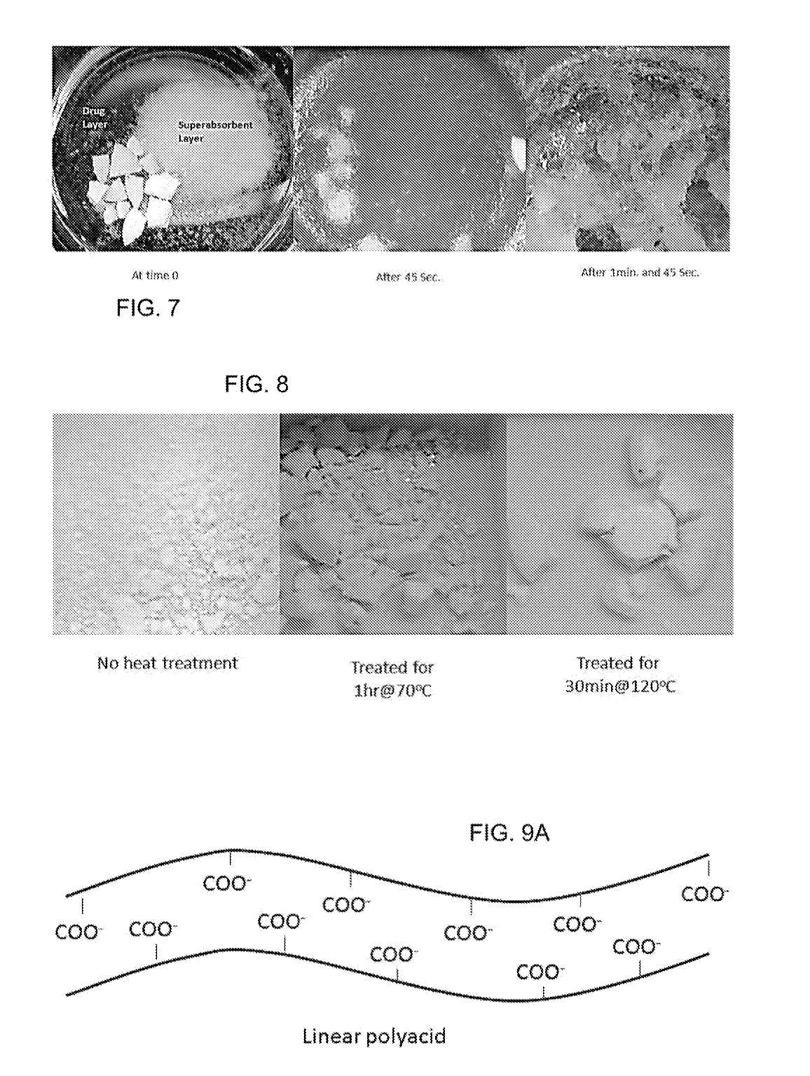

FIG. 6 illustrates an effect of the superabsorbent polymer on extracting solution (whole tablet in the extracting medium). FIG. 7 illustrates an effect of the superabsorbent polymer on extracting solution (crushed tablet in the extracting medium). FIG. 8 illustrates an effect of the use of plastic agent and the heat treatment on tablet crushability.

Solvent Volume Extract from Crushed Tablets

Solvent volume extraction: Each tablet composition formulation was placed into a glass mortar and 10 mL of extraction solvent was then added and left for two minutes. After the completion of this step, the extract mixture was poured into a glass funnel previously lined with Abaca fiber tea filter (Perfectea Filter™, Teavana) and the resultant liquid was collected and measured for total recoverable volume.

Compositions Containing Superabsorbent

| | Formula | | | |
|---|---|---|---|---|
| | 83 | 84 | 85 | 86 |
| Prosolv SMCC 90 | 225 | 225 | 225 | 225 |
| Crosslinked poly(sodium acrylate) | | | 75 | |
| Crosslinked polyacrylamide | 75 | | | |
| poly(acrylamide-co-potassium acrylate) | | 75 | | |
| TOTAL (mg) | 300 | 300 | 300 | 225 |

| | Extraction | Volume extracted |
|---|---|---|
| Formula | Solution | (ml) |
| 83 | Water | 0.0 |
| 84 | Water | 0.0 |
| 85 | Water | 0.0 |
| 86 | Water | 9.7 |
| 83 | 40% EtOH | 2.0 |
| 84 | 40% EtOH | 3.3 |
| 85 | 40% EtOH | 8.9 |
| 86 | 40% EtOH | 9.1 |
| 83 | 0.9% NaCl | 6.7 |
| 84 | 0.9% NaCl | 6.7 |
| 85 | 0.9% NaCl | 6.7 |
| 86 | 0.9% NaCl | 9.5 |
| 83 | 0.1N HCl | 8.6 |
| 84 | 0.1N HCl | 8.7 |
| 85 | 0.1N HCl | 8.2 |
| 86 | 0.1N HCl | |

Tablets may be prepared as described above.

Drug release profile of matrix tablets containing Tramadol HCl and both low and high amounts of SAP.

Materials:

Crosslinked polyacrylamide (Hydrosource CLP, about 250 μm), crosslinked sodium salt of acrylic acid (Waste Lock 770, about 250 μm), and silicified microcrystalline cellulose (Prosolv SMCC 90, 110 μm), and Tramadol HCl.

Methods:

Tablet manufacturing: Matrix tablets were made on a single station carver press at a compression force of approximately 1000 pounds using a 7/16″ punch and die. Tablets were then subjected to dissolution studies using a USP 2 Paddle method in 900 mL of 0.1 N HCl at 37.5° C. with a paddle rotational speed of 50 rpm. Tramadol HCl concentration in the dissolution medium was analyzed over time. Tablet compositions were made in triplicate as follows:

| | | | | | | Calculated |
|---|---|---|---|---|---|---|
| | | | | | Prosolv | tablet |
| | Tablet | | Tramadol | SAP | SMCC 90 | weight |
| SAP Polymer | ID | SAP/Tramadol | HCl (mg) | (mg) | (mg) | (mg) |
| Blank | BL-low | 0:0 | 25 | 0 | 175 | 200 |
| | BL-high | 0:0 | 25 | 0 | 375 | 400 |
| Polyacrylamide | HS-low | 3:1 | 25 | 75 | 100 | 200 |
| | HS- | 8:1 | 25 | 200 | 175 | 400 |
| | high | | | | | |
| Poly(sodium acrylate) | WL-low | 3:1 | 25 | 75 | 100 | 200 |
| | WL- | 8:1 | 25 | 200 | 175 | 400 |
| | high | | | | | |

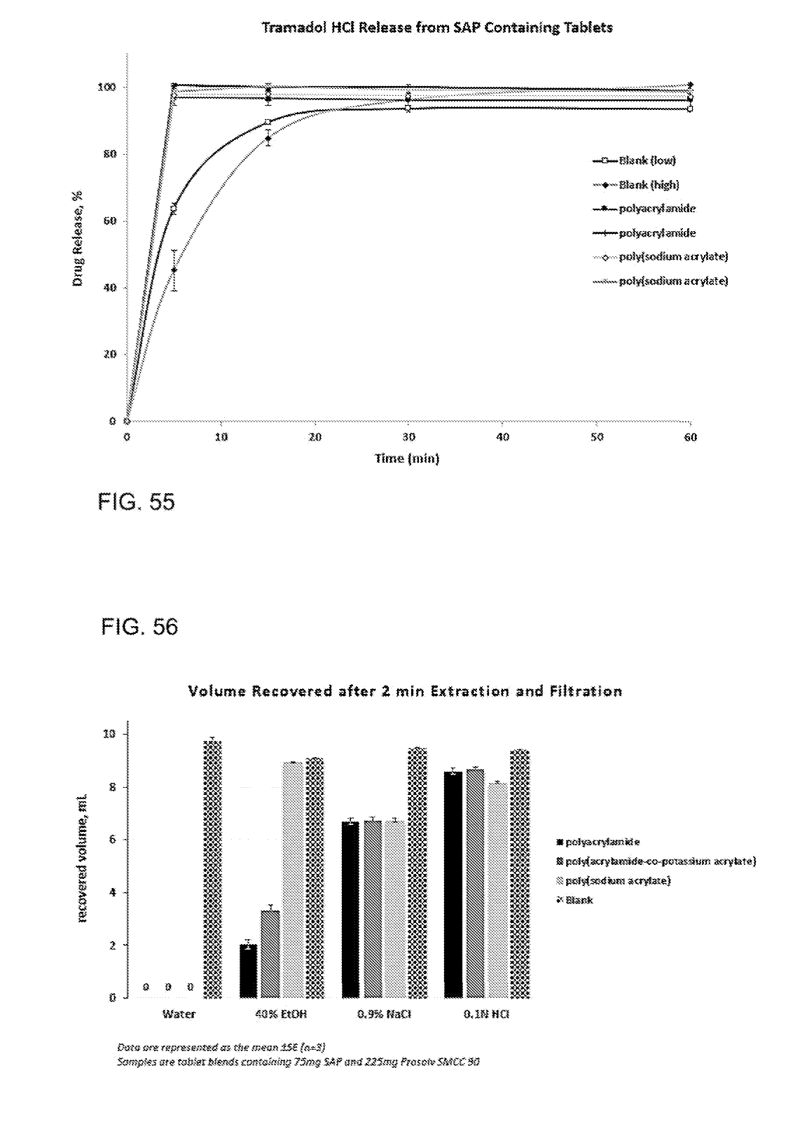

FIG. 55 illustrates Tramadol release from SAP tablets containing low and high concentrations of either polyacrylamide or poly(sodium acrylate). The data show that Tramadol release is not affected by either the type of superabsorbent or its concentration in the tablet.

FIG. 56 illustrates the amount of extraction volume recovery for control tablet and tablets containing polyacrylamide, poly(sodium acrylate) or their copolymer. The data show tablet containing homo or copolymers of acrylamide resist the 40% EtOH solution the most.

The disclosure describes the use of certain pharmaceutically acceptable functional polymers that are used to make more effective abuse deterrent medications. This disclosure describes different approaches that can potentially deter abuse by reducing the efficacy of main processes utilized by abusers to speed drug absorption and enhance its effect. An alternative embodiment of the disclosure will now be described.

Polymers

A first primary polymer is an internally crosslinked polymer based on natural, synthetic or semi-synthetic materials carrying accessible acidic groups, and is insoluble in water. The second primary polymer is a linear polyacid polymer based on the same material without being crosslinked throughout the process of manufacturing. It may carry the same functionality as the first primary polymer, and is water soluble. The polyacid polymer may be either internally crosslinked or chemically crosslinked.

Crushing

Primary polymers of the disclosure can effectively bind to the drug via their binding sites, and their binding remains stable over a wide variety of abuse conditions as outlined in this disclosure.

In an embodiment, polyacid polymers are mixed with an aqueous solution of the drug (e.g., Tramadol HCl), and the mixture is vacuum-dried at low temperature. The dried drug-polyacid complex is then used in the preparation of a tablet. Since the drug is not free and already bound to the structure of the polyacid, the drug will not be easily released if the abusers sniff the crushed tablet.

Abuse

The tablet will contain an ionic drug (e.g., Tramadol HCl), a polyacid (deterrent agent), and other necessary excipients required to prepare the tablet dosage form. Once in solution, the polyacid will immediately form a strong complex with the basic drug, and prevents the abusable drug from being extracted into solution. The drug-polyacid complex will break apart in the strong acidic medium of the stomach when patients take the drug as prescribed.

Alcohol Co-Ingestion

The polyacid-drug complex of this disclosure will resist hydroalcoholic solutions over an applicable range of alcohol concentrations commonly used in the abuse process.

The abusers may use the whole tablet with an ingestion of alcohol. The primary polymers of this disclosure can effectively bind to the drug via their binding sites, and their binding remains stable over a wide variety of abuse conditions as outlined in this disclosure. The primary polymers of this disclosure will not change the release profile of the active ingredient in the acidic environment of the stomach as intended for regular patient.

Polymer Features

Polyacids of this embodiment can advantageously possess the characteristics of being synthetic, natural or semi-synthetic; either linear or crosslinked; if crosslinked, they are chemically crosslinked using internal crosslinking or via addition of a chemical crosslinker; and the crosslinked polymer should have its acid groups freely accessible to weak bases. Since physical crosslinking involves the addition of metal ions, and metal ions consume acid groups of the polyacid in an uncontrollable fashion, physically crosslinked polyacids may not provide abuse-deterrence.

Both linear and crosslinked polymers can be utilized in abuse-deterrent preparation according to this disclosure. The polyacid-drug binding should be effective under abuse conditions, and become ineffective under regular administration of the abusable composition.

Polyacids can either be physically mixed with the drug during the dosage form preparation, or their complex with the abusable drug may be used during the dosage form preparation.

Non-limiting examples of such polymers include linear and crosslinked sodium carboxymethylcellulose, linear and crosslinked sodium carboxymethyl starch, linear and crosslinked polyacrylate salts (sodium, potassium, and ammonium), linear and crosslinked polymethacrylate salts (sodium, potassium, and ammonium), linear and crosslinked poly(potassium sulfopropyl acrylate), linear and crosslinked poly(2-acrylamido 2-methyl 1-propane sulfonic acid (AMPS)).

Synthetic polymers of this disclosure can be prepared following a general experimental procedure that we previously reported [26-29] which are incorporated herein by reference, or their purified commercial counterparts can be used instead.

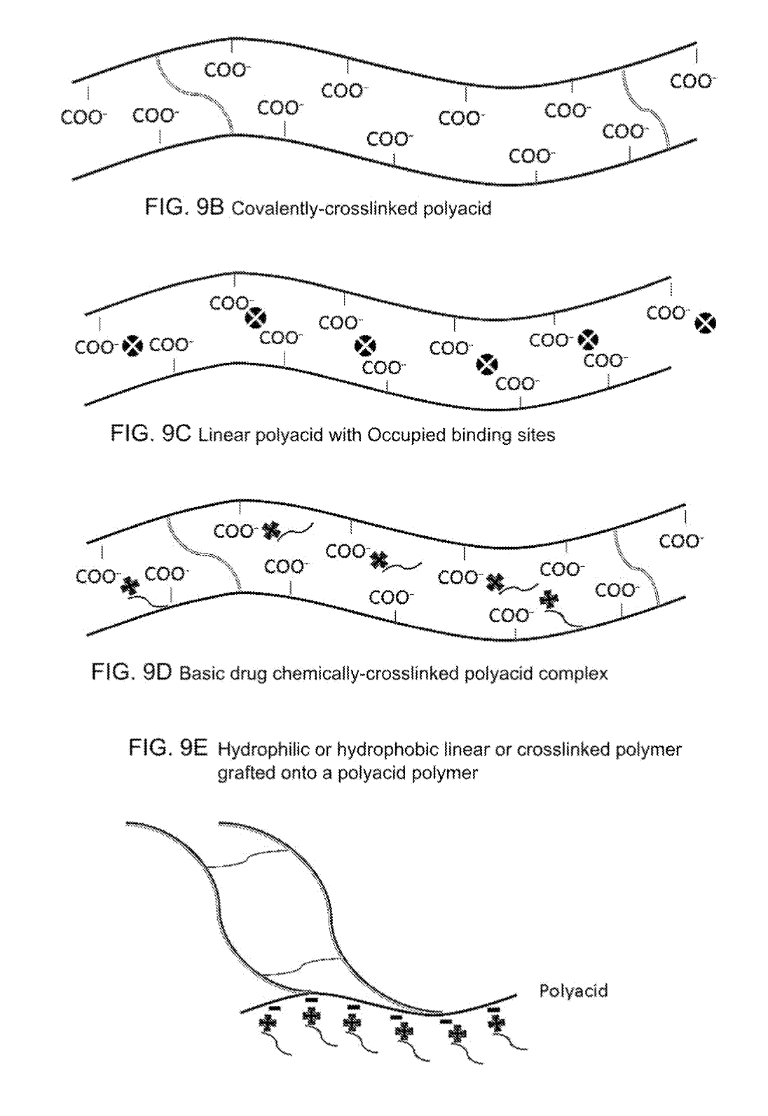

The schemes in FIGS. 9A to 9E depict linear and crosslinked polyacids of this disclosure. FIG. 9E depicts a graft copolymer of a polyacid with another hydrophilic of hydrophobic polymer in the form of semi-interpenetrated or fully-interpenetrated network.

### Examples

The following elucidate the binding mechanism and release capacity of the dosage forms containing a deterrent agent of this disclosure and Tramadol HCl.

IC-SCMC Polyacid—Internally Crosslinked Sodium Carboxymethyl Cellulose is a water-swellable cellulose-based polyacid carrying free carboxyl groups susceptible to bind to a positively charged drug such as Tramadol HCl. The polymer is internally crosslinked without using an external bi- or polyfunctional crosslinker. Ac-Di-Sol® (FMC Corporation) is an internally-crosslinked sodium salt of carboxymethylcellulose, commonly used as superdisintegrant in immediate release solid pharmaceutical compositions, and evaluated in this study. The purpose of this study was to show that IC-SCMC is extremely capable of entrapping weak basic drugs under abuse conditions, and is extremely capable of releasing the drug when administered as prescribed.

IC-SCMS Polyacid—Internally Crosslinked Sodium Carboxymethyl Starch is a water-swellable starch-based polyacid carrying free carboxyl groups susceptible to bind to a positively charged drug such as Tramadol HCl. The polymer is internally crosslinked without using an external bi- or polyfunctional crosslinker. IC-SCMS has less available carboxyl groups than IC-SCMC. Explotab® (JRS Pharma) is an internally crosslinked sodium salt of carboxymethyl starch, commonly used in immediate release pharmaceutical compositions, and evaluated in this study. The purpose of this study was to confirm the results obtained in the study with IC-SCMC, and to show that different deterrent capacity is related to different levels of binding sites available in the polymer structure.

PC-SCMC Polyacid SCMC physically crosslinked with calcium aluminum cation blends is a water soluble sodium carboxymethyl cellulose was physically crosslinked with different cation blends comprising aluminum and calcium. The purpose of this study was to show that not all crosslinked carboxymethylcellulose materials possess deterrence capacity. A mixture of calcium and aluminum cations can bind into free carboxyl groups of the CMC, and will make them inactive for abuse-deterrence applications.

IC-PVP (non-acid)—Internally Crosslinked Polyvinyl Pyrrolidone is a water swellable non-ionic internally crosslinked polymer based on vinylpyrrolidone, which is commonly used as superdisintegrant in immediate release pharmaceutical compositions. Polyplasdone XL® (BASF) was used in this study to confirm that an internally crosslinked water-swellable polymer with no binding sites is not capable of entrapping weak basic drugs, and hence it's not abuse-deterrent.

IC-SCMC Polyacid

Effect of Concentration

A 10 ml volume of 25 μg/ml Tramadol HCl aqueous solution was added to different weights of IC-SCMC. Samples were vortexed for 5 sec, and then centrifuged at 1500 rpm for 5 min. Supernatant was then analyzed for Tramadol concentration using UV-Visible Spectroscopy (UV-1700, Shimadzu).

| Exam- | IC-SCMC | IC-SCMC, | Abs@ | Tramadol HCl, |
|---|---|---|---|---|
| ple | (mg) | mg/ml | 271 nm | μg/ml |
| 1 | 0 | 0 | 0.1434 | 25.38 |
| 2 | 2.5 | 0.25 | 0.0513 | 9.5 |
| 3 | 5 | 0.5 | 0.043 | 8.07 |
| 4 | 10 | 1 | 0.0277 | 5.43 |
| 5 | 20 | 2 | 0.0227 | 4.57 |
| 6 | 40 | 4 | 0.0194 | 4 |

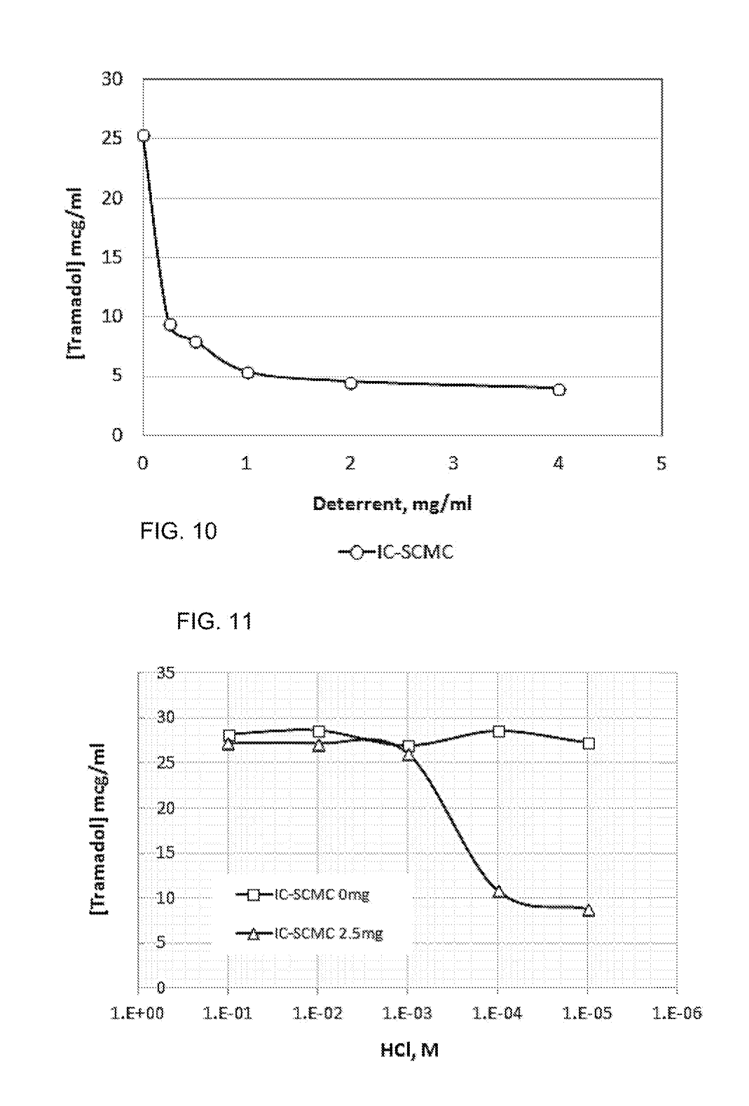

FIG. 10 illustrates IC-SCMC, over the concentrations range of 0-4 mg/ml, showing its strongest binding and entrapping potential at concentrations as low as 0.25 mg/ml.

Effect of pH

A 10 ml volume of 25 μg/ml Tramadol HCl aqueous solution made of different molar concentrations of HCl was added to different weights of IC-SCMC. Samples were vortexed for 5 sec, and then centrifuged at 1500 rpm for 5 min. Supernatant was then analyzed for Tramadol concentration using UV-Visible Spectroscopy (UV-1700, Shimadzu).

| | | Acidic | | | |
|---|---|---|---|---|---|
| | | Tramadol | | | |
| | | solution | | | |
| Exam- | IC-SCMC, | (25 μg/ml) | IC-SCMC | Abs@ | Tramadol HCl, |
| ple | mg | in HCl, M | mg/ml | 271 nm | μg/ml |
| 7 | 0 | 0.1 | 0 | 0.1563 | 28.18 |
| 8 | 2.5 | 0.1 | 0.25 | 0.1508 | 27.18 |
| 9 | 0 | 0.01 | 0 | 0.1587 | 28.62 |
| 10 | 2.5 | 0.01 | 0.25 | 0.1506 | 27.15 |
| 11 | 0 | 0.001 | 0 | 0.1493 | 26.91 |
| 12 | 2.5 | 0.001 | 0.25 | 0.1449 | 26.11 |
| 13 | 0 | 0.0001 | 0 | 0.1583 | 28.55 |
| 14 | 2.5 | 0.0001 | 0.25 | 0.0612 | 10.89 |
| 15 | 0 | 0.00001 | 0 | 0.1511 | 27.24 |
| 16 | 2.5 | 0.00001 | 0.25 | 0.0497 | 8.8 |

FIG. 11 illustrates that IC-SCMC will hold its binding with Tramadol down to pH 4, and its binding potential becomes completely ineffective below pH 3.

Effect of Thermal Treatment

A 10 ml volume of 25 μg/ml Tramadol HCl aqueous solution was added to different weights of IC-SCMC. Samples were vortexed for 5 sec, and then subjected to an 80° C. thermal treatment (water bath) for 5 minutes. After being removed, samples were then centrifuged at 1500 rpm for 5 min. Supernatant was then analyzed for Tramadol concentration using UV-Visible Spectroscopy (UV-1700, Shimadzu).

| Exam- | IC-SCMC, | IC-SCMC, | Abs@ | Tramadol HCl, |
|---|---|---|---|---|
| ple | mg | mg/ml | 271 nm | μg/ml |
| 17 | 0 | 0 | 0.1476 | 26.10 |
| 18 | 2.5 | 0.25 | 0.0323 | 6.22 |
| 19 | 5 | 0.5 | 0.0239 | 4.78 |
| 20 | 10 | 1 | 0.0192 | 3.97 |
| 21 | 20 | 2 | 0.0197 | 4.05 |
| 22 | 40 | 4 | 0.017 | 3.59 |

In Saline

A 10 ml volume of 25 μg/ml Tramadol HCl normal saline (0.9% NaCI) solution was added to different weights of IC-SCMC. Samples were vortexed for 5 sec, and then centrifuged at 1500 rpm for 5 min. Supernatant was then analyzed for Tramadol concentration using UV-Visible Spectroscopy (UV-1700, Shimadzu).

| Exam- | IC-SCMC, | IC-SCMC, | Abs@ | Tramadol HCl, |
|---|---|---|---|---|
| ple | mg | mg/ml | 271 nm | μg/ml |
| 23 | 0 | 0 | 0.0374 | 22.17 |
| 24 | 2.5 | 0.25 | 0.0349 | 20.78 |
| 25 | 5 | 0.5 | 0.0265 | 16.11 |
| 26 | 10 | 1 | 0.0251 | 15.33 |
| 27 | 20 | 2 | 0.0242 | 14.83 |
| 28 | 40 | 4 | 0.028 | 16.94 |

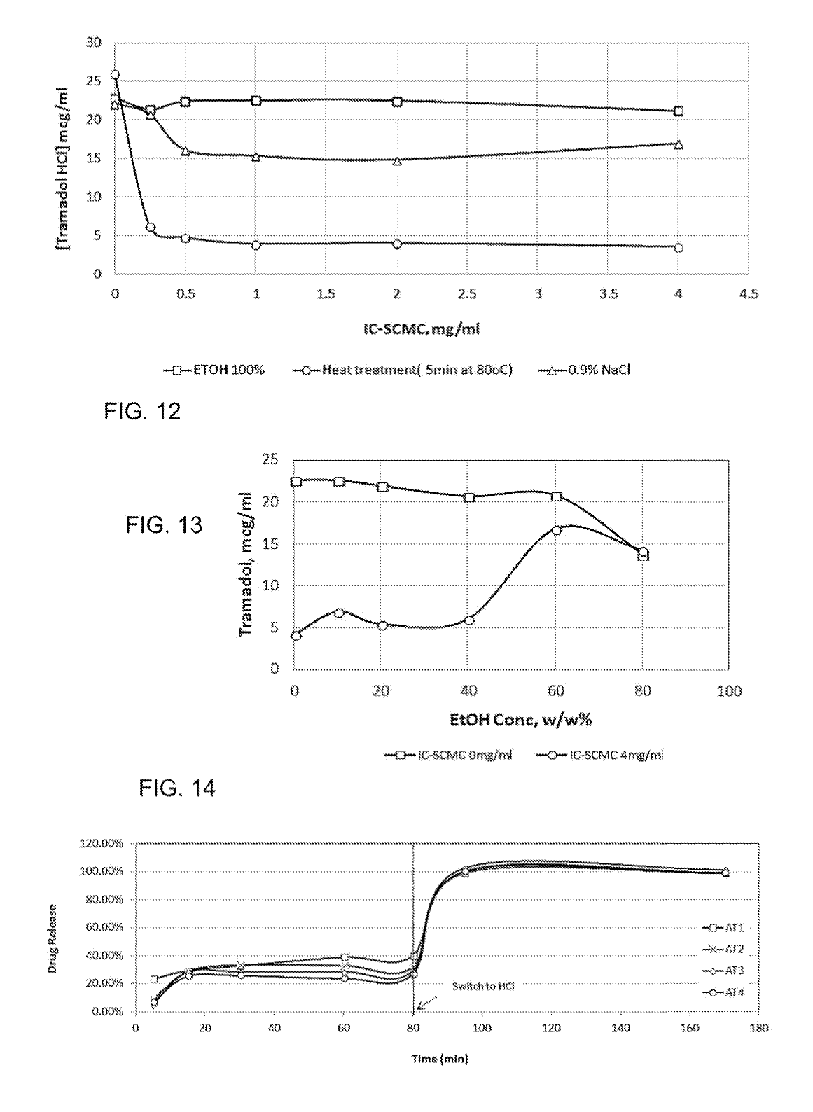

FIG. 12 illustrates that heating the drug solution containing IC-SCMC does not pose any negative effect on binding capacity of the deterrent agent. Pure EtOH completely deactivates the deterrence capacity of the deterrent agent, and 0.9% saline reduces the deterrence capacity down to almost 50%.

In Different Hydroalcoholic Solutions

A 10 ml volume of 25 μg/ml Tramadol HCl in various hydroalcoholic concentrations was added to different weights of IC-SCMC. Samples were vortexed for 5 sec, and then centrifuged at 1500 rpm for 5 min. Supernatant was then analyzed for Tramadol concentration using UV-Visible Spectroscopy (UV-1700, Shimadzu).

| Exam- | IC-SCMC, | EtOH Conc., | IC-SCMC, | Abs@ | Tramadol HCl, |
|---|---|---|---|---|---|
| ple | mg | w/w (aq) | mg/ml | 271 nm | μg/ml |
| 35 | 0 | 0 | 0 | 0.1497 | 22.66 |
| 36 | 40 | 0 | 4 | 0.024 | 4.18 |
| 37 | 0 | 10 | 0 | 0.1494 | 22.62 |
| 38 | 40 | 10 | 4 | 0.0428 | 6.94 |
| 39 | 0 | 20 | 0 | 0.145 | 21.97 |
| 40 | 40 | 20 | 4 | 0.0326 | 5.44 |
| 41 | 0 | 40 | 0 | 0.1365 | 20.72 |
| 42 | 40 | 40 | 4 | 0.0366 | 6.03 |
| 43 | 0 | 60 | 0 | 0.1375 | 20.87 |
| 44 | 40 | 60 | 4 | 0.11 | 16.82 |
| 45 | 0 | 80 | 0 | 0.09 | 13.88 |
| 46 | 40 | 80 | 4 | 0.0931 | 14.34 |

FIG. 13 illustrates that hydroalcoholic solutions containing up to 40 wt % EtOH do not affect the binding capacity of the IC-SCMC with Tramadol.

Tablet

IC-SCMC was formulated into tablets using four different formulas. Tablets were made on a single station carver press at a compression force of approximately 1000 pounds using a 7/16″ punch and die. Tablets were then subjected to dissolution studies using a USP 2 Paddle method (Distek dissolution system 2100A) in 900 ml of ultrapure water at 37.5° C. at a paddle rotational speed of 50 rpm. After 80 minutes, the dissolution medium was changed to 0.1N HCl by adding concentrated hydrochloric acid into the dissolution medium. Tramadol HCl concentration in the dissolution medium was analyzed using UV-visible Spectroscopy (UV-1700, Shimadzu) over time.

| | IC- | Tramadol | | Prosolv | Calculated | |
|---|---|---|---|---|---|---|
| | SCMC:Tramadol | HCl, | IC-SCMC, | SMCC90, | weight, | Actual |
| Tablet | w/w | mg | mg | mg | mg | weight, mg |
| AT | 4:1 | 25 | 100 | 100 | 225 | 216 |
| AT2 | 8:1 | 25 | 200 | 0 | 225 | 217 |
| AT3 | 12:1 | 25 | 300 | 0 | 325 | 318 |
| AT4 | 16:1 | 25 | 400 | 0 | 425 | 415 |

Dissolution Data

| | | Dissolution | Time, | | Tramadol HCl, | Drug |
|---|---|---|---|---|---|---|
| Example | Tablet | Medium | min | Abs @271 nm | μg/ml | released, % |
| 47 | AT1 | Water | 5 | 0.0342 | 6.55 | 23.59 |
| 48 | AT1 | Water | 15 | 0.0436 | 8.17 | 29.42 |
| 49 | AT1 | Water | 30 | 0.0492 | 9.14 | 32.90 |
| 50 | AT1 | Water | 60 | 0.0592 | 10.86 | 39.10 |
| 51 | AT1 | Water | 80 | 0.0604 | 11.07 | 39.85 |
| 52 | AT1 | 0.1N HCl | 95 | 0.1593 | 27.51 | 99.03 |
| 53 | AT1 | 0.1N HCl | 170 | 0.1593 | 27.51 | 99.03 |
| 54 | AT2 | Water | 5 | 0.01 | 2.38 | 8.57 |
| 55 | AT2 | Water | 15 | 0.0421 | 7.91 | 28.49 |
| 56 | AT2 | Water | 30 | 0.0497 | 9.22 | 33.21 |
| 57 | AT2 | Water | 60 | 0.0492 | 9.14 | 32.90 |
| 58 | AT2 | Water | 80 | 0.0475 | 8.84 | 31.84 |
| 59 | AT2 | 0.1N HCl | 95 | 0.1609 | 27.79 | 100.04 |
| 60 | AT2 | 0.1N HCl | 170 | 0.1589 | 27.44 | 98.78 |
| 61 | AT3 | Water | 5 | 0.0045 | 1.43 | 5.15 |
| 62 | AT3 | Water | 15 | 0.0416 | 7.83 | 28.18 |
| 63 | AT3 | Water | 30 | 0.042 | 7.90 | 28.43 |
| 64 | AT3 | Water | 60 | 0.042 | 7.90 | 28.43 |
| 65 | AT3 | Water | 80 | 0.0427 | 8.02 | 28.86 |
| 66 | AT3 | 0.1N HCl | 95 | 0.165 | 28.51 | 102.63 |
| 67 | AT3 | 0.1N HCl | 170 | 0.163 | 28.16 | 101.37 |
| 68 | AT4 | Water | 5 | 0.0074 | 1.93 | 6.95 |
| 69 | AT4 | Water | 15 | 0.0369 | 7.02 | 25.26 |
| 70 | AT4 | Water | 30 | 0.0377 | 7.16 | 25.76 |
| 71 | AT4 | Water | 60 | 0.0344 | 6.59 | 23.71 |
| 72 | AT4 | Water | 80 | 0.0392 | 7.41 | 26.69 |
| 73 | AT4 | 0.1N HCl | 95 | 0.1615 | 27.89 | 100.42 |
| 74 | AT4 | 0.1N HCl | 170 | 0.1597 | 27.58 | 99.28 |

The foregoing data is illustrated in FIG. 14.

IC-SCMS Polyacid

Effect of Concentration

A 10 ml volume of 25 μg/ml Tramadol HCl aqueous solution was added to different weights of IC-SCMS. Samples were vortexed for 5 sec, and then centrifuged at 1500 rpm for 5 min. Supernatant was then analyzed for Tramadol concentration using UV-Visible Spectroscopy (UV-1700, Shimadzu).

| Exam- | IC-SCMS, | IC-SCMS, | Abs@ | Tramadol HCl, |
|---|---|---|---|---|
| ple | mg | mg/ml | 271 nm | μg/ml |
| 75 | 0 | 0 | 0.1414 | 25.03 |
| 76 | 2.5 | 0.25 | 0.0883 | 15.88 |
| 77 | 5 | 0.5 | 0.0842 | 15.17 |
| 78 | 10 | 1 | 0.0796 | 14.38 |
| 79 | 20 | 2 | 0.0787 | 14.22 |
| 80 | 40 | 4 | 0.0802 | 14.48 |

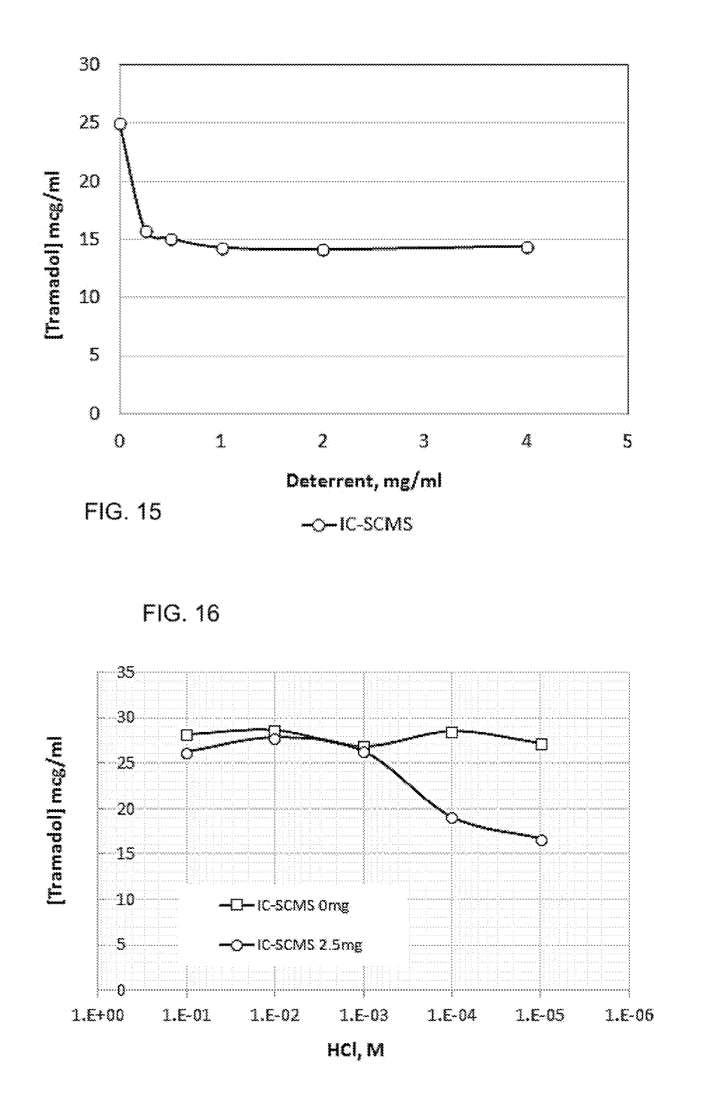

FIG. 15 illustrates that IC-SCMS, over the concentrations range of 0-4 mg/ml, shows its strongest binding and entrapping potential at concentrations as low as 0.25 mg/ml.

Effect of pH

A 10 ml volume of 25 μg/ml Tramadol HCl solution made at different molar concentrations of HCl, and was added to different weights of IC-SCMS. Samples were vortexed for 5 sec and then centrifuged at 1500 rpm for 5 min. Supernatant was then analyzed for Tramadol concentration using UV-Visible Spectroscopy (UV-1700, Shimadzu).

| | | Acidic | | | |
|---|---|---|---|---|---|
| | | Tramadol | | | |
| | | (25 μg/ml) | | | |
| Exam- | IC-SCMS, | HCl | IC-SCMS, | Abs@ | Tramadol HCl, |
| ple | mg | solution, M | mg/ml | 271 nm | μg/ml |
| 81 | 0 | 0.1 | 0 | 0.1563 | 28.18 |
| 82 | 2.5 | 0.1 | 0.25 | 0.1456 | 26.24 |
| 83 | 0 | 0.01 | 0 | 0.1589 | 28.65 |
| 84 | 2.5 | 0.01 | 0.25 | 0.1543 | 27.82 |
| 85 | 0 | 0.001 | 0 | 0.1493 | 26.91 |
| 86 | 2.5 | 0.001 | 0.25 | 0.146 | 26.31 |
| 87 | 0 | 0.0001 | 0 | 0.1583 | 28.55 |
| 88 | 2.5 | 0.0001 | 0.25 | 0.1064 | 19.11 |
| 89 | 0 | 0.00001 | 0 | 0.1511 | 27.24 |
| 90 | 2.5 | 0.00001 | 0.25 | 0.0934 | 16.75 |

FIG. 16 illustrates that IC-SCMC will hold its binding with Tramadol down to pH 4, and its binding potential becomes completely ineffective below pH 3.

Tablet

IC-SCMS was formulated into tablets using four different formulas. Tablets were made on a single station carver press at a compression force of approximately 1000 pounds using a 7/16″ punch and die. Tablets were then subjected to dissolution studies using a USP 2 Paddle method (Distek dissolution system 2100A) in 900 ml of ultrapure water at 37.5° C. at a paddle rotational speed of 50 rpm. After 80 minutes, the dissolution medium was changed to 0.1N HCl by adding concentrated hydrochloric acid into the dissolution medium. Tramadol HCl concentration in the dissolution medium was analyzed using UV-visible Spectroscopy (UV-1700, Shimadzu) overtime.

| | | Tramadol | | Prosolv | | |
|---|---|---|---|---|---|---|
| | | HCl, | IC-SCMS, | SMCC90, | Calculated | Actual |
| Tablet | IC-SCMS:Tramadol | mg | mg | mg | weight, mg | weight, mg |
| ET1 | 4:1 | 25 | 100 | 100 | 225 | 218 |
| ET2 | 8:1 | 25 | 200 | 0 | 225 | 219 |
| ET3 | 12:1 | 25 | 300 | 0 | 325 | 321 |
| ET4 | 16:1 | 25 | 400 | 0 | 425 | 416 |

Dissolution Data:

| | | | | | Tramadol | |
|---|---|---|---|---|---|---|
| | | Dissolution | Time, | | HCl, | Drug |
| Example | Tablet | Medium | min | Abs @271 nm | μg/ml | released, % |
| 91 | ET1 | Water | 5 | 0.0787 | 14.22 | 51.21 |
| 92 | ET1 | Water | 15 | 0.099 | 17.72 | 63.81 |
| 93 | ET1 | Water | 30 | 0.1063 | 18.98 | 68.34 |
| 94 | ET1 | Water | 60 | 0.1082 | 19.31 | 69.52 |
| 95 | ET1 | Water | 80 | 0.1066 | 19.03 | 68.52 |
| 96 | ET1 | 0.1N HCl | 95 | 0.1637 | 28.28 | 101.81 |
| 97 | ET1 | 0.1N HCl | 170 | 0.1627 | 28.11 | 101.18 |
| 98 | ET2 | Water | 5 | 0.0876 | 15.76 | 56.73 |
| 99 | ET2 | Water | 15 | 0.0935 | 16.78 | 60.39 |
| 100 | ET2 | Water | 30 | 0.0946 | 16.97 | 61.08 |
| 101 | ET2 | Water | 60 | 0.0945 | 16.95 | 61.01 |
| 102 | ET2 | Water | 80 | 0.0961 | 17.22 | 62.01 |
| 103 | ET2 | 0.1N HCl | 95 | 0.1563 | 26.98 | 97.14 |
| 104 | ET2 | 0.1N HCl | 170 | 0.1565 | 27.02 | 97.26 |
| 105 | ET3 | Water | 5 | 0.0778 | 14.07 | 50.65 |
| 106 | ET3 | Water | 15 | 0.0924 | 16.59 | 59.71 |
| 107 | ET3 | Water | 30 | 0.0934 | 16.76 | 60.33 |
| 108 | ET3 | Water | 60 | 0.0931 | 16.71 | 60.14 |
| 109 | ET3 | Water | 80 | 0.0945 | 16.95 | 61.01 |
| 110 | ET3 | 0.1N HCl | 95 | 0.1602 | 27.67 | 99.60 |
| 111 | ET3 | 0.1N HCl | 170 | 0.1575 | 27.19 | 97.89 |
| 112 | ET4 | Water | 5 | 0.0654 | 11.93 | 42.95 |
| 113 | ET4 | Water | 15 | 0.0885 | 15.91 | 57.29 |
| 114 | ET4 | Water | 30 | 0.088 | 15.83 | 56.98 |
| 115 | ET4 | Water | 60 | 0.0885 | 15.91 | 57.29 |
| 116 | ET4 | Water | 80 | 0.0889 | 15.98 | 57.54 |
| 117 | ET4 | 0.1N HCl | 95 | 0.1553 | 26.81 | 96.51 |
| 118 | ET4 | 0.1N HCl | 170 | 0.1531 | 26.42 | 95.12 |

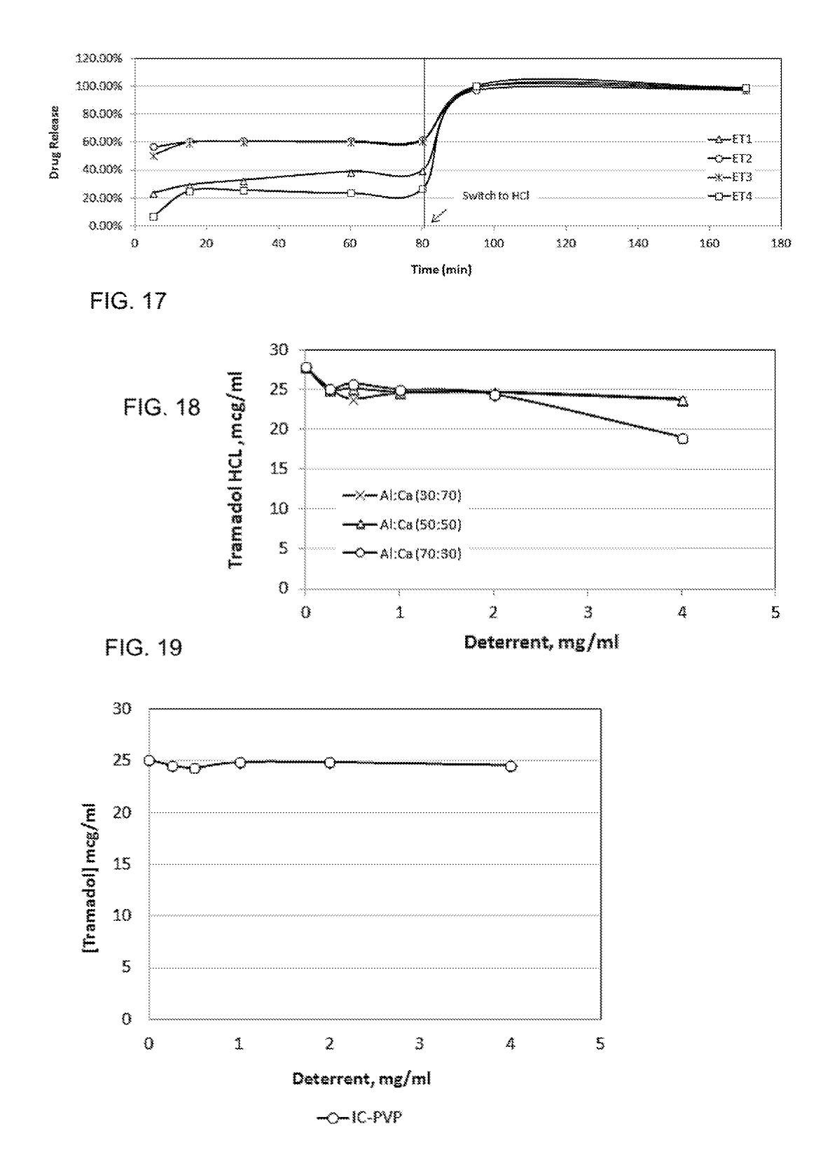

FIG. 17 illustrates that the binding capacity of the IC-SCMC completely disappears in 0.1N HCl solutions.

PC-SCMC Polyacid

A 10 ml volume of 25 μg/ml Tramadol HCl aqueous solution was added to different weights of PC-CMC. Physical crosslinking was achieved using ionic gelation of carboxymethylcellulose in solution sprayed into a solution composed of three different AlCl₃ and CaCl₂ ratios to yield three different physically crosslinked sodium carboxymethylcellulose. Samples were vortexed for 5 sec, and then centrifuged at 1500 rpm for 5 min.

Supernatant was then analyzed for Tramadol concentration using UV-Visible Spectroscopy (UV-1700, Shimadzu).

| Formula | Composition | Aluminum:Calcium in the solution |
|---|---|---|
| A | Al/Ca (SCMC) | 70:30 |
| B | Al/Ca (SCMC) | 50:50 |
| C | Al/Ca (SCMC) | 30:70 |

| Exam- | | Deterrent, | Deterrent, | Abs@ | Tramadol HCl, |
|---|---|---|---|---|---|
| ple | Formula | mg | mg/ml | 271 nm | μg/ml |
| 119 | A | 0 | 0 | 0.1581 | 27.91 |
| 120 | A | 2.5 | 0.25 | 0.1425 | 25.22 |
| 121 | A | 5 | 0.5 | 0.1344 | 23.83 |
| 122 | A | 10 | 1 | 0.1384 | 24.52 |
| 123 | A | 20 | 2 | 0.1394 | 24.69 |
| 124 | A | 40 | 4 | 0.1344 | 23.83 |
| 125 | B | 0 | 0 | 0.1581 | 27.91 |
| 126 | B | 2.5 | 0.25 | 0.1417 | 25.09 |
| 127 | B | 5 | 0.5 | 0.1425 | 25.22 |
| 128 | B | 10 | 1 | 0.1399 | 24.78 |
| 129 | B | 20 | 2 | 0.1395 | 24.71 |
| 130 | B | 40 | 4 | 0.1343 | 23.81 |
| 131 | C | 0 | 0 | 0.1581 | 27.91 |
| 132 | C | 2.5 | 0.25 | 0.1423 | 25.19 |
| 133 | C | 5 | 0.5 | 0.146 | 25.83 |
| 134 | C | 10 | 1 | 0.1417 | 25.09 |
| 135 | C | 20 | 2 | 0.1381 | 24.47 |
| 136 | C | 40 | 4 | 0.1068 | 19.07 |

FIG. 18 illustrates that physically-crosslinked carboxymethyl cellulose does not display deterrence potential, as binding sites are extensively consumed by aluminum and calcium cations.

IC-PVP (a Non-Polyacid)

Effect of Deterrent Concentration

A 10 ml volume of 25 μg/ml Tramadol HCl aqueous solution was added to different weights of IC-PVP. Samples were vortexed for 5 sec, and then centrifuged at 1500 rpm for 5 min. Supernatant was then analyzed for Tramadol concentration using UV-Visible Spectroscopy (UV-1700, Shimadzu).

| Exam- | IC-PVP, | IC-PVP, | Abs@ | Tramadol HCl, |
|---|---|---|---|---|
| ple | mg | mg/ml | 271 nm | μg/ml |
| 137 | 0 | 0 | 0.1416 | 25.07 |
| 138 | 2.5 | 0.25 | 0.1389 | 24.60 |
| 139 | 5 | 0.5 | 0.1375 | 24.36 |
| 140 | 10 | 1 | 0.1404 | 24.86 |
| 141 | 20 | 2 | 0.1404 | 24.86 |
| 142 | 40 | 4 | 0.1388 | 24.59 |

FIG. 19 illustrates that IC-PVP does not display deterrent capacity for Tramadol HCl

Tablet

IC-PVP was formulated into tablets using four different formulas. Tablets were made on a single station carver press at a compression force of approximately 1000 pounds using a 7/16″ punch and die. Tablets were then subjected to dissolution studies using a USP 2 Paddle method (Distek dissolution system 2100A) in 900 ml of ultrapure water at 37.5° C. at a paddle rotational speed of 50 rpm. After 80 minutes, the dissolution medium was changed to 0.1N HCl by adding concentrated hydrochloric acid into the dissolution medium. Tramadol HCl concentration in the dissolution medium was analyzed using UV-visible Spectroscopy (UV-1700, Shimadzu) over time.

| | | Tramadol | | Prosolv | | Actual |
|---|---|---|---|---|---|---|
| | IC-PVP:Tramadol | HCl, | IC-PVP, | SMCC90, | Calculated | weight, |
| Tablet | w/w | mg | mg | mg | weight, mg | mg |
| XT1 | 4:1 | 25 | 100 | 100 | 225 | 223 |
| XT2 | 8:1 | 25 | 200 | 0 | 225 | 216 |
| XT3 | 12:1 | 25 | 300 | 0 | 325 | 319 |
| XT4 | 16:1 | 25 | 400 | 0 | 425 | 417 |

Dissolution Data:

| | | Dissolution | Time, | | Tramadol HCl, | Drug |
|---|---|---|---|---|---|---|
| Example | Tablet | Medium | min | Abs @271 nm | μg/ml | released, % |
| 143 | XT1 | Water | 5 | 0.1563 | 27.60 | 99.37 |
| 144 | XT1 | Water | 15 | 0.1604 | 28.31 | 101.92 |
| 145 | XT1 | Water | 30 | 0.1537 | 27.16 | 97.76 |
| 146 | XT1 | Water | 60 | 0.1583 | 27.95 | 100.61 |
| 147 | XT1 | Water | 80 | 0.1547 | 27.33 | 98.38 |
| 148 | XT1 | 0.1N HCl | 95 | 0.1561 | 26.95 | 97.01 |
| 149 | XT1 | 0.1N HCl | 170 | 0.1566 | 27.04 | 97.33 |
| 150 | XT2 | Water | 5 | 0.1537 | 27.16 | 97.76 |
| 151 | XT2 | Water | 15 | 0.1565 | 27.64 | 99.50 |
| 152 | XT2 | Water | 30 | 0.1538 | 27.17 | 97.82 |
| 153 | XT2 | Water | 60 | 0.155 | 27.38 | 98.57 |
| 154 | XT2 | Water | 80 | 0.1516 | 26.79 | 96.46 |
| 155 | XT2 | 0.1N HCl | 95 | 0.1483 | 25.58 | 92.08 |
| 156 | XT2 | 0.1N HCl | 170 | 0.1497 | 25.82 | 92.97 |
| 157 | XT3 | Water | 5 | 0.1608 | 28.38 | 102.17 |
| 158 | XT3 | Water | 15 | 0.1588 | 28.03 | 100.92 |
| 159 | XT3 | Water | 30 | 0.1555 | 27.47 | 98.88 |
| 160 | XT3 | Water | 60 | 0.1602 | 28.28 | 101.79 |
| 161 | XT3 | Water | 80 | 0.1513 | 26.74 | 96.27 |
| 162 | XT3 | 0.1N HCl | 95 | 0.1559 | 26.91 | 96.88 |
| 163 | XT3 | 0.1N HCl | 170 | 0.1493 | 25.75 | 92.72 |
| 164 | XT4 | Water | 5 | 0.1531 | 27.05 | 97.39 |
| 165 | XT4 | Water | 15 | 0.1536 | 27.14 | 97.70 |
| 166 | XT4 | Water | 30 | 0.1533 | 27.09 | 97.51 |
| 167 | XT4 | Water | 60 | 0.1541 | 27.22 | 98.01 |
| 168 | XT4 | Water | 80 | 0.1511 | 26.71 | 96.14 |
| 169 | XT4 | 0.1N HCl | 95 | 0.1514 | 26.12 | 94.04 |
| 170 | XT4 | 0.1N HCl | 170 | 0.1487 | 125.65 | 92.34 |

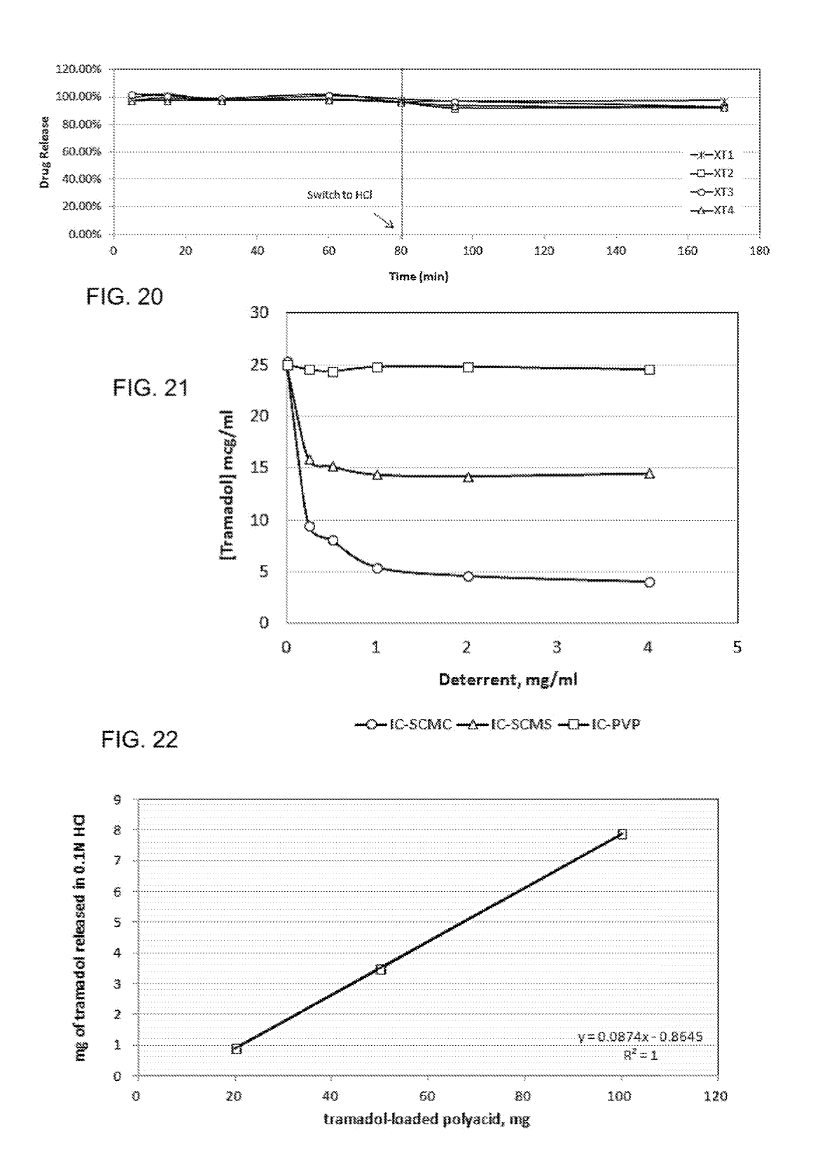

FIG. 20 illustrates that tablets containing different amounts (100-400 mg) of IC-PVP are not abuse-deterrent.

Deterrence Capacity of Different Deterrents

FIG. 21 illustrates relative strength, specifically strong (IC-SCMC), moderate (IC-SCMS), and inactive deterrents.

Drug-Bound IC-SCMC Compositions

With all previous examples (Examples 1-170), drug and the potential deterrent agents were physically mixed or blended for evaluating the deterrence capacity. In this section, drug was first loaded into the deterrent agent particles (in this case IC-SCMC polyacid), and the binding/release capacity of the deterrent agent was then evaluated.

The drug-polyacid complex was prepared by placing 200 mg of IC-SCMC polyacid in a beaker containing 25 ml of a concentrated solution of Tramadol hydrochloride (1000 mg/ml). This slurry was then placed under magnetic stirring for 15 min, after which unbound drug in solution was estimated at 271 nm. The slurry was then transferred into a 50 ml centrifugation tube and ultra-pure water made up to 50 ml. This mixture was then triple washed by being centrifuged at 4000 rpm for 5 minutes and the supernatant discarded and replaced with fresh water each time. After the final rinse, the supernatant was again discarded and the remaining drug complex placed into a glass dish and dried under warm air.

| | | Tramadol HCl | Estimated |
|---|---|---|---|
| | mg of | remaining in | Tramadol HCl |
| mg of | Tramadol HCl | solution after | bound to |
| polyacid | in solution | 15 min, mg | polyacid, mg |
| 200 | 25 | 6.32 | 18.68 |

Three different weight amount of drug-loaded polyacid were placed into 20 ml scintillation vials with ultra-pure water added to 20 ml. The vials were then placed into a water bath at 37.5° C. for 15 min and then centrifuged at 1500 rpm for 5 minutes. Drug concentration in the supernatant was then measured at 271 nm Concentrated hydrochloric acid solution was then added to each vial to produce a 0.1N HCl solution. The vials were again placed into the water bath for 15 min and centrifuged for 5 min. Drug concentration in the supernatant was again measured by UV-visible Spectroscopy (UV-1700, Shimadzu).

| | mg of | [Tramadol | [Tramadol HCl] | |
|---|---|---|---|---|
| | drug- | HCl] | after transition | Tramadol HCl |
| Exam- | loaded | in water, | to 0.1N HCl, | bound to |
| ple | polyacid | mcg/ml | mcg/ml | polyacid, mg |
| 171 | 20 | • | 45.08 | 0.90 |
| 172 | 50 | • | 173.90 | 3.48 |
| 173 | 100 | • | 394.41 | 7.89 |

• Below limit of detection

FIG. 22 illustrates that a tablet containing 300 mg of IC-SCMC bound with Tramadol will be able to release 25 mg Tramadol HCl in 0.1N HCl solution

In another embodiment of the disclosure, the side effects associated with alcohol abuse are decreased by reducing the rate and/or extent of ethanol absorption in the stomach and upper gastrointestinal tract. Alcohol absorption can potentially be reduced by utilizing smart polymers of the disclosure which can preferentially absorb ethanol by their reaction to different gastrointestinal pHs. Alcohol entrapment within the polymer structure greatly reduces its mobility and slows further absorption.

Polymers of this disclosure have a potential to partially absorb ethanol or hydro-alcoholic solutions in the stomach before entering the small intestine. Moreover, smart polymer hydrogels can react to the higher pH change encountered upon exiting the stomach which causes them to expand their structure. As a result, more alcohol or hydro-alcoholic liquids would be entrapped specifically at the site where maximum alcohol absorption occurs within the intestine. Assuming that the implications associated with alcohol abuse are due to the ability of ethanol to be absorbed quickly and to a large extent into the body, this approach will potentially reduce the side effects accompanying alcohol consumption and abuse.

Ethyl alcohol (ethanol, CH₃CH₂OH) is a low molecular weight aliphatic compound, which is completely miscible with water. The hydroxyl (OH) and ethyl (—C2H5) groups of ethyl alcohol are respectively responsible for hydrophilic (water miscibility) and lipophilic (tissue penetration including the brain barrier) properties of this unique chemical.

There are three ways by which alcohol can enter the body: skin, inhalation and of course drinking. Ethyl alcohol taken in via ingestion passes from the mouth down the esophagus and into the stomach, it then moves into the small intestine. At each point along the way, ethyl alcohol can be absorbed into the blood stream. However, the majority of the ethyl alcohol is absorbed from small intestine (approx. 80%), and the stomach (approx. 20%). In general, drinking more alcohol within a certain period of time will result in increased blood alcohol concentrations (BAC) due to more ethyl alcohol being available for absorption into the systemic circulation. However, there are a number of factors that can influence ethyl alcohol absorption from the gastrointestinal tract. These include the rate of gastric emptying, the presence of food, the concentration of the consumed ethyl alcohol, the type of alcoholic beverage consumed, and other factors such as gastrointestinal motility and blood flow.

Knowing alcohol, alcohol absorption and alcohol properties, this disclosure features feasible approaches that can reduce alcohol absorption into the systemic circulation and hence minimize the associated side-effects of abusing alcohol. Polymers of this disclosure are either commercially available or can be tailor-made to trap ethyl alcohol in-vivo, restrict alcohol mobility, and therefore reduce its bioabsorption.

Since ethyl alcohol is primarily absorbed from the upper intestinal GI tract, the ingested alcohol would be either entrapped inside the structure of the polymers of this disclosure, or the mobility of the ingested alcohol would be reduced due to viscosity-enhancing effect of the polymers of this disclosure, or both.

In accordance with the disclosure, the total amounts of alcohol absorbed into the blood circulation will be significantly less if the alcohol is entrapped inside a polymeric structure before being absorbed at its absorption site. The polymer should be able to either selectively absorb ethyl alcohol or to collectively absorb aqueous solutions containing alcohols (hydro-alcoholic solutions). Since alcohol is primarily absorbed in the upper intestine, the polymer should also have higher capacity for absorbing alcohol or hydro-alcoholic solutions at this gastrointestinal segment. Finally the polymer with desirable swelling and absorption properties should be orally administrable. To address this need, the polymer(s) of this disclosure are supplied as particles or granules that can eventually be housed inside a traditional HPMC or gelatin capsule.

A capsule containing such polymer(s) performs as follows: following oral ingestion, the capsule is dissolved in the stomach acid; the polymeric particles are then exposed to the gastric juice containing alcohol, water and HCl; the polymeric particles will start to expand in size by absorbing the gastric juice and alcohol—this process should take place in less than 20 min before the liquid content of the stomach is emptied (half-life of water in stomach is about 25 minutes); the alcohol or the hydro-alcoholic solution will then be physically entrapped into the polymer, no longer directly accessible to the absorption tissue; swollen polymeric particles carrying alcohol or hydro-alcoholic solutions will then pass the pyloric sphincter and move into the upper intestine area where they will be subjected to a higher pH; swollen particles will expand and grow more at higher pH medium of the intestine, so more liquid will be absorbed at the site into the partially swollen particles; swollen particles would eventually and completely be removed from the GI tract. This final stage is somewhat analogous to the elimination of calcium-polycarbophil hydrogel network, which is used to treat constipation, diarrhea and abdominal discomfort.

Polymers with the ability to absorb hydroalcoholic solutions at different pHs may be selected from a group of chemically-crosslinked hydrophilic polymers based on acrylamide, sodium acrylate, potassium acrylate, 2-acrylamido-propane sulfonic acid, potassium sulfopropyl acrylate, acrylic acid, copolymers or terpolymers of these monomers.

The capsule may also contain another group of polymers (alcohol-soluble polymers) that can enhance viscosity of the hydroalcoholic solutions of the stomach and upper intestines. These can be selected from a group of same polymers as mentioned above with linear structure or different class of polymers with great tolerance to alcohol such as polyethylene oxide.

Polymers of the disclosure selected to either absorb hydroalcoholic solutions or to increase their solution viscosity under in-vivo conditions can also be utilized under in-vitro conditions. For example, a tablet composition containing such polymers can absorb the hydroalcoholic solutions that abusers use to extract the drug out of composition. Alternatively, a tablet composition containing such polymers can enhance the viscosity of the hydroalcoholic solutions used by abusers, which would cause the filterability and syringeability of the extraction to become extremely difficult.

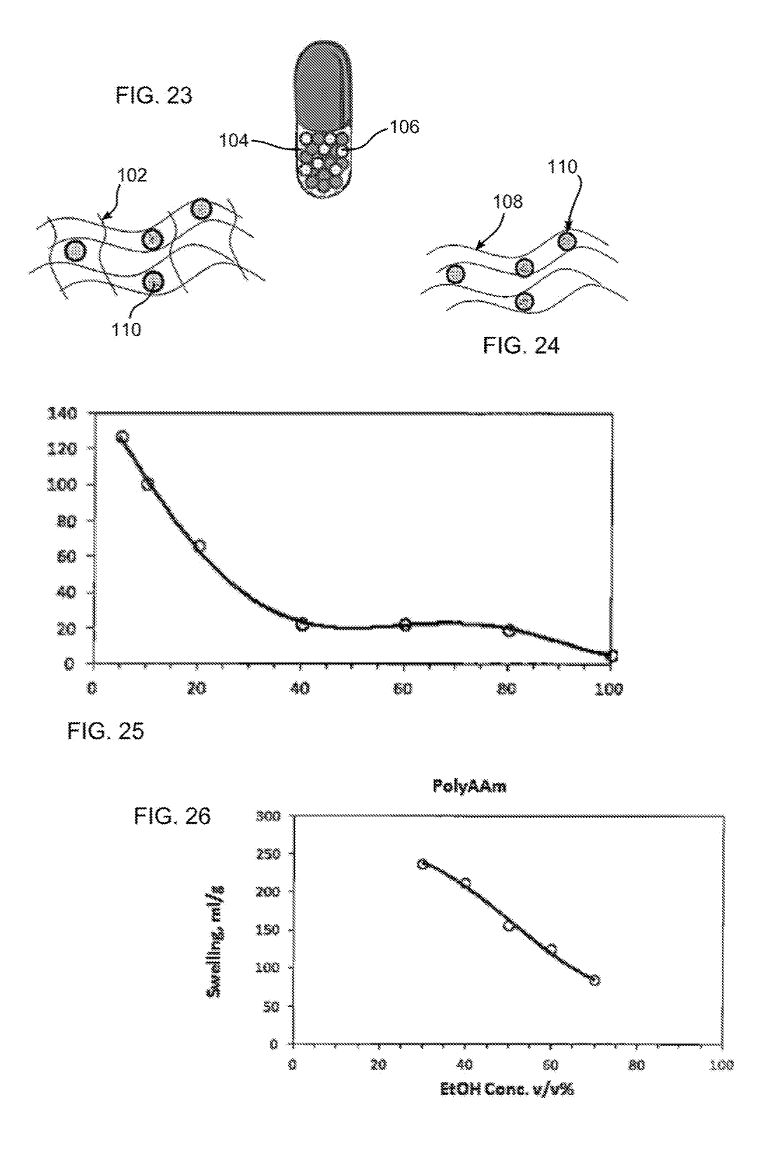

FIGS. 23-24 illustrate entrapment of alcohol molecules 110 within the polymer structure. Crosslinks 102 of polymer chains 108 are diagrammed, as well as alcohol-swellable polymer 104, and alcohol-soluble polymer 106. It should be understood that either or both of polymers 104, 106 may be encapsulated, as illustrated for polymer 106. FIG. 24 illustrates an increasing viscosity of the hydro-alcohol solution.

Viscosity Measurements:

Materials and Methods

Polyethylene oxide (PEO) water-soluble resin (Sentry™Polyox™ WSR Coagulant NF, Dow Chemical, Midland, Mich.), ethyl alcohol 200 Proof USP grade (Pharmco Products Inc, Brookfield, Conn.), Millipore filtered water (=16 Ω*cm). Hydro-alcoholic solutions were prepared using 200 proof ethyl alcohol as 0, 5, 20, 40, 60, 80, 100% v/v alcohol concentration. These solvents were used to make 2% w/v solutions of PEO. The PEO was first passed through a 250 μm mesh screen, and then the powder directly dispersed into the solvents. Solutions were then periodically agitated during the hydration stage, and further stored for a minimum of 24 hours at room temperature prior to use.

Rheological Measurements

Continuous shear rheometry was performed using a Wells-Brookfield cone & plate rheometer (DV-III Ultra, Brookfield Engineering, USA) having a standard cup embedded with a temperature probe and circulating water bath. Measurements were taken with an attached cone of radius 1.2 cm, cone angle of 3°, and at a controlled temperature of 24.96+0.3° C. Test solutions were first centrifuged at 1500 rpm for 5 min to remove entrapped air bubbles, and then a 0.5 ml sample was carefully applied to the middle of the plate and allowed to equilibrate for 2 minutes. Samples where then subjected to increasing shear rates ranging from 2 to 50 sec⁻¹. After 15 seconds of reaching each rate (2, 10, 20, 30, 40, 50 sec⁻¹), a reading was taken to generate the individual rheograms. Due to the suspension nature of the PEO in 100% ethanol, this sample was shaken prior to dispensing onto the plate to evenly disperse the un-dissolved particles.

Swelling Measurements

Materials and Methods

Hydrochloric acid (12N, Fisher Scientific), Ethyl alcohol 200 Proof USP grade (Pharmco Products Inc, Brookfield, Conn.), Millipore filtered water (=16 MΩcm).

Swelling Measured Using Filtration Method

75 mg of the superabsorbent polymer was mixed with 10 mL of hydro-alcoholic solutions at different alcohol concentration. After 2 minutes, the dispersion was filtered and the amount of filtrate (passed through the filter) was measured by volume. The mL of the solution absorbed by the superabsorbent was then obtained by subtracting the filtrate volume out of 10 mL of the original solution.

Swelling Measured Using Bag Method

Five acidic solutions were prepared by using serial dilution of a 0.1N HCl stock solution and water to obtain 0.01 M to 0.01 mM solutions. Five hydroalcoholic solutions were prepared as a 5% w/w solution of ethanol using the various molar acidic solutions previously prepared as the solvent.

The swelling measurements were performed gravimetrically and volumetrically using each SAP in the various acidic and hydroalcoholic solutions. An amount equal to 30 mg of the sample SAP was placed into a commercially obtained basket coffee filter (Fill 'n Brew, Huntingdon Valley, Pa.) that was presoaked with the swelling medium. The loaded filter basket weight was recorded and then placed into a Pyrex glass dish (80×40 mm) filled with 10 ml of the swelling medium and allowed to soak for 120 sec before being removed. Excess solution was allowed to drain for 30 sec and then a second weight measurement recorded. The gram/gram swelling ratio was obtained from the difference in mass of the presoaked and post soaked filter basket minus the weight of the dry polymer over the total SAP dry weight. The remaining liquid in the glass dish was collected and volume recorded. The ml/mg swelling ration was obtained from the difference in swelling medium original volume and that collected over the mg weight of the dry SAP.

Swelling Measured Using Sieve Method

Five acidic solutions were prepared by using serial dilution of a 0.1N HCl stock solution and water to obtain 0.01 M to 0.01 mM solutions. Hydroalcoholic solutions were prepared using 200 proof ethyl alcohol diluted with water to the following concentrations: 0, 5, 20, 40, 60, 80, 100% w/w. Additionally, five hydrochloric-hydroalcoholic solutions were prepared as a 5% w/w solution of ethanol using the various molar acidic solutions previously prepared as the solvent.

The swelling measurements were performed by conventional gravimetric measurement. Each pre-weighed sample (200 mg) was placed into a beaker containing 200 g of the swelling medium under constant stirring (350 rpm) at room temperature for 15 minutes. After this time interval, the solution was placed into a stainless-steel mesh basket (#60) to decant unabsorbed solvent and mildly dried before being weighted on a lab scale to 0.1 g. The gram/gram swelling ratio was obtained as the weight ratio of the swollen to dry superabsorbent.

### Examples

| | | Swelling | | | Swelling |
|---|---|---|---|---|---|
| Example | Superabsorbent | capacity | | Swelling medium | method |
| 1 | Crosslinked polyNaAc¹ | 127 | ml/g | 5% EtOH | Filtration |
| 2 | Crosslinked polyNaAc | 101 | ml/g | 10% EtOH | Filtration |
| 3 | Crosslinked polyNaAc | 66 | ml/g | 20% EtOH | Filtration |
| 4 | Crosslinked polyNaAc | 23 | ml/g | 40% EtOH | Filtration |
| 5 | Crosslinked polyNaAc | 20 | ml/g | 80% EtOH | Filtration |
| 6 | Crosslinked polyNaAc | 5 | ml/g | 100% EtOH | Filtration |
| 7 | Crosslinked polyAAm² | 237 | ml/g | 5% EtOH | Filtration |
| 8 | Crosslinked polyAAm | 212 | ml/g | 10% EtOH | Filtration |
| 9 | Crosslinked polyAAm | 156 | ml/g | 20% EtOH | Filtration |
| 10 | Crosslinked polyAAm | 124 | ml/g | 30% EtOH | Filtration |
| 11 | Crosslinked polyAAm | 84 | ml/g | 40% EtOH | Filtration |
| 12 | Crosslinked AAm-co-NaAc³ | 133 | ml/g | 10% EtOH | Filtration |
| 13 | Crosslinked AAm-co-NaAc | 127 | ml/g | 20% EtOH | Filtration |
| 14 | Crosslinked AAm-co-NaAc | 118 | ml/g | 30% EtOH | Filtration |
| 15 | Crosslinked AAm-co-NaAc | 77 | ml/g | 40% EtOH | Filtration |
| 16 | Crosslinked AAm-co-NaAc | 63 | ml/g | 50% EtOH | Filtration |
| 17 | Crosslinked AAm-co-NaAc | 25 | ml/g | 60% EtOH | Filtration |
| 18 | Crosslinked AAm-co-NaAc | 17 | ml/g | 80% EtOH | Filtration |
| 19 | Crosslinked AAm-co-NaAc | 16 | ml/g | 100% EtOH | Filtration |
| 20 | Crosslinked polySPAK⁴ | 48 | ml/g | Deionized water | Filtration |
| 21 | Crosslinked polySPAK | 47 | ml/g | 10% EtOH | Filtration |
| 22 | Crosslinked polySPAK | 47 | ml/g | 20% EtOH | Filtration |
| 23 | Crosslinked polySPAK | 48 | ml/g | 40% EtOH | Filtration |
| 24 | Crosslinked polySPAK | 44 | ml/g | 60% EtOH | Filtration |
| 25 | Crosslinked polySPAK | 36 | ml/g | 80% EtOH | Filtration |
| 26 | Crosslinked polySPAK | 17 | ml/g | 100% EtOH | Filtration |
| 27 | Crosslinked polyAAm | 235 | g/g | 5% EtOH | Bag |
| 28 | Crosslinked polyAAm | 209 | g/g | 10% EtOH | Bag |
| 29 | Crosslinked polyAAm | 151 | g/g | 20% EtOH | Bag |
| 30 | Crosslinked polyAAm | 79 | g/g | 40% EtOH | Bag |
| 31 | Crosslinked polyAAm | 40 | g/g | pH 1 | Bag |
| 32 | Crosslinked polyAAm | 116 | g/g | pH 2 | Bag |
| 33 | Crosslinked polyAAm | 192 | g/g | pH 3 | Bag |
| 34 | Crosslinked polyAAm | 221 | g/g | pH 4 | Bag |
| 35 | Crosslinked polyAAm | 252 | g/g | pH 5 | Bag |
| 36 | Crosslinked polyAAm | 52 | g/g | pH 1 + 5% EtOH | Bag |
| 37 | Crosslinked polyAAm | 93 | g/g | pH 2 + 5% EtOH | Bag |
| 38 | Crosslinked polyAAm | 187 | g/g | pH 3 + 5% EtOH | Bag |
| 39 | Crosslinked polyAAm | 200 | g/g | pH 4 + 5% EtOH | Bag |
| 40 | Crosslinked polyAAm | 203 | g/g | pH 5 + 5% EtOH | Bag |
| 41 | Crosslinked polyAAm | 282 | g/g | pH 3 | Sieve |
| 42 | Crosslinked polyAAm | 403 | g/g | pH 4 | Sieve |
| 43 | Crosslinked polyAAm | 422 | g/g | pH 5 | Sieve |
| 44 | Crosslinked polyAAm | 280 | g/g | pH 3 + 5% EtOH | Sieve |
| 45 | Crosslinked polyAAm | 384 | g/g | pH 4 + 5% EtOH | Sieve |
| 46 | Crosslinked polyAAm | 391 | g/g | pH 5 + 5% EtOH | Sieve |
| 47 | Crosslinked polyAAm | 416 | g/g | 0% EtOH | Sieve |
| 48 | Crosslinked polyAAm | 387 | g/g | 5% EtOH | Sieve |
| 49 | Crosslinked polyAAm | 371 | g/g | 10% EtOH | Sieve |
| 50 | Crosslinked polyAAm | 288 | g/g | 40% EtOH | Sieve |

| | | | | Shear rate, |
|---|---|---|---|---|
| Example | Superviscosifier, 2 w/v % | Viscosity, cP | Medium | S⁻¹ |
| 51 | Polyethylene oxide | 7937 | 0% EtOH | 2 |
| 52 | Polyethylene oxide | 8731 | 20% EtOH | 2 |
| 53 | Polyethylene oxide | 9525 | 40% EtOH | 2 |
| 54 | Polyethylene oxide | 10318 | 60% EtOH | 2 |
| 55 | Polyethylene oxide | 10318 | 80% EtOH | 2 |
| 56 | Polyethylene oxide | 1587 | 100% EtOH | 2 |
| 57 | Polyethylene oxide | 1746 | 0% EtOH | 20 |
| 58 | Polyethylene oxide | 1825 | 5% EtOH | 20 |
| 59 | Polyethylene oxide | 1825 | 20% EtOH | 20 |
| 60 | Polyethylene oxide | 2063 | 40% EtOH | 20 |
| 61 | Polyethylene oxide | 2143 | 60% EtOH | 20 |
| 62 | Polyethylene oxide | 2143 | 80% EtOH | 20 |
| 63 | Polyethylene oxide | 79 | 100% EtOH | 20 |
| 64 | Polyethylene oxide | 1150 | 0% EtOH | 40 |
| 65 | Polyethylene oxide | 1230 | 5% EtOH | 40 |
| 66 | Polyethylene oxide | 1270 | 20% EtOH | 40 |
| 67 | Polyethylene oxide | 1428 | 40% EtOH | 40 |
| 68 | Polyethylene oxide | 1508 | 60% EtOH | 40 |
| 69 | Polyethylene oxide | 1468 | 80% EtOH | 40 |
| 70 | Polyethylene oxide | 0 | 100% EtOH | 40 |

¹Crosslinked poly(sodium acrylate),
²Crosslinked polyacrylamide,
³Crosslinked sodium acrylate-acrylamide copolymer,
⁴crosslinked poly(sulfopropyl acrylate, potassium)

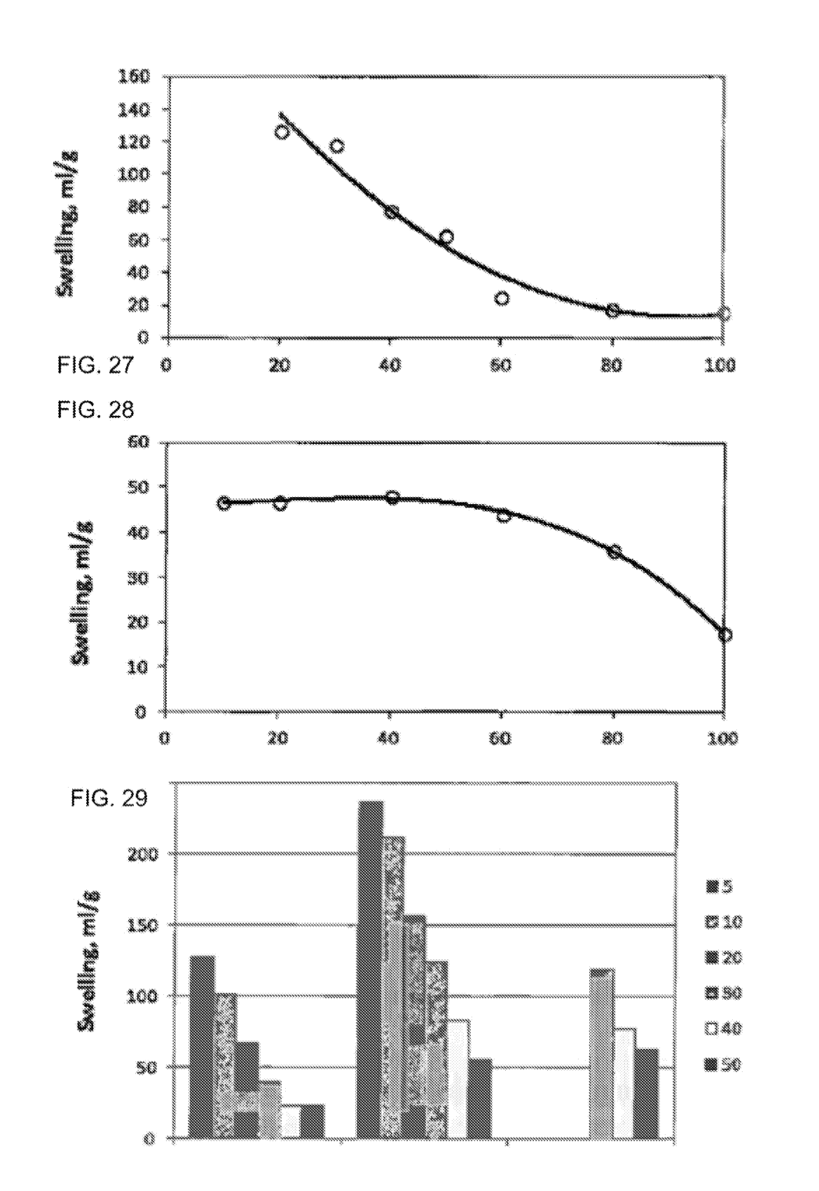

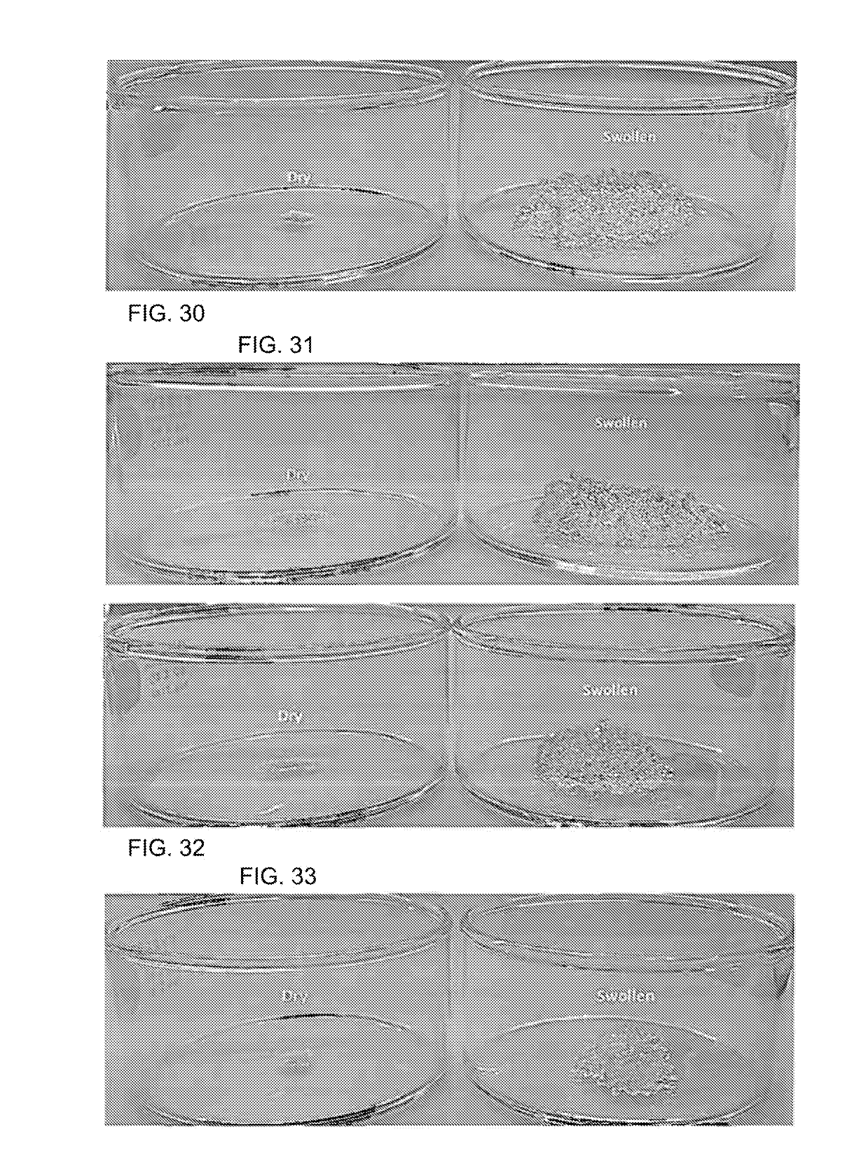

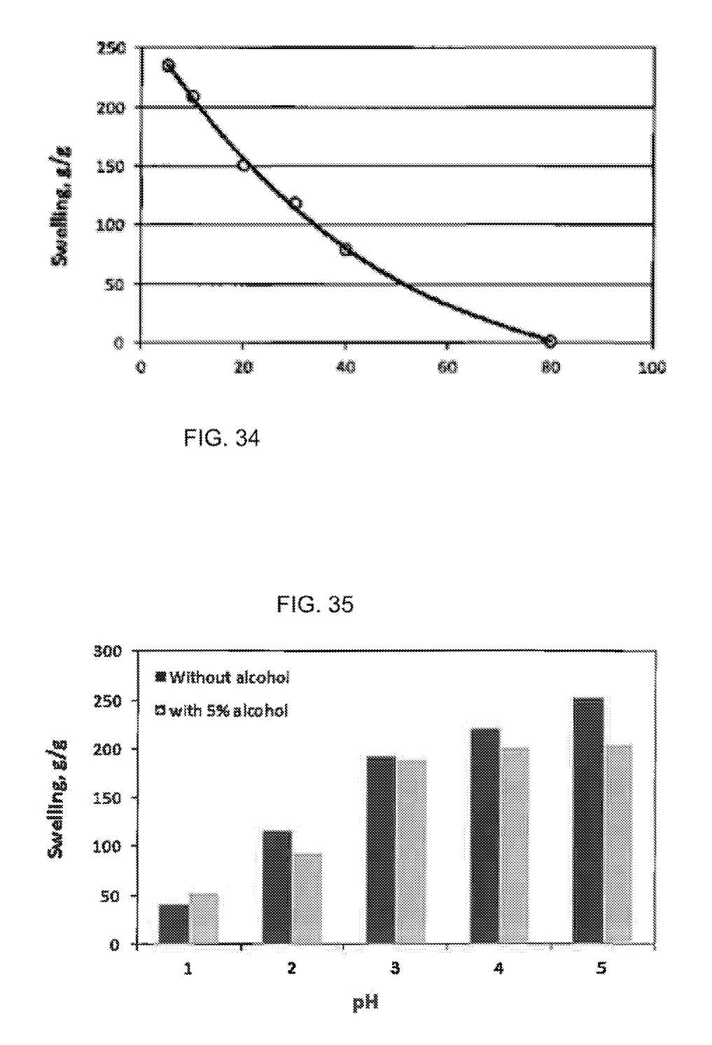

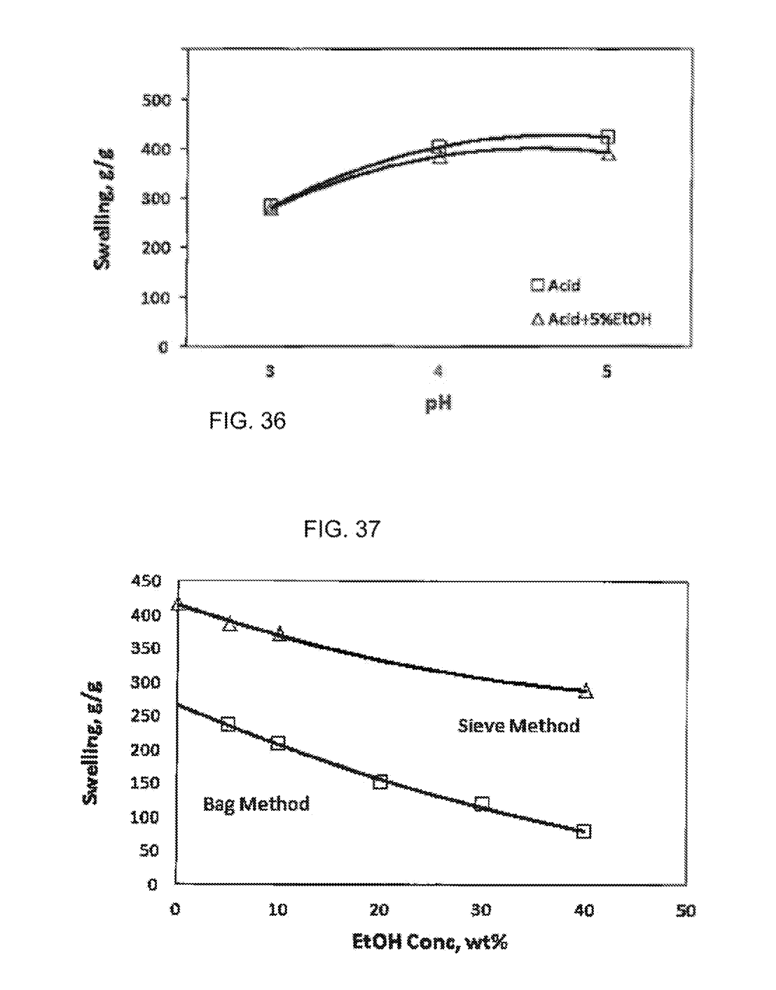

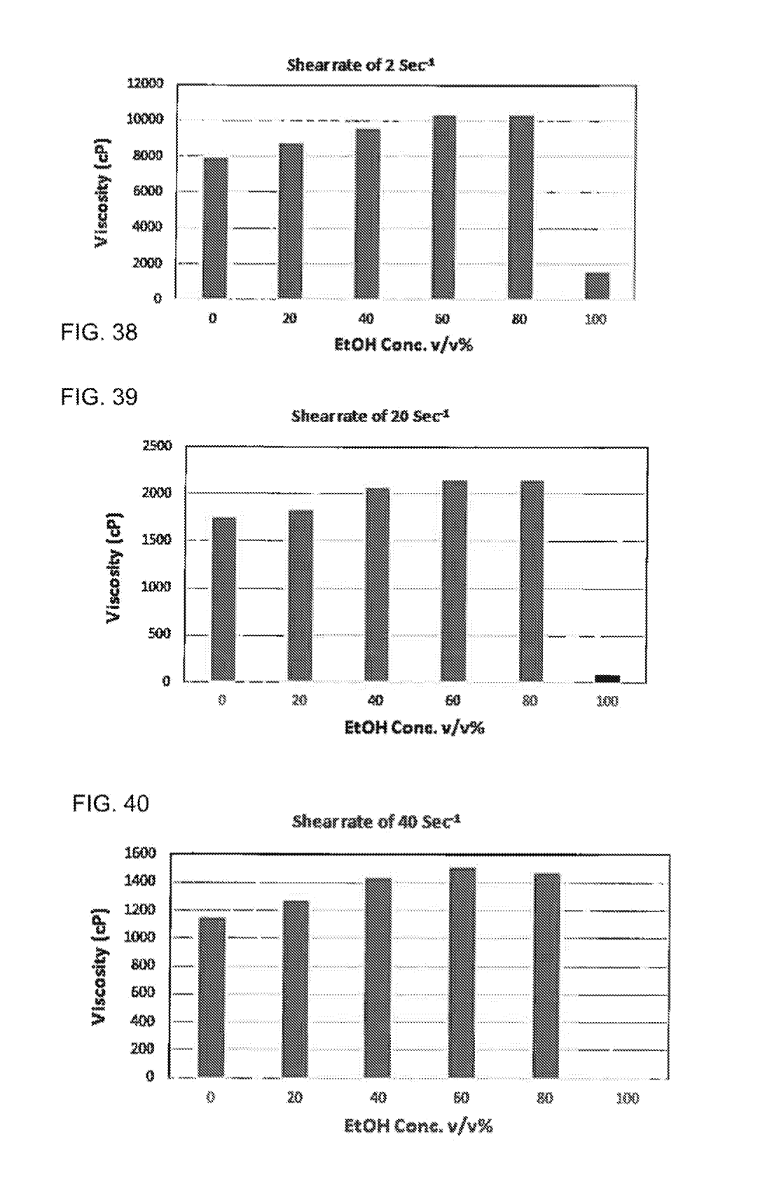

FIG. 25 illustrates volumetric swelling (using filtration method) of crosslinked poly(sodium acrylate) in different alcoholic solutions (Examples 1-6). FIG. 26 illustrates volumetric swelling (using filtration method) of crosslinked polyacrylamide in different alcoholic solutions (Examples 7-11). FIG. 27 illustrates volumetric swelling (using filtration method) of crosslinked copolymer of sodium acrylate and acrylamide in different alcoholic solutions (Examples 12-19). FIG. 28 illustrates volumetric swelling (using filtration method) of crosslinked poly(potassium salt of sulfopropyl acrylate) with superporous structure in different alcoholic solutions (Examples 21-26). FIG. 29 illustrates volume swelling capacity (using filtration method) of crosslinked poly(sodium acrylate), crosslinked polyacrylamide, and crosslinked sodium acrylate and acrylamide copolymer in hydroalcoholic solutions containing 0-50% ethyl alcohol. FIG. 30 illustrates swelling capacity (235 g/g, using bag method) of crosslinked polyacrylamide in 5 wt % EtOH solution (Example 27). FIG. 31 illustrates swelling capacity (209 g/g, using bag method) of crosslinked polyacrylamide in 10 wt % EtOH solution (Example 28). FIG. 32 illustrates swelling capacity (151 g/g, using bag method) of crosslinked polyacrylamide in 20 wt % EtOH solution (Example 29). FIG. 33 illustrates swelling capacity (79 g/g, using bag method) of crosslinked polyacrylamide in 40 wt % EtOH solution (Example 30). FIG. 34 illustrates weight swelling capacity (using bag method) of crosslinked polyacrylamide in different hydroalcoholic solutions at pH of 7 (tests including examples 27-30). FIG. 35 illustrates weight swelling capacity (using bag method) of crosslinked polyacrylamide in different pH medium without (Examples 31-35) and with ethanol (Examples 36-40). FIG. 36 illustrates weight swelling capacity (using sieve method) of crosslinked polyacrylamide in acidic solutions (pH 3-5, Examples 41-43) versus in acidic solutions (pH 3-5) containing 5% ethanol (Examples 44-46). FIG. 37 illustrates weight swelling capacity of crosslinked polyacrylamide in different hydro-alcoholic solutions measured by bag (Examples 27-30) versus sieve methods (47-50). FIG. 38 illustrates cone & plate shear viscosity of 2 wt % solution of Polyox WSR in different alcoholic solutions measured at shear rate of 2 sec⁻¹ and temperature of 22-24° C. (Examples 51-56). FIG. 39 illustrates cone & plate shear viscosity of 2 wt % solution of Polyox WSR in different alcoholic solutions measured at shear rate of 20 sec1 and temperature of 22-24° C. (Examples 57-63). FIG. 40 illustrates cone & plate shear viscosity of 2 wt % solution of Polyox WSR in different alcoholic solutions measured at shear rate of 40 sec1 and temperature of 22-24° C. (Examples 64-70).

In accordance with another embodiment of the disclosure, abusers may swallow a tablet whole with an ingestion of alcohol. The powerful deterrent agents claimed in this disclosure can effectively bind to the drug via their binding sites, and their binding remains stable over a wide variety of abuse conditions as outlined in this disclosure. They can also deter the abuse by insufflation as they are considered to be irritating to nasal passageways when crushed. In order to avoid binding between the active ingredient and the deterrent agent under regular administration by non-abusing patients, the deterrent agent is coated with certain polymers which protect the drug from interacting with the deterrent agent in solution.

Crushing

In accordance with the disclosure, one or more clays are mixed with an aqueous solution of the drug (e.g., Tramadol HCl), and the mixture is vacuum-dried at low temperature. The dried drug-clay complex will then be used in the preparation of tablet. Since the drug is not free and already bound to the structure of the clay, it will not be easily released if the abusers sniff the crushed tablet. Moreover, the clay particles are irritating if crushed into fine particles.

Abuse

The tablet will contain an ionic drug (e.g., Tramadol HCl), clay (deterrent agent), and other necessary excipients required to prepare the tablet dosage form. Once in solution, the clay will immediately form a strong complex with the basic drug, and prevents the abusable drug from being extracted into solution. In order for the drug to be released under regular method of administration by regular patent, one embodiment of this disclosure discloses coated clay particles and aggregates which only function if the clay particles are tampered.

Alcohol Co-Ingestion

The clay-drug complex of this disclosure will resist highly concentrated hydroalcoholic solutions over an applicable range of alcohol concentrations commonly used in the abuse process.

The agent bentonite (advantageously calcium bentonite) can be used as the clay component of all preparations and pharmaceutical compositions in the examples herein, although other clay component can be used, as would be understood by one skilled in the art.

### Examples

### Clay Binding of Tramadol Hydrochloride in Aqueous Solutions

Effect of Clay Concentration in Solution

A 10 ml of 25 μg/ml Tramadol HCl aqueous solution was added to different weights of clay. Dispersions were vortexed for 5 sec and then centrifuged at 1500 rpm for 5 min. Supernatant was then analyzed for Tramadol concentration using UV-Visible Spectroscopy at 271 nm (UV-1700, Shimadzu).

| | | | | | | [Tramadol | |
|---|---|---|---|---|---|---|---|
| | | | | | [Tramadol | HCl] in | % of |
| | | | | | HCl] in | solution | total |
| | | | | | beginning | after | drug |
| | Clay, | Clay, | Clay/Tramadol, | Abs @ | solution, | addition of | bound |
| Example | mg | mg/ml | mg/mg | 271 nm | μg/ml | Clay, μg/ml | to Clay |
| 1 | 0 | 0 | 0:1 | 0.1510 | 24.35 | 24.35 | 0.00 |
| 2 | 0.5 | 0.05 | 2:1 | 0.1019 | 24.35 | 16.17 | 33.61 |
| 3 | 1 | 0.1 | 4:1 | 0.1011 | 24.35 | 16.03 | 34.15 |
| 4 | 2.5 | 0.25 | 10:1 | 0.0718 | 24.35 | 11.15 | 54.21 |
| 5 | 5 | 0.5 | 20:1 | 0.0380 | 24.35 | 5.52 | 77.34 |
| 6 | 10 | 1 | 40:1 | 0.0128 | 24.35 | 1.32 | 94.59 |
| 7 | 20 | 2 | 80:1 | 0.0110 | 24.35 | 1.02 | 95.82 |
| 8 | 40 | 4 | 160:1 | 0.0028 | 24.35 | −0.35 | 101.44 |

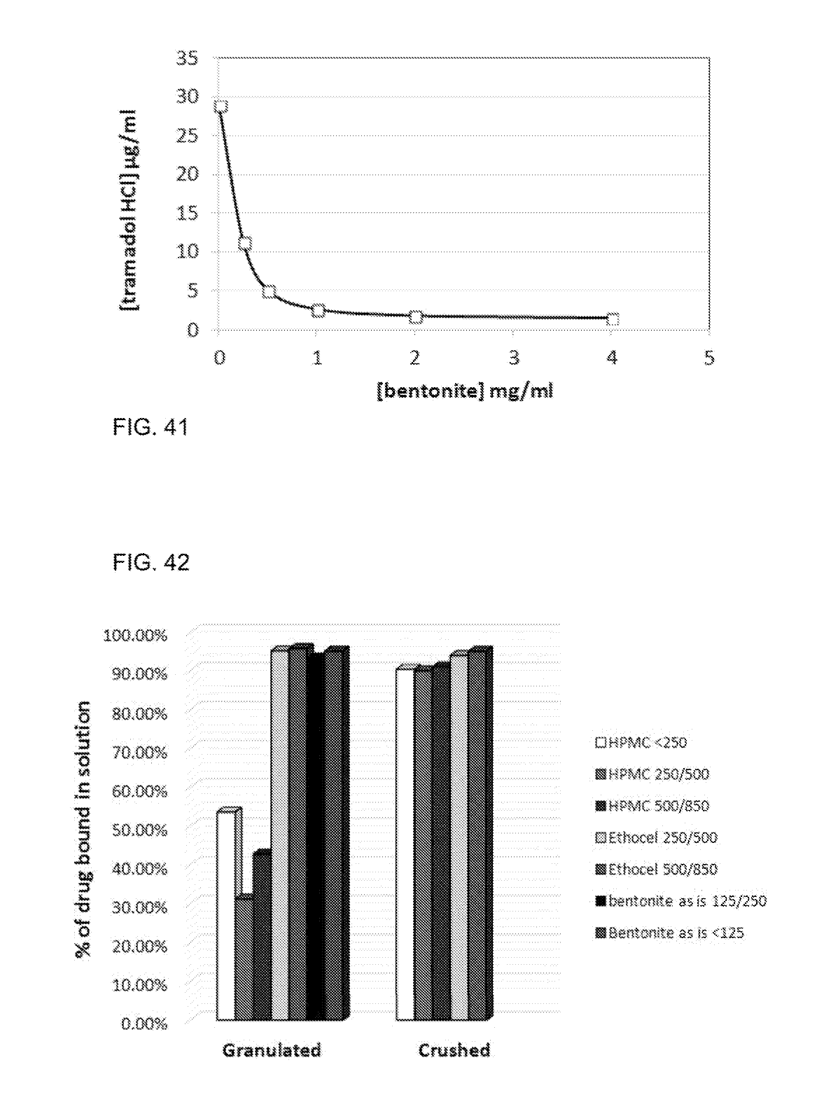

FIG. 41 illustrates that Tramadol HCl can effectively be captured by the bentonite clay. The effect will be leveled off at higher clay concentrations.

Effect of Clay Particle Size

Clay powder as supplied was screened to obtain two particle size ranges (<125 μm and 125-250 μm). A 10 ml volume of 25 μg/ml Tramadol HCl aqueous solution was then added to 20 mg of clay. Samples were vortexed for 5 sec, and then centrifuged at 1500 rpm for 5 min. Supernatant was then analyzed for Tramadol concentration using UV-Visible Spectroscopy at 271 nm (UV-1700, Shimadzu).

| | | Clay | Clay/ | | [Tramadol | |
|---|---|---|---|---|---|---|
| | Clay, | particle | Tramadol, | Abs @ | HCl], | % of drug |
| Example | mg | size, μm | mg/mg | 271 nm | μg/ml | bound |
| 9 | 0 | — | 0:1 | 0.1395 | 25.07 | 0 |
| 10 | 20 | <125 | 80:1 | 0.0083 | 1.22 | 95.13 |
| 11 | 20 | 125-250 | 80:1 | 0.0107 | 1.65 | 93.38 |

The foregoing data shows insignificant difference in binding capacity between clay particles at <125 μm and 125-250 μm sizes, although finer particles display a better binding capacity.

Effect of Clay Granulation

Clay granules were made by first wetting dry clay powder with either a 7% w/w hypromellose solution in water or a 1% w/w ethyl cellulose solution in ethanol. The wet mass produced was then passed through a #6 sieve, and the resultant granules dried out under hot air at 68° C. Dried granules were then screened for particle size ranges. A 10 ml volume of 25 μg/ml Tramadol HCl aqueous solution was then added to 20 mg of granules from each size range. Dispersions were then vortexed for 5 sec, and centrifuged at 1500 rpm for 5 min. Supernatant was then analyzed for Tramadol concentration using UV-Visible Spectroscopy at 271 nm (UV-1700, Shimadzu). Additionally, the effect of Tramadol binding, when the granules were reduced in particle size (crushed), was also measured. Granules were crushed using a glass mortar and pestle with 40 mg of sample triturated 50 times in a clock-wise direction.

| | | Clay | Clay | | | [Tramadol | |
|---|---|---|---|---|---|---|---|
| | Clay, | particle | granulating | Clay/Tramadol, | Abs @ | HCl], | % of drug |
| Example | mg | size, μm | solution | mg/mg | 271 nm | μg/ml | bound |
| 12 | 0 | — | | 0:1 | 0.1395 | 25.07 | 0 |
| 13 | 20 | <250 | HPMC | 80:1 | 0.0653 | 11.58 | 53.67 |
| 14 | 20 (crushed) | <250 | HPMC | 80:1 | 0.0148 | 2.40 | 90.40 |
| 15 | 20 | 250-500 | HPMC | 80:1 | 0.0961 | 17.18 | 31.27 |
| 16 | 20 (crushed) | 250-500 | HPMC | 80:1 | 0.0153 | 2.49 | 90.04 |
| 17 | 20 | 500-850 | HPMC | 80:1 | 0.0802 | 14.29 | 42.84 |
| 18 | 20 (crushed) | 500-850 | HPMC | 80:1 | 0.0139 | 2.24 | 91.05 |
| 19 | 20 | 250-500 | EC | 80:1 | 0.0082 | 1.20 | 95.20 |
| 20 | 20 (crushed) | 250-500 | EC | 80:1 | 0.0099 | 1.51 | 93.96 |
| 21 | 20 | 500-850 | EC | 80:1 | 0.0074 | 1.05 | 95.78 |
| 22 | 20 (crushed) | 500-850 | EC | 80:1 | 0.0084 | 1.24 | 95.05 |

FIG. 42 illustrates that HPMC can effectively reduce the binding effect of the clay granulated particles. Once crushed, entrapped clay particles can bind to the drug very effectively.

Clay Binding of Tramadol Hydrochloride in Tablets:

Effect of Clay Concentration

Clay was formulated into tablets using four different formulas. Tablets were made on a single station Carver press at a compression force of approximately 1000 pounds using a 7/16′ punch and die. Tablets were then subjected to dissolution studies using a USP 2 Paddle method (Distek dissolution system 2100A) in 900 ml of ultrapure water at 37.5° C. at a paddle rotational speed of 50 rpm. After 80 minutes, the dissolution medium was changed to 0.1N HCl by the addition of concentrated hydrochloric acid into the dissolution medium. Tramadol HCl concentration in the dissolution medium was analyzed using UV-Visible Spectroscopy at 271 nm (UV-1700, Shimadzu) over time.

Tablet Compositions

| | Clay/ | Tramadol | | Prosolv | Calculated | Actual |
|---|---|---|---|---|---|---|
| | Tramadol | HCl, | Clay, | SMCC 90, | weight, | weight, |
| Formula | wt/wt | mg | mg | mg | mg | mg |
| B1 | 4:1 | 25 | 100 | 100 | 225 | 217.2 |
| B2 | 8:1 | 25 | 200 | 0 | 225 | 221.9 |
| B3 | 12:1 | 25 | 300 | 0 | 325 | 321.8 |
| B4 | 16:1 | 25 | 400 | 0 | 425 | 424.4 |

Dissolution Data

| | | | | | [Tramadol | % |
|---|---|---|---|---|---|---|
| | | Dissolution | Time, | Abs @ | HCl], | of drug |
| Example | Formula | Medium | min | 271 nm | μg/ml | released |
| 23 | B1 | Water | 5 | 0.0449 | 7.87 | 28.34 |
| 24 | B1 | Water | 15 | 0.056 | 9.89 | 35.61 |
| 25 | B1 | Water | 30 | 0.0509 | 8.96 | 32.27 |
| 26 | B1 | Water | 60 | 0.0527 | 9.29 | 33.45 |
| 27 | B1 | Water | 80 | 0.0497 | 8.75 | 31.48 |
| 28 | B1 | 0.1N HCl | 95 | 0.0725 | 12.95 | 46.60 |
| 29 | B1 | 0.1N HCl | 170 | 0.0768 | 13.73 | 49.42 |
| 30 | B2 | Water | 5 | 0.025 | 4.25 | 15.32 |
| 31 | B2 | Water | 15 | 0.0265 | 4.53 | 16.30 |
| 32 | B2 | Water | 30 | 0.0289 | 4.96 | 17.87 |
| 33 | B2 | Water | 60 | 0.03 | 5.16 | 18.59 |
| 34 | B2 | Water | 80 | 0.0316 | 5.45 | 19.64 |
| 35 | B2 | 0.1N HCl | 95 | 0.037 | 6.49 | 23.37 |
| 36 | B2 | 0.1N HCl | 170 | 0.0437 | 7.71 | 27.75 |
| 37 | B3 | Water | 5 | 0.0153 | 2.49 | 8.97 |
| 38 | B3 | Water | 15 | 0.0153 | 2.49 | 8.97 |
| 39 | B3 | Water | 30 | 0.0208 | 3.49 | 12.57 |
| 40 | B3 | Water | 60 | 0.0177 | 2.93 | 10.54 |
| 41 | B3 | Water | 80 | 0.0186 | 3.09 | 11.13 |
| 42 | B3 | 0.1N HCl | 95 | 0.0327 | 5.71 | 20.55 |
| 43 | B3 | 0.1N HCl | 170 | 0.0438 | 7.73 | 27.82 |
| 44 | B4 | Water | 5 | 0.0114 | 1.78 | 6.41 |
| 45 | B4 | Water | 15 | 0.0125 | 1.98 | 7.13 |
| 46 | B4 | Water | 30 | 0.0118 | 1.85 | 6.68 |
| 47 | B4 | Water | 60 | 0.0137 | 2.20 | 7.92 |
| 48 | B4 | Water | 80 | 0.0125 | 1.98 | 7.13 |
| 49 | B4 | 0.1N HCl | 95 | 0.0242 | 4.16 | 14.99 |
| 50 | B4 | 0.1N HCl | 170 | 0.0323 | 5.64 | 20.29 |

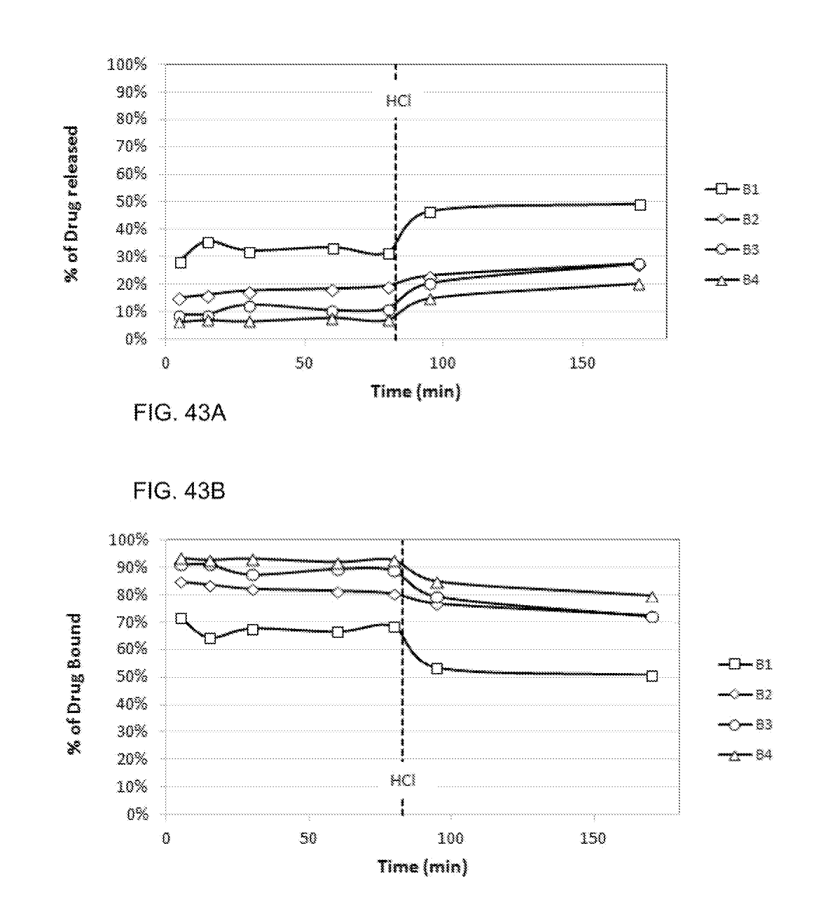

FIGS. 43A and 43B illustrate that clay is more effective at higher concentration in the tablet. However, a drug-clay complex prepared at different drug clay ratios will remain quite stable in water, but become partially unstable in 0.1N HCl solution.

Clay Coated Particles

Effect of Enteric Coating

Clay granules were made by mixing 3 g of clay powder with 8 g of a 2 w/w % hydroxypropyl methylcellulose (K100M premium) solution and 5 g of a 2.5% w/w copovidone (Kollidon VA 64) to create a wet mass that was passed through a #60 sieve, and resultant particles dried out at 68° C. Particles were then coated by spray nozzle using a clear film coating of the following composition.

| Ingredient | Composition, wt % |
|---|---|
| Kollicoat MAE 100P | 21 |
| Polyethylene glycol | 4.2 |
| Water | 74.8 |
| TOTAL | 100 |

After coating, the granules were either used as is or crushed using a glass mortar and pestle (triturated in a clock-wise direction for 25 revolutions). Then a 10 ml of 25 μg/ml Tramadol HCl solution in water or 0.1N HCl was added to 20 mg of clay samples. Each mixture was then vortexed for 5 sec, and centrifuged at 1500 rpm for 5 min. Supernatant was then analyzed for Tramadol concentration using UV-Visible Spectroscopy at 271 nm (UV-1700, Shimadzu).

| | | | Abs @ | Abs @ | | [Tramadol | |
|---|---|---|---|---|---|---|---|
| | | | 271 nm | 271 nm | [Tramadol | HCl] in | % of |
| | | Tramadol 25 μg/ml | Tramadol | after | HCl] in | solution | total |
| | Clay | stock | stock | addition of | stock | after Clay | drug |
| | Particles, | solution | solution, | Clay | solution, | addition, | bound to |
| Example | mg | composition | 25 μg/ml | particles | μg/ml | μg/ml | Clay |
| 51 | 20 | water | 0.1486 | 0.1123 | 26.72 | 20.13 | 24.68 |
| 52 | 20 | water | 0.1486 | 0.0372 | 26.72 | 6.47 | 75.78 |
| | (crushed) | | | | | | |
| 53 | 20 | 0.1N HCl | 0.1541 | 0.1532 | 27.78 | 27.62 | 0.59 |
| 54 | 20 | 0.1N HCl | 0.1541 | 0.0938 | 27.78 | 16.82 | 39.46 |
| | (crushed) | | | | | | |

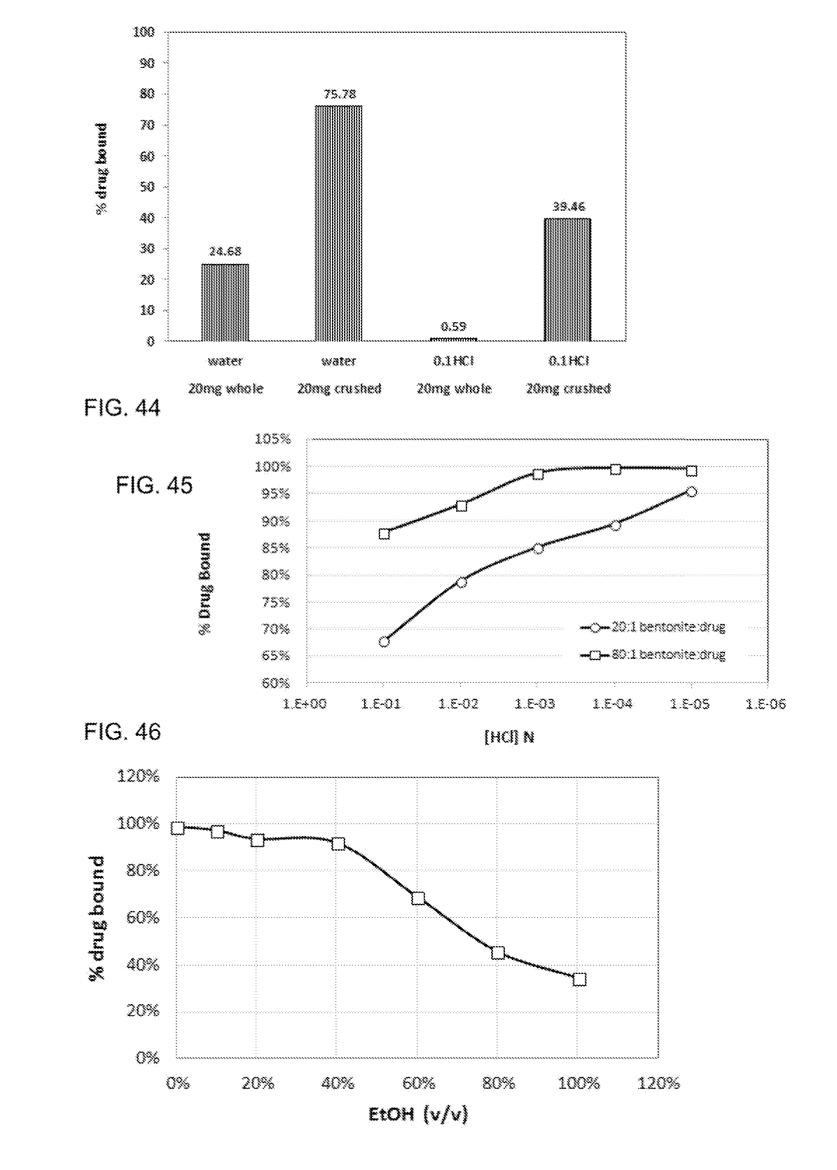

FIG. 44 illustrates the effect of enteric coating on binding capacity of the clay particles

Clay Abuse Studies

Effect of pH

A 10 ml of 25 μg/ml Tramadol HCl aqueous solution made of different molar concentrations of HCl was added to two different weights of clay. Dispersions were vortexed for 5 sec, and then centrifuged at 1500 rpm for 5 min. Supernatant was then analyzed for Tramadol concentration using UV-Visible Spectroscopy at 271 nm (UV-1700, Shimadzu).

| | | | | | | [Tramadol | |
|---|---|---|---|---|---|---|---|
| | | | Abs @ | Abs @ | [Tramadol | HCl] in | |
| | | | 271 nm of | 271 nm | HCl] in | solution | % of total |
| | | | Tramadol | after | starting | after Clay | Tramadol |
| | Clay, | Normality | starting | addition | solution, | addition, | bound to |
| Example | mg | of solution | solution | of Clay | μg/ml | μg/ml | Clay |
| 55 | 5 | 0.1 | 0.1394 | 0.0458 | 25.11 | 8.09 | 67.78 |
| 56 | 20 | 0.1 | 0.1394 | 0.0182 | 25.11 | 3.07 | 87.76 |
| 57 | 5 | 0.01 | 0.1532 | 0.0363 | 24.72 | 5.23 | 78.83 |
| 58 | 20 | 0.01 | 0.1532 | 0.0154 | 24.72 | 1.75 | 92.92 |
| 59 | 5 | 0.001 | 0.1489 | 0.0264 | 24.00 | 3.58 | 85.07 |
| 60 | 20 | 0.001 | 0.1489 | 0.0068 | 24.00 | 0.32 | 98.68 |
| 61 | 5 | 0.0001 | 0.1506 | 0.0204 | 24.28 | 2.58 | 89.36 |
| 62 | 20 | 0.0001 | 0.1506 | 0.0055 | 24.28 | 0.10 | 99.59 |
| 63 | 5 | 0.00001 | 0.1331 | 0.0106 | 21.37 | 0.95 | 95.55 |
| 64 | 20 | 0.00001 | 0.1331 | 0.0056 | 21.37 | 0.12 | 99.45 |

FIG. 45 illustrates the stability of the clay-drug complex at different pHs, especially at low pHs. At pH 1, there is still 65-85% of the drug bound to the clay particles.

Effect of Ions

A 10 ml of 25 μg/ml Tramadol HCl in normal saline (0.9% NaCl) was added to two different weights of clay. Dispersions were vortexed for 5 sec, and then centrifuged at 1500 rpm for 5 min. Supernatant was then analyzed for Tramadol concentration using UV-Visible Spectroscopy at 271 nm (UV-1700, Shimadzu).

| | | | | | | [Tramadol | |
|---|---|---|---|---|---|---|---|
| | | | Abs @ | Abs @ | [Tramadol | HCl] in | |
| | | | 271 nm of | 271 nm | HCl] in | solution | % of total |
| | | | Tramadol | after | starting | after Clay | Tramadol |
| | Clay, | NaCl, | starting | addition of | solution, | addition, | bound to |
| Example | mg | w/v % | solution | Clay | μg/ml | μg/ml | Clay |
| 65 | 5 | 0.9 | 0.0488 | 0.0216 | 25.81 | 12.86 | 50.18 |
| 66 | 20 | 0.9 | 0.0488 | 0.01235 | 25.81 | 8.45 | 67.25 |

Effect of Hydroalcoholic Solutions

A 10 ml of 25 μg/ml Tramadol HCl in various hydroalcoholic concentrations was added to 20 mg of clay. Dispersions were vortexed for 5 sec, and then centrifuged at 1500 rpm for 5 min. Supernatant was then analyzed for Tramadol concentration using UV-Visible Spectroscopy at 271 nm (UV-1700, Shimadzu).

| | | | | Abs @ | | [Tramadol | |
|---|---|---|---|---|---|---|---|
| | | | Abs @ | 271 nm, | [Tramadol | HCl] in | |
| | | | 271 nm of | after | HCl] in | solution | % of total |
| | | | Tramadol | addition of | starting | after Clay | Tramadol |
| | Clay, | EtOH, | starting | 20 mg | solution, | addition, | bound to |
| Example | mg | w/w % | solution | Clay | μg/ml | μg/ml | Clay |
| 67 | 20 | 0 | 0.1559 | 0.0067 | 25.17 | 0.30 | 98.81 |
| 68 | 20 | 10 | 0.1472 | 0.0088 | 23.72 | 0.65 | 97.26 |
| 69 | 20 | 20 | 0.1439 | 0.0139 | 23.17 | 1.50 | 93.53 |
| 70 | 20 | 40 | 0.1465 | 0.0162 | 23.60 | 1.88 | 92.02 |
| 71 | 20 | 60 | 0.1436 | 0.0479 | 23.12 | 7.17 | 69.00 |
| 72 | 20 | 80 | 0.151 | 0.0845 | 24.35 | 13.27 | 45.52 |
| 73 | 20 | 100 | 0.1682 | 0.1091 | 25.38 | 16.69 | 34.24 |

FIG. 46 illustrates that stability of drug clay complex in different hydroalcoholic solutions. Data shows complex will remain stable in water-alcohol solutions up to 40% alcohol, and then gradually loses its stability at higher alcohol concentrations. About 35% of the drug still remains bound to the clay particles in 100% alcohol.

Tramadol-Clay Complex

Preparation of Tramadol-Clay Complex

A drug complex was prepared by placing 600 mg of sieved clay (particle size range 45-125 μm) into glass scintillation vial containing 20 ml of a concentrated solution of Tramadol hydrochloride (1000 μg/ml). The dispersion was vortexed for one minute, and then allowed to settle at room temperature for 15 min, after which unbound drug in solution was estimated using UV-Visible Spectroscopy at 271 nm (UV-1700, Shimadzu). The dispersion was then centrifuged for 5 minutes at 1500 rpm, and the supernatant discarded and replaced with fresh ultrapure water. The washing and centrifugation steps were conducted an additional three times to remove any unbound Tramadol. After the final rinse, the supernatant was again discarded and the remaining drug complex placed into a glass dish and dried out under warm air at 68° C.

| | Tramadol | Tramadol HCl | Estimated Tramadol |
|---|---|---|---|
| Bentonite, | HCl in | remaining in | HCl bound to |
| mg | solution, mg | solution after 15 min, mg | Bentonite, mg |
| 600 | 20 | 0.14 | 19.86 |

Evaluation of Tramadol-Clay Complex

A mass of 25 mg of the drug-clay complex was placed into separate glass scintillation vials. To each vial was then added 10 ml of either water, 0.1N HCl, 0.9% w/v sodium chloride, or 200 proof ethanol (100% v/v). Each vial was then vortexed for 5 seconds and centrifuged at 1500 rpm for 5 minutes. Drug concentration in the supernatant was then measured by UV-Visible Spectroscopy (UV-1700, Shimadzu) at 271 nm.

| | Tramadol- | | Theoretical | Tramadol | Tramadol |
|---|---|---|---|---|---|
| | clay | Disso- | Tramadol | HCl | HCl |
| | complex, | lution | content in | in solution, | released, |
| Example | mg | Medium | complex, μg | μg/ml | μg |
| 74 | 20 | Water | 827.65 | 0.44 | 4.43 |
| 75 | 20 | 0.1N | 827.65 | 4.42 | 44.18 |
| | | HCl | | | |
| 76 | 20 | Ethanol | 827.65 | 5.33 | 53.28 |
| 77 | 20 | 0.9% | 827.65 | 18.30 | 183.02 |
| | | NaCl | | | |

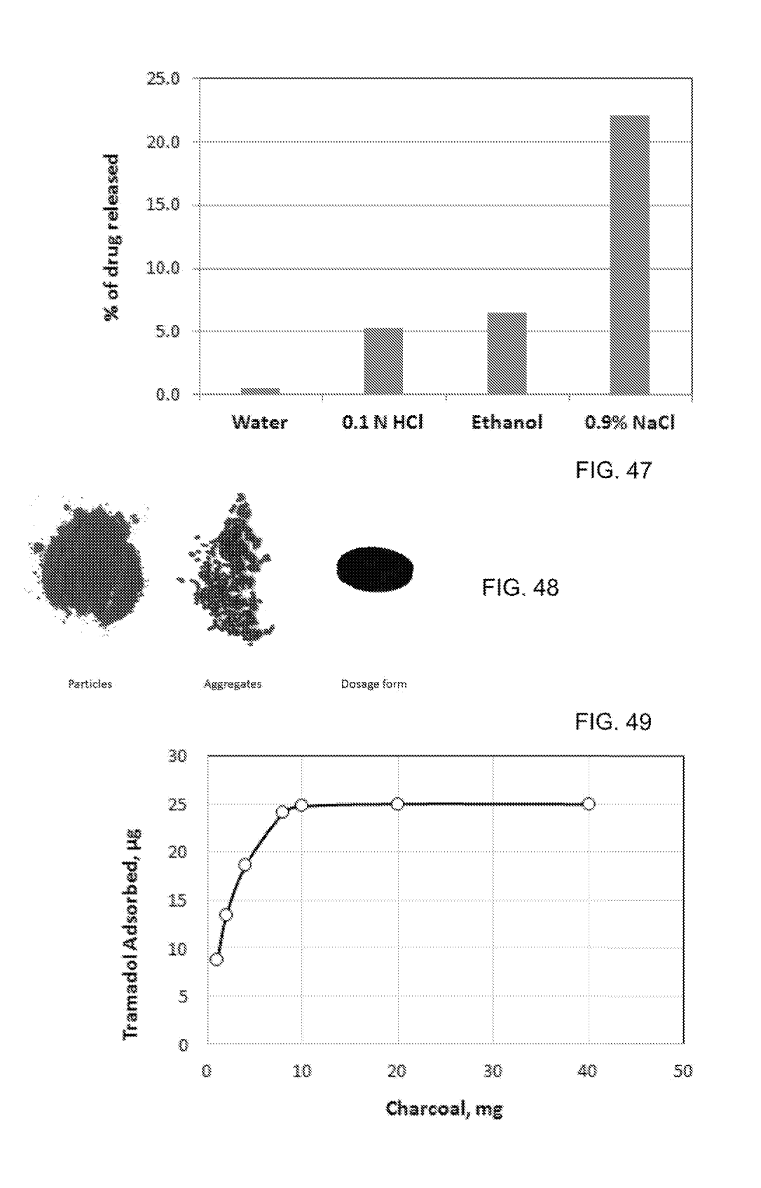

FIG. 47 illustrates the amount of Tramadol released from the drug-clay complex in different extraction or dissolution medium

In another embodiment, a super-deterrent agent of this disclosure can effectively adsorb the drug into its adsorption sites, where the drug cannot be displaced or extracted under wide variety of abuse conditions as outlined in this disclosure.

Due to its super-adsorption property and aversiveness (blackness and irritation), we used an activated charcoal or medicinal carbon (Charcoal Activated Powder USP, HUMCO, Texarkana, Tex.) to represent an aversive super-deterrent agent in pharmaceutical preparations of the disclosure, and to evaluate a function of medicinal carbon as a aversive super-deterrent agent in compositions containing abusable medications, such as Tramadol HCl.

Crushing

This super-deterrent agent can effectively adsorb the drug into its adsorption sites, where the drug cannot be displaced or extracted under wide variety of abuse conditions as outlined in this disclosure. The super-deterrent agent of this disclosure can also deter the abuse by insufflation due to its pitched-black color, and due to the substantial coverage area that its particles provide. In order to avoid adsorption of the drug into super-deterrent particles under regular administration by non-abusing patients, the particles or aggregates of the super-deterrent agent are coated with certain polymers which protect the drug from interacting with the deterrent agent in solution.

Activated charcoal is used to deter abuse by crushing in three ways. First, it can adsorb the drug in the wet nasal passageways, which slows down the drug absorption and causes its reduced bioavailability. Second, the charcoal particles are pitch-black with great coverage area, which can avert the abuse as an aversive agent. Lastly, according to the MSDS of the medicinal product, charcoal may cause respiratory tract irritation.

Abuse

A tablet of this embodiment can contain an ionic drug (e.g., Tramadol HCl), an activated charcoal (super-deterrent agent), and other necessary excipients required to prepare the tablet dosage form. Once in solution, the activated charcoal will immediately adsorb the basic drug, and prevent the abusable drug from being extracted into solution. In order for the drug to be released under regular method of administration by regular patient, coated activated charcoal particles and aggregates of this embodiment only function if the charcoal particles are subjected to abuse.

Alcohol Co-Ingestion

In another embodiment, the drug-adsorbed charcoal particles or aggregates of this disclosure will resist moderate hydroalcoholic solutions over an applicable range of alcohol concentrations commonly used in the abuse process.

FIG. 48 illustrates particles, aggregates and dosage of activated charcoal as disclosed herein.

### Examples

### Charcoal Adsorption of Tramadol HCl in Solution

Effect of Charcoal Concentration

A 10 ml of 25 μg/ml Tramadol HCl aqueous solution was added to different weight amounts of charcoal powder. Dispersions were vortexed for 5 sec, and then centrifuged at 1500 rpm for 5 min. Supernatant was then passed through a 0.2 μm syringe filter and analyzed for Tramadol concentration using UV-Visible Spectroscopy at 271 nm (UV-1700, Shimadzu).

| | | | | | | [Tramadol | |
|---|---|---|---|---|---|---|---|
| | | | | | [Tramadol | HCl] | |
| | | | | | HCl] in | in solution | % of total |
| | | | | | starting | after addition | Tramadol |
| | Charcoal, | [Charcoal], | Charcoal/Tramadol, | abs@ | solution, | of charcoal, | adsorbed to |
| Example | mg | mg/ml | mg/mg | 271 nm | μg/ml | μg/ml | charcoal |
| 1 | 0 | 0 | 0:1 | 0.1490 | 24.02 | 24.02 | 0.00 |
| 2 | 1 | 0.1 | 4:1 | 0.0968 | 24.02 | 15.31 | 36.26 |
| 3 | 2 | 0.2 | 8:1 | 0.0681 | 24.02 | 10.54 | 56.12 |
| 4 | 4 | 0.4 | 16:1 | 0.0374 | 24.02 | 5.42 | 77.45 |
| 5 | 8 | 0.8 | 32:1 | 0.0045 | 24.02 | −0.07 | 100.30 |
| 6 | 10 | 1 | 40:1 | 0.0005 | 24.02 | −0.74 | 103.08 |
| 7 | 20 | 2 | 80:1 | −0.0011 | 24.02 | −0.99 | 104.14 |
| 8 | 40 | 4 | 160:1 | −0.0009 | 24.02 | −0.97 | 104.02 |

FIG. 49 illustrates effective adsorption of Tramadol into charcoal particles.

Effect of Charcoal Granulation (Aggregation)

Charcoal granules were prepared by first wetting 3 g of dry charcoal powder with 8 g of a 2% w/w hypromellose solution in water. Then, 5 g of a 2.5% w/w aqueous Kollidon VA64 solution in water was added and thoroughly mixed to a uniform consistency. The wet mass produced was then passed through a #35 sieve, and the resultant granules dried under hot air at 68° C. Dried granules were then screened for a particle size range of 500-850 μm. A 10 ml volume of 25 μg/ml Tramadol HCl aqueous solution was then added to 20 mg of granules. The sample was vortexed for 5 sec and centrifuged at 1500 rpm for 5 min. Supernatant was passed through a 0.2 μm syringe filter, and analyzed for Tramadol concentration using UV-Visible Spectroscopy at 271 nm (UV-1700, Shimadzu). Additionally, the effect of Tramadol adsorption when the granules (aggregates) were reduced in particle size (crushed) was also measured. Charcoal granules were crushed using a glass mortar and pestle with 40 mg of sample triturated 50 times in a clock-wise direction. A 20 mg sample of the crushed product was used for testing.

| | | Charcoal | | | | |
|---|---|---|---|---|---|---|
| | | particle | | | | |
| | | size | | | | |
| | Charcoal, | range, | Charcoal/Tramadol, | | [Tramadol HCl], | % of drug |
| Example | mg | μm | mg/mg | abs@271 nm | μg/ml | adsorbed |
| 9 | 0 | — | 0:1 | 0.1536 | 24.78 | 0 |
| 10 | 20 | 500-850 | 80:1 | 0.1533 | 24.74 | 0.16 |
| 11 | 20 | 500-850 | 80:1 | 0.082 | 12.85 | 48.15 |
| | (crushed) | | | | | |

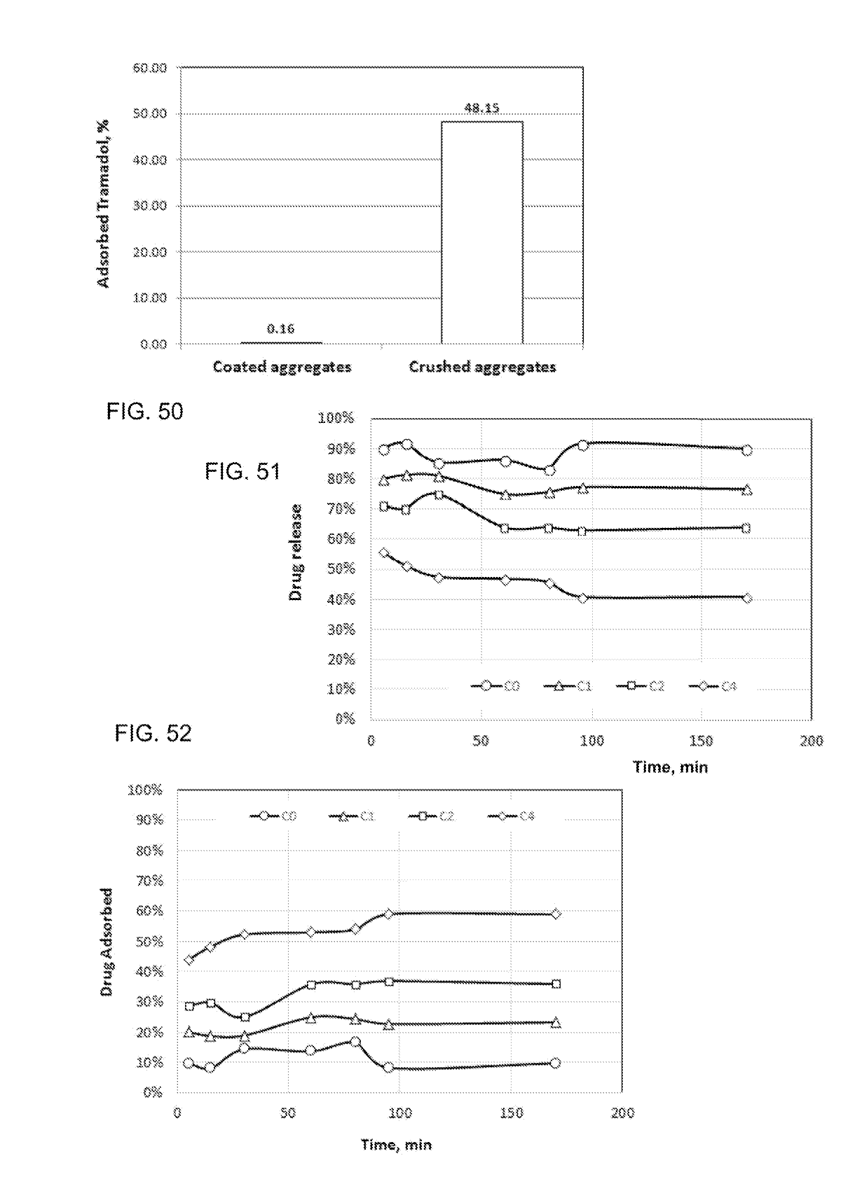

FIG. 50 illustrates the effect of coating on Tramadol adsorption into charcoal aggregates.

Charcoal Adsorption of Tramadol HCl in Tablets

Effect of Charcoal Concentration

Charcoal was formulated into tablets using four different formulas of differing charcoal content. Tablets were made on a single station carver press at a compression force of approximately 1000 pounds using a 7/16″ punch and die. Tablets having a composition of materials over 500 mg were made by dividing the powder and punching into separate tablets. Dissolution studies were then performed for each composition using a USP 2 Paddle method (Distek dissolution system 2100A) in 900 ml of ultrapure water at 37.5° C. at a paddle rotational speed of 50 rpm. After 80 minutes, the dissolution medium was changed to 0.1N HCl by the addition of concentrated hydrochloric acid into the dissolution medium. Tramadol HCl concentration in the dissolution medium was analyzed using UV-Visible Spectroscopy at 271 nm (UV-1700, Shimadzu) over time.

Tablet Formulations:

Tablet formulations containing different compositions of Tramadol and charcoal:

| | | | | Prosolv | | | |
|---|---|---|---|---|---|---|---|
| | | Tramadol | | SMCC | Polyplasdone | Calculated | Actual |
| | Charcoal/Tramadol | HCl, | Charcoal, | 90, | XL, | weight, | Weight, |
| Formula | ratio | mg | mg | mg | mg | mg | mg |
| C0 | 0:1 | 25 | 0 | 150 | 50 | 225 | 221.5 |
| C1 | 2:1 | 25 | 50 | 100 | 50 | 225 | 225 |
| C2 | 4:1 | 25 | 100 | 200 | 100 | 425 | 423.7 |
| C4 | 8:1 | 25 | 200 | 400 | 200 | 825 | 828.3 |

Dissolution profiles of the tablet formulations prepared as in formulations above:

| | | | | | | % of |
|---|---|---|---|---|---|---|
| Exam- | | Dissolution | Time, | abs@ | [Tramadol | drug |
| ple | Formula | Medium | min | 271 nm | HCl], μg/ml | released |
| 12 | C0 | Water | 5 | 0.1554 | 25.08 | 90.28 |
| 13 | C0 | Water | 15 | 0.1580 | 25.52 | 91.88 |
| 14 | C0 | Water | 30 | 0.1476 | 23.78 | 85.60 |
| 15 | C0 | Water | 60 | 0.1487 | 23.97 | 86.30 |
| 16 | C0 | Water | 80 | 0.1439 | 23.16 | 83.38 |
| 17 | C0 | 0.1N HCl | 95 | 0.1578 | 25.48 | 91.74 |
| 18 | C0 | 0.1N HCl | 170 | 0.1554 | 25.08 | 90.28 |
| 19 | C1 | Water | 5 | 0.1381 | 22.20 | 79.92 |
| 20 | C1 | Water | 15 | 0.1403 | 22.57 | 81.24 |
| 21 | C1 | Water | 30 | 0.1400 | 22.52 | 81.08 |
| 22 | C1 | Water | 60 | 0.1299 | 20.84 | 75.02 |
| 23 | C1 | Water | 80 | 0.1309 | 21.00 | 75.58 |
| 24 | C1 | 0.1N HCl | 95 | 0.1336 | 21.46 | 77.24 |
| 25 | C1 | 0.1N HCl | 170 | 0.1328 | 21.32 | 76.74 |
| 26 | C2 | Water | 5 | 0.1237 | 19.81 | 71.30 |
| 27 | C2 | Water | 15 | 0.1219 | 19.51 | 70.22 |
| 28 | C2 | Water | 30 | 0.1299 | 20.83 | 74.98 |
| 29 | C2 | Water | 60 | 0.1118 | 17.82 | 64.14 |
| 30 | C2 | Water | 80 | 0.1117 | 17.80 | 64.08 |
| 31 | C2 | 0.1N HCl | 95 | 0.1100 | 17.52 | 63.06 |
| 32 | C2 | 0.1N HCl | 170 | 0.1116 | 17.79 | 64.04 |
| 33 | C4 | Water | 5 | 0.0982 | 15.54 | 55.96 |
| 34 | C4 | Water | 15 | 0.0911 | 14.37 | 51.72 |
| 35 | C4 | Water | 30 | 0.0844 | 13.25 | 47.70 |
| 36 | C4 | Water | 60 | 0.0832 | 13.05 | 46.98 |
| 37 | C4 | Water | 80 | 0.0816 | 12.78 | 46.02 |
| 38 | C4 | 0.1N HCl | 95 | 0.0732 | 11.38 | 40.98 |
| 39 | C4 | 0.1N HCl | 170 | 0.0733 | 11.39 | 41.02 |

FIGS. 51 and 52 illustrate release and adsorption profiles of the tablet formulations containing different Tramadol charcoal compositions.

Abuse Studies

Effect of pH

A 10 ml volume of 25 μg/ml Tramadol HCl aqueous solution made of different molar concentrations of HCl was added to 10 mg of charcoal. Samples were vortexed for 5 sec, and then centrifuged at 1500 rpm for 5 min. Supernatant was then passed through a 0.2 μm syringe filter, and analyzed for Tramadol concentration using UV-Visible Spectroscopy at 271 nm (UV-1700, Shimadzu).

| | | | | | | [Tramadol | |
|---|---|---|---|---|---|---|---|
| | | | | | | HCl] in | |
| | | | abs@ | abs@ | [Tramadol | solution | |
| | | | 271 nm of | 271 nm | HCl] in | after | % of total |
| | | | Tramadol | after | starting | charcoal | Tramadol |
| | Charcoal, | Acid | starting | addition of | solution, | addition, | adsorbed |
| Example | mg | concentration, N | solution | Charcoal | μg/ml | μg/ml | to charcoal |
| 40 | 10 | 0.1 | 0.1479 | 0.0035 | 23.83 | −0.23 | 100.98 |
| 41 | 10 | 0.01 | 0.1460 | 0.0050 | 23.51 | 0.02 | 99.93 |
| 42 | 10 | 0.001 | 0.1473 | 0.0079 | 23.73 | 0.51 | 97.87 |
| 43 | 10 | 0.0001 | 0.1495 | 0.0085 | 24.11 | 0.59 | 97.53 |
| 44 | 10 | 0.00001 | 0.1457 | 0.0021 | 23.46 | −0.47 | 102.01 |

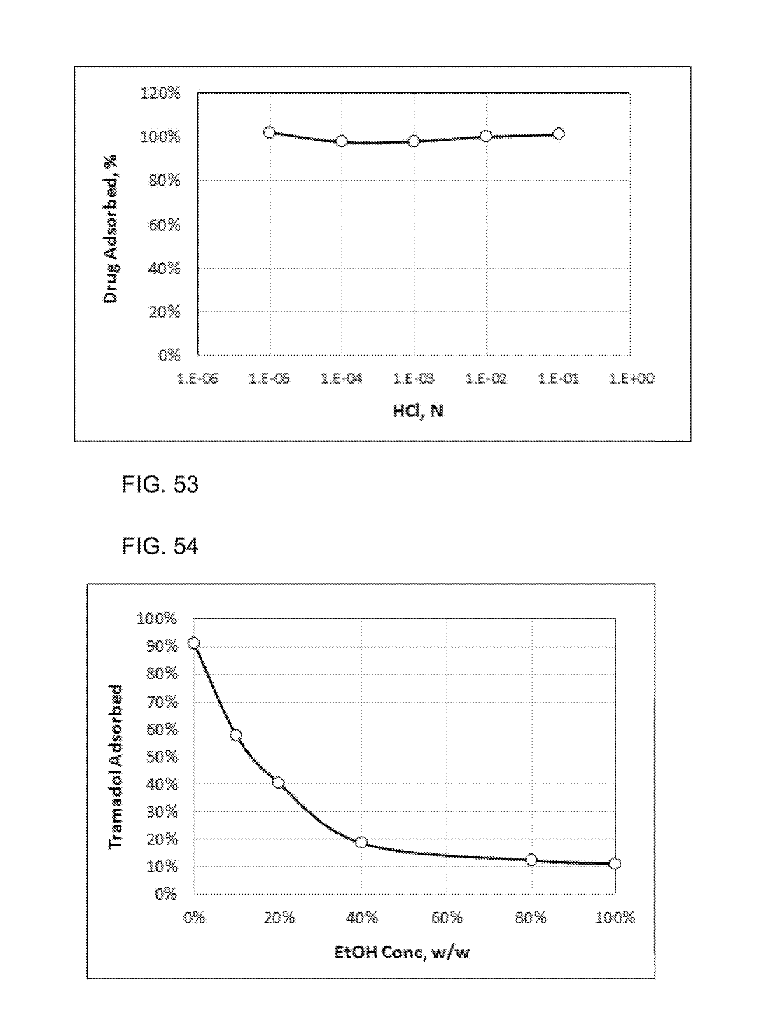

FIG. 53 illustrates the effect of pH on charcoal Tramadol adsorption.

Effect of Alcohol

A 10 ml volume of 25 μg/ml Tramadol HCl in various hydroalcoholic concentrations was added to 10 mg of charcoal. Samples were vortexed for 5 sec, and then centrifuged at 1500 rpm for 5 min. Supernatant was then passed through a 0.2 μm syringe filter and analyzed for Tramadol concentration using UV-Visible Spectroscopy at 271 nm (UV-1700, Shimadzu).

| | | | | | | [Tramadol | |
|---|---|---|---|---|---|---|---|
| | | | | abs@ | | HCl] in | |
| | | | abs@ | 271 nm | [Tramadol | solution | |
| | | | 271 nm of | after | HCl] in | after | % of total |
| | | | Tramadol | addition | starting | charcoal | Tramadol |
| | Charcoal, | EtOH, | starting | of 10 mg | solution, | addition, | adsorbed |
| Example | mg | % w/w | solution | charcoal | μg/ml | μg/ml | to charcoal |
| 45 | 10 | 0% | 0.1484 | 0.0179 | 23.91 | 2.17 | 90.94 |
| 46 | 10 | 10% | 0.1408 | 0.0623 | 22.65 | 9.57 | 57.76 |
| 47 | 10 | 20% | 0.1504 | 0.0914 | 24.26 | 14.42 | 40.54 |
| 48 | 10 | 40% | 0.1512 | 0.1241 | 24.39 | 19.87 | 18.54 |
| 49 | 10 | 80% | 0.1638 | 0.1429 | 24.74 | 21.66 | 12.43 |
| 50 | 10 | 100% | 0.1608 | 0.1423 | 24.29 | 21.57 | 11.22 |

FIG. 54 illustrates the effect of alcohol on charcoal adsorption of Tramadol HCl

Combined Deterrent Agents

In an embodiment of the disclosure, an effective combination of three powerful deterrent agents, crosslinked carboxymethylcellulose, bentonite clay, and medicinal charcoal, can effectively bind to Tramadol HCl in five solutions including pure water, 0.9% saline, 40% aqueous ethyl alcohol (EtOH 40%), a pH 3 solution, and 0.1N HCl. This embodiment provides an effective trapping effect of the deterrent mix in all first four solutions; however the trapping effect is not as great for 0.1N HCl. We used a matrix design of 14 experiments, and used MINITAB software, in order to maximize the deterrence capacity of the three deterrents and to optimize their effect when exposed to 0.1N HCl. In our previous study, we had found that a total of 200 mg of deterrent agent can successfully and effectively bind to the drug formulated into an immediate release tablet (containing 25 mg active for instance). However, embodiments herein can bind higher amounts of deterrent in the dosage form, and can provide greater amounts of drug in the dosage form, and can be used with other modes of drug release, such as extended, or sustained release.

Experiments

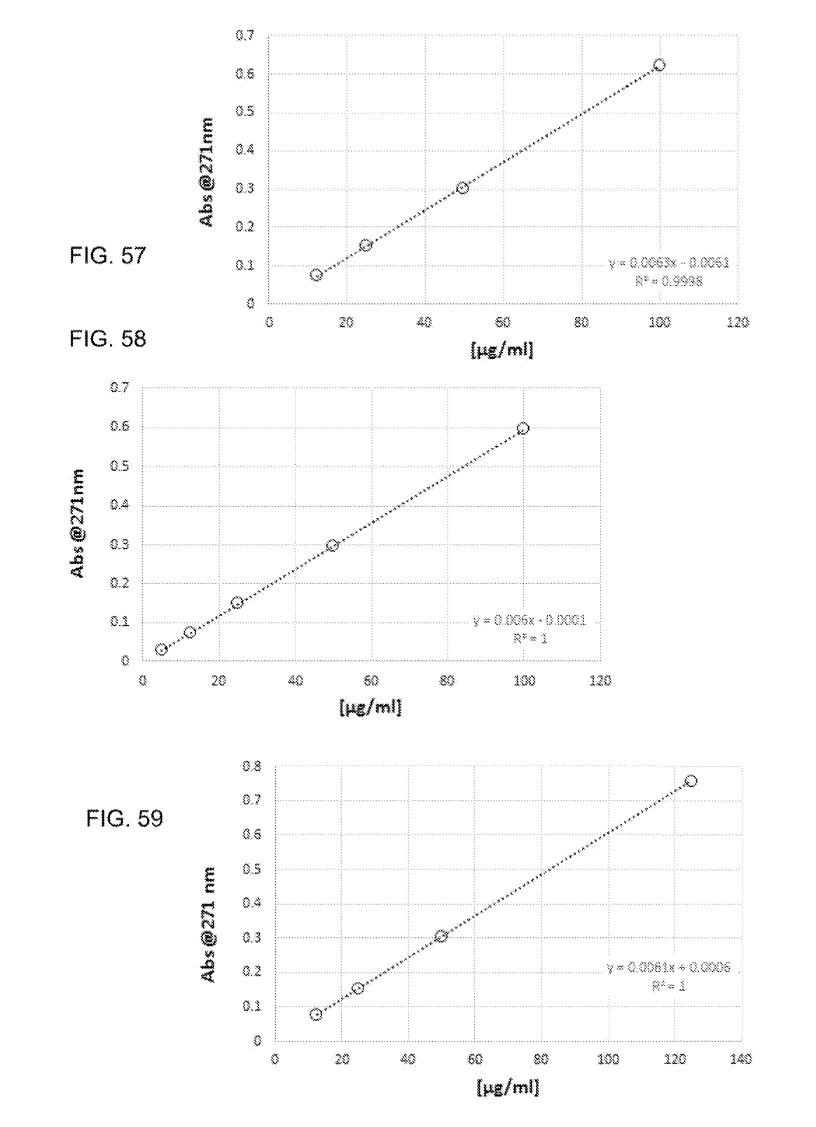

Calibration curve for Full range studies in water (plotted in FIG. 57):

| Conc. μg/ml | Abs1 | Abs2 | Abs3 | Average |
|---|---|---|---|---|
| 12.5 | 0.0759 | 0.073 | 0.0732 | 0.0740 |
| 25 | 0.1525 | 0.1506 | 0.151 | 0.1511 |
| 50 | 0.303 | 0.3014 | 0.303 | 0.3019 |
| 100 | 0.6195 | 0.6234 | 0.6232 | 0.6224 |

Calibration curve for Full range studies in 0.1 N HCl (plotted in FIG. 58):

| Conc. μg/ml | Abs1 | Abs2 | Abs3 | Average |
|---|---|---|---|---|
| 5 | 0.0287 | 0.0289 | 0.0294 | 0.0290 |
| 12.5 | 0.0737 | 0.0746 | 0.0748 | 0.0742 |
| 25 | 0.1506 | 0.149 | 0.1494 | 0.1497 |
| 50 | 0.2974 | 0.2979 | 0.2974 | 0.2976 |
| 100 | 0.5951 | 0.5962 | 0.5946 | 0.5953 |

Calibration curve for Full range studies in 0.9% NS (plotted in FIG. 59):

| Conc. | | | | |
|---|---|---|---|---|
| μg/ml | Abs1 | Abs2 | Abs3 | Average |
| 12.5 | 0.0768 | 0.0769 | 0.0767 | 0.0768 |
| 25 | 0.1511 | 0.1515 | 0.1514 | 0.1513 |
| 50 | 0.3042 | 0.3038 | 0.3043 | 0.3041 |
| 125 | 0.7581 | 0.7584 | 0.7584 | 0.7583 |

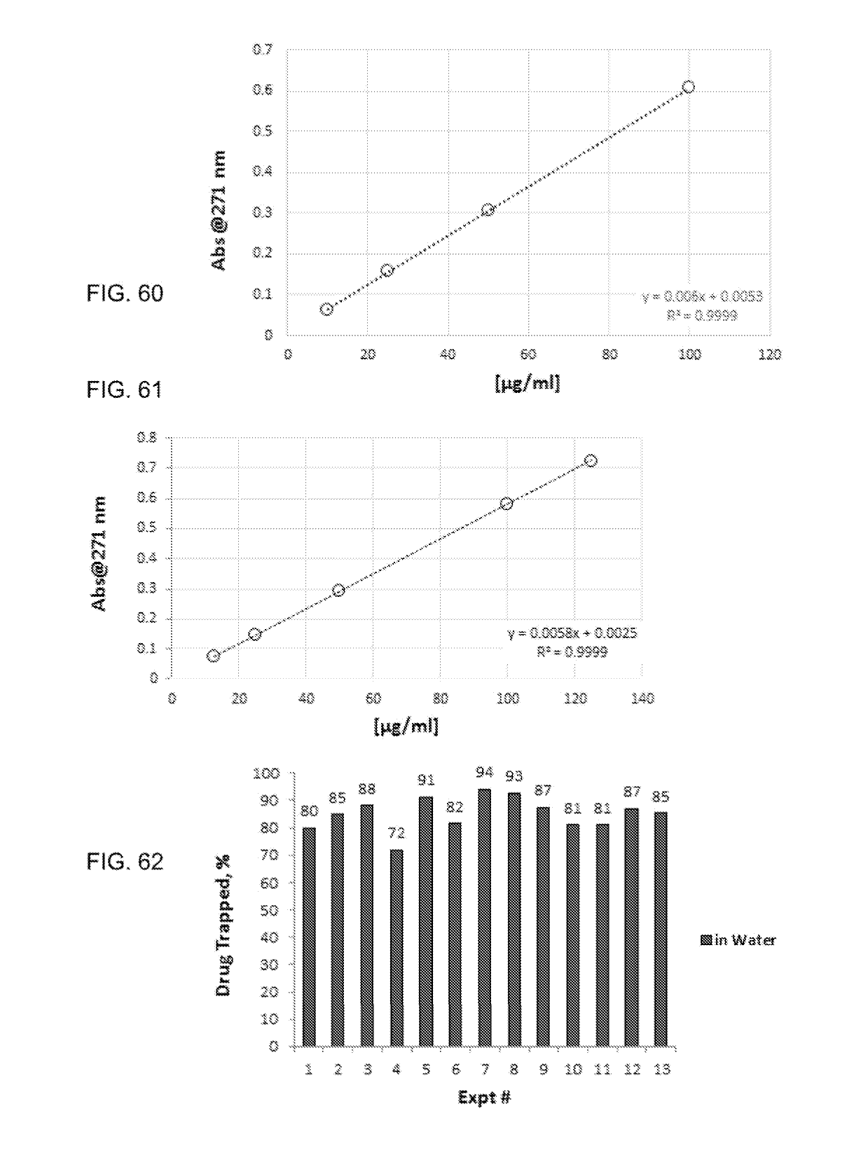

Calibration curve for Full range studies in EtOH 40% (plotted in FIG. 60):

| Conc. μg/ml | Abs1 | Abs2 | Abs3 | Average |
|---|---|---|---|---|
| 10 | 0.0636 | 0.0634 | 0.0636 | 0.0635 |
| 25 | 0.1593 | 0.1576 | 0.1589 | 0.1586 |
| 50 | 0.3065 | 0.3071 | 0.3066 | 0.3067 |
| 100 | 0.6077 | 0.6079 | 0.6079 | 0.6078 |

Calibration curve for Full range studies in pH 3 solution (plotted in FIG. 61):

| Conc. | | | | |
|---|---|---|---|---|
| μg/ml | Abs1 | Abs2 | Abs3 | Average |
| 12.5 | 0.073 | 0.0729 | 0.0725 | 0.0728 |
| 25 | 0.1475 | 0.1475 | 0.1475 | 0.1475 |
| 50 | 0.2935 | 0.2936 | 0.2937 | 0.2936 |
| 100 | 0.5823 | 0.5829 | 0.5828 | 0.5827 |
| 125 | 0.7233 | 0.7233 | 0.7233 | 0.7233 |

Extraction Studies

Using Minitab matrix design of experiment (DOE), total of 14 compositions (200 mg) were prepared containing different amounts of A (AcDiSol), B (Bentonite clay), and C (Charcoal) that were mixed with 25 mg of Tramadol HCl.

Extraction studies in water after 10 min (plotted in FIG. 62):

| | | | | Total | | [Tramadol] | |
|---|---|---|---|---|---|---|---|
| | A, | B, | C, | mix, | UV Abs | in extract, | % Drug |
| Expt # | mg | mg | mg | mg | @271 nm | mg | Trapped |
| 1 | 0.0 | 0.0 | 200.0 | 200.0 | 0.1523 | 5.03 | 79.89 |
| 2 | 66.7 | 133.3 | 0.0 | 200.0 | 0.1128 | 3.77 | 84.91 |
| 3 | 33.3 | 33.3 | 133.3 | 200.0 | 0.0867 | 2.94 | 88.22 |
| 4 | 200.0 | 0.0 | 0.0 | 200.0 | 0.2142 | 6.99 | 72.03 |
| 5 | 0.0 | 66.7 | 133.3 | 200.0 | 0.0634 | 2.21 | 91.18 |
| 6 | 133.3 | 33.3 | 33.3 | 200.0 | 0.1397 | 4.63 | 81.49 |
| 7 | 0.0 | 133.3 | 66.7 | 200.0 | 0.0409 | 1.49 | 94.03 |
| 8 | 0.0 | 200.0 | 0.0 | 200.0 | 0.0518 | 1.84 | 92.65 |
| 9 | 66.7 | 66.7 | 66.7 | 200.0 | 0.0929 | 3.14 | 87.43 |
| 10 | 133.3 | 0.0 | 66.7 | 200.0 | 0.1426 | 4.72 | 81.12 |
| 11 | 133.3 | 66.7 | 0.0 | 200.0 | 0.1432 | 4.74 | 81.04 |
| 12 | 33.3 | 133.3 | 33.3 | 200.0 | 0.0952 | 3.21 | 87.14 |
| 13 | 66.7 | 0.0 | 133.3 | 200.0 | 0.1095 | 3.67 | 85.32 |
| 14 | Blank | Blank | Blank | 0 | 0.7154 | 22.91 | 8.38 |

Notes:
200 mg “A” provided minimum drug trapped of 72% (Expt 4); 133 mg “B” and 66 mg “C” provided maximum drug trapped of 94% (Expt 7); 200 mg “B” provided maximum drug trapped of 93% (Expt 8); and 133 mg “C” and 66 mg “B” provided maximum drug trapped of 91% (Expt 5).

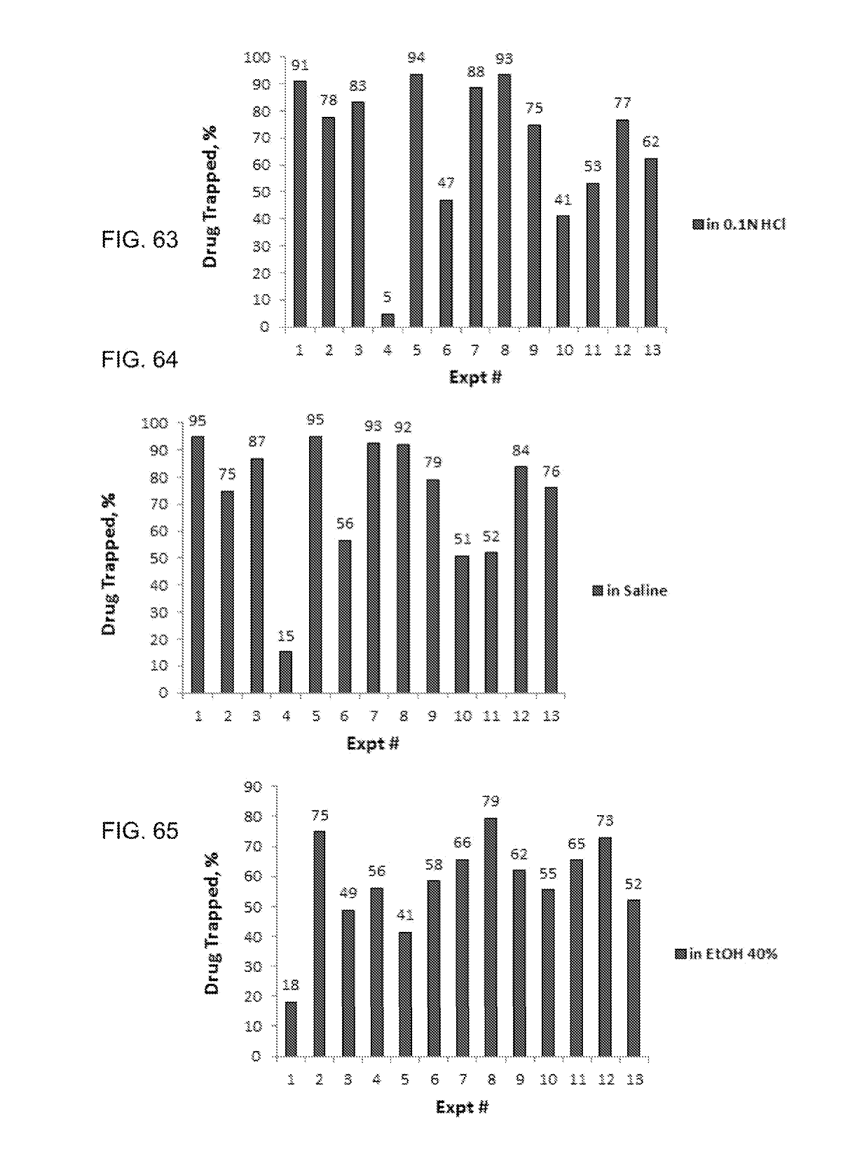

Extraction studies in 0.1N HCl after 10 min (plotted in FIG. 63):

| | | | | Total | UV | [Tramadol] | |
|---|---|---|---|---|---|---|---|
| | A, | B, | C, | mix, | Abs@ | in extract, | % Drug |
| Expt # | mg | mg | mg | mg | 271 nm | mg | Trapped |
| 1 | 0.0 | 0.0 | 200.0 | 200.0 | 0.0684 | 2.28 | 90.87 |
| 2 | 66.7 | 133.3 | 0.0 | 200.0 | 0.1689 | 5.63 | 77.47 |
| 3 | 33.3 | 33.3 | 133.3 | 200.0 | 0.1261 | 4.21 | 83.17 |
| 4 | 200.0 | 0.0 | 0.0 | 200.0 | 0.7153 | 23.85 | 4.61 |
| 5 | 0.0 | 66.7 | 133.3 | 200.0 | 0.0485 | 1.62 | 93.52 |
| 6 | 133.3 | 33.3 | 33.3 | 200.0 | 0.3981 | 13.27 | 46.90 |
| 7 | 0.0 | 133.3 | 66.7 | 200.0 | 0.0871 | 2.91 | 88.37 |
| 8 | 0.0 | 200.0 | 0.0 | 200.0 | 0.0502 | 1.68 | 93.30 |
| 9 | 66.7 | 66.7 | 66.7 | 200.0 | 0.1907 | 6.36 | 74.56 |
| 10 | 133.3 | 0.0 | 66.7 | 200.0 | 0.4429 | 14.77 | 40.93 |
| 11 | 133.3 | 66.7 | 0.0 | 200.0 | 0.3522 | 11.74 | 53.03 |
| 12 | 33.3 | 133.3 | 33.3 | 200.0 | 0.1745 | 5.82 | 76.72 |
| 13 | 66.7 | 0.0 | 133.3 | 200.0 | 0.2830 | 9.44 | 62.25 |
| 14 | Blank | Blank | Blank | 0 | 0.7496 | 24.99 | 0.04 |

Notes:
200 mg “A” provided minimum drug trapped of 5% (Expt 4); 133 mg “C” and 66 mg “B” provided maximum drug trapped of 94% (Expt 5); 200 mg “B” provided maximum drug trapped of 93% (Expt 8); and 200 mg “C” provided maximum drug trapped of 91% (Expt 1).

Extraction studies in 0.9% NS after 10 min (plotted in FIG. 64):

| | | | | Total | UV | [Tramadol] | |
|---|---|---|---|---|---|---|---|
| | A, | B, | C, | mix, | Abs@ | in extract, | % Drug |
| Expt # | mg | mg | mg | mg | 271 nm | mg | Trapped |
| 1 | 0.0 | 0.0 | 200.0 | 200.0 | 0.0412 | 1.33 | 94.68 |
| 2 | 66.7 | 133.3 | 0.0 | 200.0 | 0.1919 | 6.27 | 74.91 |
| 3 | 33.3 | 33.3 | 133.3 | 200.0 | 0.1017 | 3.32 | 86.74 |
| 4 | 200.0 | 0.0 | 0.0 | 200.0 | 0.6487 | 21.25 | 15.01 |
| 5 | 0.0 | 66.7 | 133.3 | 200.0 | 0.0392 | 1.26 | 94.94 |
| 6 | 133.3 | 33.3 | 33.3 | 200.0 | 0.3328 | 10.89 | 56.43 |
| 7 | 0.0 | 133.3 | 66.7 | 200.0 | 0.0566 | 1.84 | 92.66 |
| 8 | 0.0 | 200.0 | 0.0 | 200.0 | 0.0627 | 2.03 | 91.86 |
| 9 | 66.7 | 66.7 | 66.7 | 200.0 | 0.1588 | 5.19 | 79.25 |
| 10 | 133.3 | 0.0 | 66.7 | 200.0 | 0.3776 | 12.36 | 50.55 |
| 11 | 133.3 | 66.7 | 0.0 | 200.0 | 0.3670 | 12.01 | 51.95 |
| 12 | 33.3 | 133.3 | 33.3 | 200.0 | 0.1251 | 4.08 | 83.67 |
| 13 | 66.7 | 0.0 | 133.3 | 200.0 | 0.1843 | 6.02 | 75.91 |
| 14 | Blank | Blank | Blank | 0 | 0.7550 | 24.73 | 1.06 |

Notes:
200 mg “A” provided minimum drug trapped of 15% (Expt 4); 200 mg “C” provided maximum drug trapped of 95% (Expt 1); 133 mg “C” and 66 mg “B” provided maximum drug trapped of 95% (Expt 5); 133 mg “B” and 66 mg “C” provided maximum drug trapped of 93% (Expt 7); and 200 mg “B” provided maximum drug trapped of 92% (Expt 8).

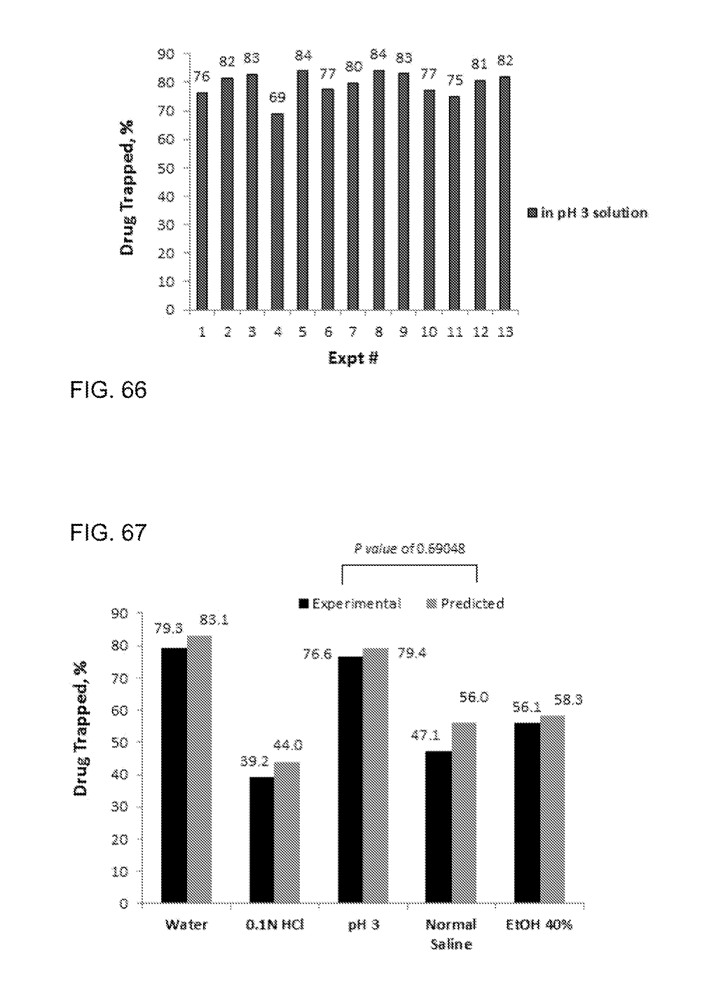

Extraction studies in 40% EtOH after 10 min (plotted in FIG. 65):

| | | | | Total | UV | [Tramadol] | |
|---|---|---|---|---|---|---|---|
| | A, | B, | C, | mix, | Abs@ | in extract, | % Drug |
| Expt # | mg | mg | mg | mg | 271 nm | mg | Trapped |
| 1 | 0.0 | 0.0 | 200.0 | 200.0 | 0.6200 | 20.49 | 18.04 |
| 2 | 66.7 | 133.3 | 0.0 | 200.0 | 0.1945 | 6.31 | 74.77 |
| 3 | 33.3 | 33.3 | 133.3 | 200.0 | 0.3900 | 12.82 | 48.70 |
| 4 | 200.0 | 0.0 | 0.0 | 200.0 | 0.3358 | 11.02 | 55.94 |
| 5 | 0.0 | 66.7 | 133.3 | 200.0 | 0.4454 | 14.67 | 41.32 |
| 6 | 133.3 | 33.3 | 33.3 | 200.0 | 0.3176 | 10.41 | 58.36 |
| 7 | 0.0 | 133.3 | 66.7 | 200.0 | 0.2643 | 8.63 | 65.47 |
| 8 | 0.0 | 200.0 | 0.0 | 200.0 | 0.1601 | 5.16 | 79.36 |
| 9 | 66.7 | 66.7 | 66.7 | 200.0 | 0.2911 | 9.53 | 61.89 |
| 10 | 133.3 | 0.0 | 66.7 | 200.0 | 0.3395 | 11.14 | 55.44 |
| 11 | 133.3 | 66.7 | 0.0 | 200.0 | 0.2656 | 8.68 | 65.29 |
| 12 | 33.3 | 133.3 | 33.3 | 200.0 | 0.2091 | 6.79 | 72.83 |
| 13 | 66.7 | 0.0 | 133.3 | 200.0 | 0.3656 | 12.01 | 51.96 |
| 14 | Blank | Blank | Blank | 0 | 0.7924 | 26.24 | −4.95 |

Notes:
200 mg “C” provided minimum drug trapped of 18% (Expt 1); 200 mg “B” provided maximum drug trapped of 79% (Expt 8); 133 mg “B” and 66 mg “A” provided maximum drug trapped of 75% (Expt 2); and 133 mg “B” and 33 mg “A” and 33 mg “C” provided maximum drug trapped of 73% (Expt 12).

Extraction studies in pH 3 solution after 10 min (plotted in FIG. 66):

| | | | | Total | UV | Tramadol | |
|---|---|---|---|---|---|---|---|
| | A, | B, | C, | mix, | Abs@ | in extract, | % Drug |
| Expt # | mg | mg | mg | mg | 271 nm | mg | Trapped |
| 1 | 0.0 | 0.0 | 200.0 | 200.0 | 0.173033 | 5.88 | 76.19 |
| 2 | 66.7 | 133.3 | 0.0 | 200.0 | 0.1406 | 4.76 | 81.66 |
| 3 | 33.3 | 33.3 | 133.3 | 200.0 | 0.131633 | 4.45 | 82.71 |
| 4 | 200.0 | 0.0 | 0.0 | 200.0 | 0.240767 | 8.22 | 69.00 |
| 5 | 0.0 | 66.7 | 133.3 | 200.0 | 0.1157 | 3.90 | 83.86 |
| 6 | 133.3 | 33.3 | 33.3 | 200.0 | 0.174933 | 5.95 | 77.35 |
| 7 | 0.0 | 133.3 | 66.7 | 200.0 | 0.1563 | 5.30 | 79.90 |
| 8 | 0.0 | 200.0 | 0.0 | 200.0 | 0.1324 | 4.48 | 84.20 |
| 9 | 66.7 | 66.7 | 66.7 | 200.0 | 0.1371 | 4.64 | 83.07 |
| 10 | 133.3 | 0.0 | 66.7 | 200.0 | 0.171367 | 5.82 | 77.30 |
| 11 | 133.3 | 66.7 | 0.0 | 200.0 | 0.193733 | 6.59 | 75.03 |
| 12 | 33.3 | 133.3 | 33.3 | 200.0 | 0.154667 | 5.25 | 80.78 |
| 13 | 66.7 | 0.0 | 133.3 | 200.0 | 0.139167 | 4.71 | 82.03 |
| 14 | Blank | Blank | Blank | 0 | 0.725367 | 24.93 | 0.29 |

Notes:
200 mg “A” provided minimum drug trapped of 69% (Expt 4); 200 mg “B” provided maximum drug trapped of 84% (Expt 8); 133 mg of “C” and 66 mg of “B” provided maximum drug trapped of 84% (Expt 5); 133 mg “C”, 33 mg “A” and 33 mg “B” provided maximum drug trapped of 83% (Expt 3); 133 mg “B” and 66 mg “A” provided maximum drug trapped of 82% (Expt 2); and 133 mg “C” and 66 mg “A” provided maximum drug trapped of 82% (Expt 13).

Summary of Extraction Studies

| | | | | | | | % Drug | % Drug | % Drug |
|---|---|---|---|---|---|---|---|---|---|
| | | | | % Drug | % Drug | % Drug | trapped/ | released/ | trapped/ |
| | A, | B, | C, | trapped/ | trapped/ | trapped/ | pH 3 | pH 1 | pH 1 |
| Expt # | mg | mg | mg | Water | Saline | EtOH40 | solution | solution* | solution |
| 1 | 0.0 | 0.0 | 200.0 | 79.9 | 94.7 | 18.0 | 76.2 | 9.1 | 90.9 |
| 2 | 66.7 | 133.3 | 0.0 | 84.9 | 74.9 | 74.8 | 81.7 | 22.5 | 77.5 |
| 3 | 33.3 | 33.3 | 133.3 | 88.2 | 86.7 | 48.7 | 82.7 | 16.8 | 83.2 |
| 4 | 200.0 | 0.0 | 0.0 | 72.0 | 15.0 | 55.9 | 69.0 | 95.4 | 4.6 |
| 5 | 0.0 | 66.7 | 133.3 | 91.2 | 94.9 | 41.3 | 83.9 | 6.5 | 93.5 |
| 6 | 133.3 | 33.3 | 33.3 | 81.5 | 56.4 | 58.4 | 77.4 | 53.1 | 46.9 |
| 7 | 0.0 | 133.3 | 66.7 | 94.0 | 92.7 | 65.5 | 79.9 | 11.6 | 88.4 |
| 8 | 0.0 | 200.0 | 0.0 | 92.6 | 91.9 | 79.4 | 84.2 | 6.7 | 93.3 |
| 9 | 66.7 | 66.7 | 66.7 | 87.4 | 79.2 | 61.9 | 83.1 | 25.4 | 74.6 |
| 10 | 133.3 | 0.0 | 66.7 | 81.1 | 50.6 | 55.4 | 77.3 | 59.1 | 40.9 |
| 11 | 133.3 | 66.7 | 0.0 | 81.0 | 51.9 | 65.3 | 75.0 | 47.0 | 53.0 |
| 12 | 33.3 | 133.3 | 33.3 | 87.1 | 83.7 | 72.8 | 80.8 | 23.3 | 76.7 |
| 13 | 66.7 | 0.0 | 133.3 | 85.3 | 75.9 | 51.9 | 82.0 | 37.8 | 62.2 |
| 14 | Blank | Blank | Blank | 8.4 | 1.1 | −4.9 | 0.3 | | 0.0 |

*Data for this column was obtained by subtracting the last column data from 100, giving the amount of drug released in 0.1N HCl.

DOE Results

We assumed that the composition will be used as coated to avoid its interaction with the 0.1N HCl solution. In other words, if the medication is taken by a regular patient orally, the deterrent composition must remain ineffective. However, the effect needs to be maximized in all extracting solutions if the medication is intentionally tampered. Goal maximum was characterized with lower value of 50 and upper value of 100, where the target value was set at 100.

DOE Response for individual and combined extracting solutions

| | AcDiSol | Bentonite | Charcoal | DOE Response | Desirability |
|---|---|---|---|---|---|
| Water | 0 | 141.4 | 58.6 | 93.8 | 0.875 |
| EtOH 40% | 0 | 200 | 0 | 80.7 | 0.613 |
| Saline | 0 | 44.9 | 155.0 | 94.5 | 0.889 |
| pH 3 solution | 20.3 | 103.0 | 76.7 | 81.9 | 0.638 |
| Water | 0 | 200 | 0 | 91.6 | 0.831 |
| EtOH 40% | | | | 80.7 | 0.613 |
| | | | | | 0.714 (composite) |
| Water | 0 | 121.2 | 78.8 | 93.5 | 0.869 |
| Saline | | | | 93.4 | 0.869 |
| | | | | | 0.869 (composite) |
| Water | 0 | 139.4 | 60.6 | 93.8 | 0.875 |
| pH 3 solution | | | | 81.8 | 0.635 |
| | | | | | 0.746 (composite) |
| EtOH 40% | 0 | 200 | 0 | 80.7 | 0.613 |
| Saline | | | | 90.2 | 0.804 |
| | | | | | 0.702 (composite) |
| EtOH 40% | 0 | 200 | 0 | 80.7 | 0.613 |
| pH 3 solution | | | | 80.9 | 0.617 |
| | | | | | 0.615 (composite) |
| Saline | 0 | 100 | 100 | 93.9 | 0.878 |
| pH 3 solution | | | | 81.5 | 0.631 |
| | | | | | 0.744 (composite) |
| Water | 0 | 200 | 0 | 91.6 | 0.831 |
| EtOH 40% | | | | 80.7 | 0.613 |
| Saline | | | | 90.2 | 0.804 |
| | | | | | 0.743 (composite) |
| Water | 0 | 123.2 | 76.8 | 93.5 | 0.871 |
| Saline | | | | 93.4 | 0.867 |
| pH 3 solution | | | | 81.8 | 0.635 |
| | | | | | 0.783 (composite) |
| Water | 0 | 200 | 0 | 91.6 | 0.831 |
| pH 3 solution | | | | 80.8 | 0.617 |
| EtOH 40% | | | | 80.7 | 0.613 |
| | | | | | 0.680 (composite) |
| pH 3 solution | 0 | 200 | 0 | 80.8 | 0.617 |
| EtOH 40% | | | | 80.7 | 0.613 |
| Saline | | | | 90.2 | 0.804 |
| | | | | | 0.673 (composite) |
| pH 3 solution | 0 | 200 | 0 | 80.8 | 0.617 |
| EtOH 40% | | | | 80.7 | 0.613 |
| Saline | | | | 90.2 | 0.804 |
| Water | | | | 91.6 | 0.831 |
| | | | | | 0.709 (composite) |

DOE Response for individual and combined extracting solutions optimized against 0.1N HCl

| Water | 200 | 0 | 0 | 72.6 | 0.45 |
|---|---|---|---|---|---|
| 0.1N HCl | | | | 92.3 | 0.85 |
| | | | | | 0.62 (Composite) |
| Saline | No optimal composition found | | | | |
| 0.1N HCl | | | | | |
| EtOH 40% | 175.8 | 0 | 24.2 | 57.3 | 0.146 |
| 0.1N HCl | | | | 80.2 | 0.605 |
| | | | | | 0.298 (composite) |
| pH 3 solution | 200 | 0 | 0 | 68.4 | 0.367 |
| 0.1N HCl | | | | 92.3 | 0.846 |
| | | | | | 0.56 (composite) |
| Water | 123.2 | 0 | 76.8 | 83.1 | 0.661 |
| Saline | | | | 56.0 | 0.120 |
| 0.1N HCl | | | | 56.0 | 0.120 |
| | | | | | 0.212 (composite) |
| Water | 170.0 | 0 | 30 | 77.8 | 0.557 |
| EtOH 40% | | | | 58.0 | 0.159 |
| 0.1N HCl | | | | 77.3 | 0.546 |
| | | | | | 0.364 (composite) |
| Water | 182 | 0 | 18 | 76.0 | 0.518 |
| pH 3 solution | | | | 71.8 | 0.436 |
| 0.1N HCl | | | | 83.2 | 0.664 |
| | | | | | 0.532 (composite) |
| Saline | 123.2 | 0 | 76.8 | 56.0 | 0.12 |
| EtOH 40% | | | | 58.3 | 0.165 |
| 0.1N HCl | | | | 56.0 | 0.119 |
| | | | | | 0.133 (composite) |
| Saline | No optimal composition found | | | | |
| pH 3 solution | | | | | |
| 0.1N HCl | | | | | |
| EtOH 40% | 170.0 | 0 | 30 | 58.0 | 0.159 |
| pH 3 solution | | | | 73.8 | 0.48 |
| 0.1N HCl | | | | 77.3 | 0.546 |
| | | | | | 0.346 (composite) |
| Water | 163.6 | 0 | 36.4 | 78.7 | 0.574 |
| pH 3 solution | | | | 74.8 | 0.495 |
| EtOH 40% | | | | 58.5 | 0.169 |
| 0.1N HCl | | | | 74.4 | 0.488 |
| | | | | | 0.392 (composite) |
| Water | 123.2 | 0 | 76.8 | 83.1 | 0.661 |
| Saline | | | | 56.0 | 0.120 |
| pH 3 solution | | | | 79.5 | 0.589 |
| 0.1N HCl | | | | 56.0 | 0.120 |
| | | | | | 0.273 (composite) |
| Water | 123.2 | 0 | 76.8 | 83.1 | 0.661 |
| Saline | | | | 56.0 | 0.120 |
| EtOH 40% | | | | 58.3 | 0.165 |
| 0.1N HCl | | | | 56.0 | 0.120 |
| | | | | | 0.199 (composite) |
| Saline | 123.2 | 0 | 76.8 | 56.0 | 0.120 |
| EtOH 40% | | | | 58.3 | 0.165 |
| pH 3 solution | | | | 79.5 | 0.589 |
| 0.1N HCl | | | | 56.0 | 0.120 |
| | | | | | 0.193 (composite) |
| Saline | 123.3 | 0 | 76.7 | 56.0 | 0.120 |
| EtOH 40% | | | | 58.3 | 0.165 |
| pH 3 solution | | | | 79.4 | 0.588 |
| Water | | | | 83.0 | 0.661 |
| 0.1N HCl | | | | 56.0 | 0.120 |
| | | | | | 0.247 (composite) |

DOE Response versus Experimental data for individual and combined extracting solutions

| | | | | DOE | Experi- |
|---|---|---|---|---|---|
| | AcDiSol | Bentonite | Charcoal | Response | mental |
| Water | 0 | 141.4 | 58.6 | 93.8 | 83.97 |
| EtOH 40% | 0 | 200 | 0 | 80.7 | 69.78 |
| Saline | 0 | 44.9 | 155.0 | 94.5 | 87.77 |
| pH 3 solution | 20.3 | 103.0 | 76.7 | 81.9 | 79.97 |
| Water | 0 | 200 | 0 | 91.6 | 92.41 |
| EtOH 40% | | | | 80.7 | 69.78 |
| Water | 0 | 121.2 | 78.8 | 93.5 | 82.05 |
| Saline | | | | 93.4 | 92.24 |
| Water | 0 | 139.4 | 60.6 | 93.8 | 84.66 |
| pH 3 solution | | | | 81.8 | 82.23 |
| EtOH 40% | 0 | 200 | 0 | 80.7 | 69.78 |
| Saline | | | | 90.2 | 84.50 |
| EtOH 40% | 0 | 200 | 0 | 80.7 | 69.78 |
| pH 3 solution | | | | 80.9 | 85.08 |
| Saline | 0 | 100 | 100 | 93.9 | 87.20 |
| pH 3 solution | | | | 81.6 | 81.13 |
| Water | 0 | 200 | 0 | 91.6 | 92.41 |
| EtOH 40% | | | | 80.7 | 69.78 |
| Saline | | | | 90.2 | 84.50 |
| Water | 0 | 123.2 | 76.8 | 93.5 | 85.81 |
| Saline | | | | 93.4 | 90.24 |
| pH 3 solution | | | | 81.8 | 85.54 |
| Water | 0 | 200 | 0 | 91.6 | 92.41 |
| pH 3 solution | | | | 80.8 | 85.08 |
| EtOH 40% | | | | 80.7 | 69.78 |
| pH 3 solution | 0 | 200 | 0 | 80.8 | 85.08 |
| EtOH 40% | | | | 80.7 | 69.78 |
| Saline | | | | 90.2 | 84.50 |
| pH 3 solution | 0 | 200 | 0 | 80.8 | 85.08 |
| EtOH 40% | | | | 80.7 | 69.78 |
| Saline | | | | 90.2 | 84.50 |
| Water | | | | 91.6 | 92.41 |

DOE Response versus Experimental data for individual and combined extracting solutions optimized against 0.1N HCl

| Water | 200 | 0 | 0 | 72.6 | 71.1 |
|---|---|---|---|---|---|
| 0.1N HCl | | | | 92.3 | 96.13 |
| Saline | No optimal composition found | | | | |
| 0.1N HCl | | | | | |
| EtOH 40% | 175.8 | 0 | 24.2 | 57.34 | 59.12 |
| 0.1N HCl | | | | 80.24 | 86.05 |
| pH 3 solution | 200 | 0 | 0 | 68.4 | 66.74 |
| 0.1N HCl | | | | 92.3 | 96.13 |
| Water | 123.2 | 0 | 76.8 | 83.1 | 79.12 |
| Saline | | | | 56.0 | 43.90 |
| 0.1N HCl | | | | 56.0 | 58.73 |
| Water | 170.0 | 0 | 30 | 77.8 | 74.53 |
| EtOH 40% | | | | 58.0 | 58.60 |
| 0.1N HCl | | | | 77.3 | 82.49 |
| Water | 182 | 0 | 18 | 76.0 | 74.08 |
| pH 3 solution | | | | 71.8 | 70.07 |
| 0.1N HCl | | | | 83.2 | 86.85 |
| Saline | 123.2 | 0 | 76.8 | 56.0 | 43.90 |
| EtOH 40% | | | | 58.3 | 55.09 |
| 0.1N HCl | | | | 56.0 | 58.73 |
| Saline | No optimal composition found | | | | |
| pH 3 solution | | | | | |
| 0.1N HCl | | | | | |
| EtOH 40% | 170.0 | 0 | 30 | 58.0 | 58.60 |
| pH 3 solution | | | | 73.9 | 71.26 |
| 0.1N HCl | | | | 77.3 | 82.49 |
| Water | 163.6 | 0 | 36.4 | 78.7 | 75.35 |
| pH 3 solution | | | | 74.8 | 71.52 |
| EtOH 40% | | | | 58.5 | 57.53 |
| 0.1N HCl | | | | 74.4 | 81.22 |
| Water | 123.2 | 0 | 76.8 | 83.1 | 79.12 |
| Saline | | | | 56.0 | 43.90 |
| pH 3 solution | | | | 79.5 | 74.42 |
| 0.1N HCl | | | | 56.0 | 58.73 |
| Water | 123.2 | 0 | 76.8 | 83.1 | 79.12 |
| Saline | | | | 56.0 | 43.90 |
| EtOH 40% | | | | 58.3 | 55.09 |
| 0.1N HCl | | | | 56.0 | 58.73 |
| Saline | 123.2 | 0 | 76.8 | 56.0 | 43.90 |
| EtOH 40% | | | | 58.3 | 55.09 |
| pH 3 solution | | | | 79.5 | 74.42 |
| 0.1N HCl | | | | 56.0 | 58.73 |
| Saline | 123.3 | 0 | 76.7 | 56.0 | 43.90 |
| EtOH 40% | | | | 58.3 | 55.09 |
| pH 3 solution | | | | 79.4 | 74.42 |
| Water | | | | 83.0 | 79.12 |
| 0.1N HCl | | | | 56.0 | 58.73 |

Optimization in five solutions as suggested by DOE and confirmed by Experimental data

Composition Formulation:

| | Experimental, | |
|---|---|---|
| Composition | mg | DOE Suggested, mg |
| Tramadol HCl | 25 | 25 |
| AcDiSol | 124 | 123.34 |
| Charcoal | 76 | 76.66 |

Procedure: Each individual component was weighed and added to 20 mL glass vial. 10 mL of each individual solvent (water, normal saline, 40% ethanol, pH3 and 0.1N HCl) was added to glass vial and vortexed for 5 seconds. The solution was centrifuged for 5 minutes @1500 rpm and filtered after 10 minutes. The supernatant fluid was filtered using 0.2 micron syringe filter and 0.5 mL of filtrate was diluted to 10 mL. The concentration of the drug was determined with help of UV @271 nm. Results are plotted in FIG. 67.

| | Experimental | Predicted values |
|---|---|---|
| | Values of % drug | of % drug |
| Medium | trapped | trapped |
| water | 79.28 | 83.05 |
| 0.1N HCl | 39.19 | 43.97 |
| pH 3 | 76.56 | 79.44 |
| Normal Saline | 47.09 | 55.95 |
| EtOH40% | 56.13 | 58.27 |

As used herein, the term “about” means plus or minus ten (10) percent of the stated numerical value. All patents and publications mentioned in this specification are indicative of the levels of those skilled in the art to which the disclosure pertains. All patents and publications are herein incorporated by reference to the same extent as if each individual publication was specifically and individually indicated to be incorporated by reference. It is to be understood that while a certain form of the disclosure is illustrated, it is not intended to be limited to the specific form or arrangement herein described and shown. It will be apparent to those skilled in the art that various changes may be made without departing from the scope of the disclosure and the disclosure is not to be considered limited to what is shown and described in the specification. One skilled in the art will readily appreciate that the present disclosure is well adapted to carry out the objectives and obtain the ends and advantages mentioned, as well as those inherent therein. The methods, procedures, and techniques described herein are presently representative of the preferred embodiments, are intended to be exemplary and are not intended as limitations on the scope. Changes therein and other uses will occur to those skilled in the art which are encompassed within the disclosure. Although the disclosure has been described in connection with specific, preferred embodiments, it should be understood that the disclosure as ultimately claimed should not be unduly limited to such specific embodiments. Indeed various modifications of the described modes for carrying out the disclosure which are obvious to those skilled in the art are intended to be within the scope of the disclosure.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications might occur to those skilled in the art to which the disclosure pertains. Accordingly, all expedient modifications readily attainable by one versed in the art from the disclosure set forth herein are to be included as further embodiments of the present disclosure.

### BIBLIOGRAPHY AND REFERENCES

1. The national center on addiction and substance abuse (CASA) at Columbia University, Under the counter: the diversion and abuse of controlled prescription drugs in the U.S. 2005: New York, N.Y.
2. Substance abuse and mental health services administration, Results from the 2009 national survey on drug use and health: volume I. summary of national findings. Office of applied studies, NSDUH series H-38A, HHS publication no. SMA 10-4586 Findings, Rockville, Md., 2010.
3. Manchikanti, L., et al., Therapeutic use, abuse, and nonmedical use of opiolds: a ten-year perspective. Pain Physician, 2010. 13(5): p. 401-35.
4. Warner, M., et al., Drug poisoning deaths in the United States, 1980-2008. NCHS data brief, na 81. 2011, National Center for Health Statistics: Hyattsville, Md.
5. Substance abuse and mental health services administration, Treatment episode data set (TEDS): 1998-2008. State admissions to substance abuse treatment services. Center for behavioral health statistics and quality, OASIS series: S-55, HHS publication no. (SMA) 10-4613, Rockville, Md., 2010.
6. Center for Disease Control and Prevention. Prescription Painkiller Overdoses in the U.S. 2011 [cited 2012 14 Feb.]; Available from: http://www.cdc.gov/Features/VitaiSigns/PainkillerOverdoses/.
7. United Nations Office on Drugs and Crime (UNODC), World Drug Report 2011. United Nations Publications, 2011. Sales No. E.11.XI.10.
8. Substance abuse and mental health services administration, Results from the 2008 national survey on drug use and health: national findings. Office of applied studies, NSDUH series H-36, HHS publication no. SMA 09-4434, Rockville, Md., 2009.
9. Johnston, L. D., et al. Monitoring the future, national results on adolescent drug use: overview of key findings, 2010. 2011 [cited 2011 18 Mar.]; Available from: http://www.monitoringthefuture.org/pubs/monographs/mtf-overview2010.pdf
10. Substance abuse and mental health services administration. Center for behavioral health statistics and quality, The DAWN report: drug-related emergency department visits involving pharmaceutical misuse and abuse by older adults. 2010: Rockville, Md.
11. National institute on drug abuse. N/DA lnfoFacts: prescription and over-the-counter medications. 2009 June [cited 2011 25 Feb.}; Available from: http://www.drugabuse.gov/PDF/Infofacts/PainMed09.pdf.
12. Mastropietro, D. and H. Omidian, Current Approaches in Tamper-Resistant and Abuse-Deterrent Formulations. Drug Development and Industrial Pharmacy, 2012: p. in press.
13. Passik, S. D., et al., Psychiatric and pain characteristics of prescription drug abusers entering drug rehabilitation. J Pain Palliat Care Pharmacother, 2006. 20(2): p. 5-13.
14. McCabe, S. E., et al., Simultaneous and concurrent polydrug use of alcohol and prescription drugs: prevalence, correlates, and consequences. J Stud Alcohol, 2006. 67(4): p. 529-37.
15. U.S. Food and Drug Administration. FDA approves new formulation for OxyContin”. 2010 Apr. 5 [cited 2011 17 Jan.]; Available from: http://www.fda.gov/NewsEvents/Newsroom/PressAnnouncements/ucm207480.htm.
16. Pain Therapeutics Inc. NDA 22-324 Remoxy XRT, advisory committee briefing materials for the anesthetic life support drugs advisory committee meeting of Nov. 13, 2008. 2008 Oct. 12 [cited 2011 17 Mar.]; Available from: http://www.fda.gov/ohrms/dockets/ac/08/briefing/2008-4395b1-02-PAIN.pdf.
17. Pain Therapeutics Inc. FDA complete response letter received for REMOXY. 2011. [cited 2011 20 Mar.]; Available from: http://investor.paintriaIs.com/releasedetail.cfm?Release!D=587311.
18. King Pharmaceuticals Inc. EMBEDAo prescribing information. 2009 June [cited 2011 20 Apr.]; Available from: http://www.kingpharm.com/products/product_document.cfm?brand_name=Embeda&product_spe cific_name=CII&document_type_code=PI.
19. Askari, F., et al., Synthesis and Characterization of Acrylic-based Superabsorbents. Journal of Applied Polymer Science, 1993. 50: p. 1851-1855.
20. Kabiri, K., et al., Synthesis of Fast-Swe/Ung Superabsorbent Hydrogels: Effect of Crosslinker Type and Concentration on Porosity and Absorption Rate. European Polymer Journal, 2003. 39: p. 1341-8.
21. Kabiri, K., H. Omidian, and M. J. Zohuriaan-Mehr, Novel Approach to Highly Porous Superabsorbent Hydrogels: Synergistic Effect of Porogens on Porosity and Swelling Rate. Polymer International, 2003. 52: p. 1158-1164.
22. Omidian, H. and K. Park, Swelling Agents and Devices in Oral Drug Delivery. Journal of Drug Delivery Science and Technology, 2008. 18(2): p. 83-93.
23. Omidian, H., et al., Swelling and Mechanical Properties of Modified HEMA-based Superporous Hydrogels. Journal of Bioactive and Compatible Polymers, 2010. 25(5): p. 483-497.
24. Omidian, H., K. Park, and J. G. Rocca, Recent Developments in Superporous Hydrogels. Journal of Pharmacy and Pharmacology, 2007. 59 (3): p. 317-327.
25. Mastropietro, D. and H. Omidian, Designing Medications that Thwart Abuse, in HPD Reserach Day. 2012: Nova Southeastern University.
26. Omidian, H., et al., Hydrogels Having Enhanced Elasticity and Mechanical Strength Properties, U.S. Pat. No. 6,960,617.
27. Omidian, H. and J. G. Rocca, Formation of Strong Superporous Hydrogels, U.S. Pat. No. 7,056,957 (Issued Jun. 6, 2006).
28. Omidian, H. and J. G. Rocca, Superporous Hydrogels for Heavy Duty Applications, U.S. Pat. No. 7,988,992 (Issued Aug. 2, 2011).
29. Omidian, H., et al., Very-Pure Superporous Hydrogels Having Outstanding Swelling Propertie, US Patent Application 20080206339 (Filed: Feb. 25, 2008—Published: Aug. 28, 2008).
30. (http://www.who.int/substance_abuse/publications/alcohol/en/index.html)
31. H Omidian and J G Rocca., U.S. Pat. No. 7,056,957, Kos Pharmaceuticals, Inc., issued 2006.
32. H Omidian et al., U.S. Pat. No. 6,960,617, Purdue Research Foundation, issued 2005.
33. H Omidian et al., U.S. Pat. No. 7,988,992, Abbott Laboratories, issued 2011.
34. H Omidian et al., US Patent Application 20080206339, Abbott Laboratories, published 2008.
35. H. Omidian and K. Park; Swelling Agents and Devices in Oral Drug Delivery, Journal of Drug Delivery Science and Technology, 18(2), 83-93, 2008
36. National Institute on Alcohol Abuse and Alcoholism (http://www.niaaa.nih.gov/)
37. National Institute on Alcohol Abuse and Alcoholism No. 61, April 2004. Neuroscience Research and
38. Therapeutic Targets
39. World Health Organization; Global Status Report on Alcohol 2004
40. Baum, C., J. P. Hsu, and R. C. Nelson, The Impact of the Addition of Naloxone on the Use and Abuse of Pentazocine. Public Health Reports, 1987. 102(4): p. 426-429.
41. Strain, E. C., J. A. Harrison, and G. E. Bigelow, Induction of opioid-dependent individuals onto buprenorphine and buprenorphine/naloxone soluble-films. Clin Pharmacol Ther, 2011. 89(3): p. 443-9.
42. Farrell, J. J., U.S. Pat. No. 7,968,119, Tamper-proof narcotic delivery system, 2011.
43. Palermo, P. J., R. D. Colucci, and R. F. Kaiko, U.S. Pat. No. 6,228,863, Method of preventing abuse of opioid dosage forms, 2001, Euro-Celtique S.A.
44. Oshlack, B., C. Wright, and J. D. Haddox, U.S. Pat. No. 6,696,088, Tamper-resistant oral opioid agonist formulations, 2004, Euro-Celtique, S.A.
45. Oshlack, B., C. Wright, and J. D. Haddox, U.S. Pat. No. 7,658,939, Tamper-resistant oral opioid agonist formulations, 2010.
46. Oshlack, B., C. Wright, and J. D. Haddox, U.S. Pat. No. 7,718,192, Tamper-resistant oral opioid agonist formulations, 2010, Purdue Pharma L.P.
47. Oshlack, B., C. Wright, and J. D. Haddox, U.S. Pat. No. 7,842,309, Tamper-resistant oral opioid agonist formulations, 2010, Purdue Pharma L.P.
48. Oshlack, B., C. Wright, and J. D. Haddox, U.S. Pat. No. 7,842,311, Tamper-resistant oral opioid agonist formulations, 2010, Purdue Pharma L.P.
49. Breder, C., C. Wright, and B. Oshlack, U.S. Pat. No. 7,914,818, Opioid agonist formulations with releasable and sequestered antagonist, 2011, Purdue Pharma L.P.
50. Shaw, I. F. and J. Berk, U.S. Pat. No. 3,980,766, Orally administered drug composition for therapy in the treatment of narcotic drug addition, 1976, West Laboratories, Inc.
51. Hoffmeister, F., et al., U.S. Pat. No. 4,070,494, Enteral Pharmaceutical Compositions, 1978, Bayer Aktiengesellschaft.
52. Bastin, R. J. and B. H. Lithgow, U.S. Pat. No. 6,309,668, Abuse resistant tablets, 2001, Aventis Pharma Limited.
53. Kumar, V., et al., U.S. Pat. No. 7,201,920, Methods and compositions for deterring abuse of opioid containing dosage forms, 2007, Acura Pharmaceuticals, Inc.
54. Kumar, V. i., et al., U.S. Pat. No. 7,476,402, Methods and compositions for deterring abuse of opioid containing dosage forms, 2009, Acura Pharmaceuticals, Inc.
55. Kumar, V., et al., U.S. Pat. No. 7,510,726, Methods and compositions for deterring abuse of opioid containing dosage forms, 2009, 2009, Acura Pharmaceuticals, Inc.
56. Porter, G., U.S. Pat. No. 4,175,119, Composition and method to prevent accidental and intentional overdosage with psychoactive drugs, 1979.
57. Porter, G., U.S. Pat. No. 4,459,278, Composition and method of immobilizing emetics and method of treating human beings with emetics, 1984, Clear Lake Development Group.
58. Ruan, X., et al., Acute opioid withdrawal precipitated by ingestion of crushed embeda (morphine extended release with sequestered naltrexone): case report and the focused review of the literature. J Opioid Manag, 2010. 6(4): p. 300-3.
59. Passik, S. D., Issues in Long-term Opioid Therapy: Unmet Needs, Risks, and Solutions. Mayo Clinic Proceedings, 2009. 84(7): p. 593-601.
60. Mastropietro, D. J. and H. Omidian, Current approaches in tamper-resistant and abuse-deterrent formulations. Drug Development and Industrial Pharmacy, 2013. 39(5): p. 611-24.
61. Mastropietro, D. J. and H. Omidian, Commercial Abuse-Deterrent Dosage Forms: Clinical Status. Journal of Developing Drugs, 2013. 2(103): p. 1000103.
62. Omidian, H. and D. J. Mastropietro, Fighting a New Drug Epidemic. Journal of Developing Drugs, 2013. 2(1): p. 1000e120.
63. Mohamad Nor, N., et al., Synthesis of activated carbon from lignocellulosic biomass and its applications in air pollution control—a review. Journal of Environmental Chemical Engineering, 2013. 1(4): p. 658-666.
64. National Organic Standards Board Technical Advisory Panel Review, O., Activated Carbon Processing, NOSB TAP Review Compiled by OMRI for the USDA
National Organic Program. 2002 [cited 2013 Dec. 17]; Available from: http://www.ams.usda.gov/AMSv1.0/getfile?dDocName=STELPRDC5066960&acct=nosb.
65. Position statement and practice guidelines on the use of multi-dose activated charcoal in the treatment of acute poisoning. American Academy of Clinical Toxicology; European Association of Poisons Centres and Clinical Toxicologists. J Toxicol Clin Toxicol, 1999. 37(6): p. 731-51.
66. Olson, K. R., Activated charcoal for acute poisoning: one toxicologist's journey. J Med Toxicol, 2010. 6(2): p. 190-8.
67. Potter, T., C. Ellis, and M. Levitt, Activated charcoal: in vivo and in vitro studies of effect on gas formation. Gastroenterology, 1985. 88(3): p. 620-4.
68. Hall, R. G., Jr., H. Thompson, and A. Strother, Effects of orally administered activated charcoal on intestinal gas. Am J Gastroenterol, 1981. 75(3): p. 192-6.
69. Kaaja, R. J., et al., Treatment of cholestasis of pregnancy with peroral activated charcoal. A preliminary study. Scand J Gastroenterol, 1994. 29(2): p. 178-81.
70. Derlet, R. W. and T. E. Albertson, Activated charcoal—past, present and future. West J Med, 1986. 145(4): p. 493-6.
71. Bond, G. R., The role of activated charcoal and gastric emptying in gastrointestinal decontamination: a state-of-the-art review. Ann Emerg Med, 2002. 39(3): p. 273-86.
72. Chyka, P. A., et al., Position paper: Single-dose activated charcoal. Clin Toxicol (Phila), 2005. 43(2): p. 61-87.
73. Wang, X., et al., Effect of Activated Charcoal on Apixaban Pharmacokinetics in Healthy Subjects. Am J Cardiovasc Drugs, 2013.
74. Centers for Disease Control and Prevention. Opioids drive continued increase in drug overdose deaths 2013; Available from: http://www.cdc.gov/media/releases/2013/p0220_drug_overdose_deaths.html.
75. Khosrojerdi, H., R. Afshari, and O. Mehrpour, Should activated charcoal be given after Tramadol overdose? DARU Journal of Pharmaceutical Sciences, 2013. 21(1): p. 1-2.
76. Raffa, R. B., et al., Determination of the adsorption of Tramadol hydrochloride by activated charcoal in vitro and in vivo. Journal of Pharmacological and Toxicological Methods, 2000. 43(3): p. 205-210.
77. Oshlack, B., C. Wright, and J. D. Haddox, Tamper-resistant oral opioid agonist formulations. 2004, Euro-Celtique, S.A.
78. Oshlack, B., C. Wright, and J. D. Haddox, Tamper-resistant oral opioid agonist formulations. 2010, Purdue Pharma L.P.
79. Breder, C., C. Wright, and B. Oshlack, Opioid agonist formulations with releasable and sequestered antagonist. 2011, Purdue Pharma L.P.
80. Bastin, R. J. and B. H. Lithgow, Abuse resistant tablets. 2001, Aventis Pharma Limited.
81. Kumar, V., et al., Methods and compositions for deterring abuse of opioid containing dosage forms. 2007, Acura Pharmaceuticals, Inc.
82. Kumar, V., et al., Methods and compositions for deterring abuse of opioid containing dosage forms. 2009, Acura Pharmaceuticals, Inc.
83. Porter, G., Composition and method to prevent accidental and intentional overdosage with psychoactive drugs. 1979.
84. Porter, G., Composition and method of immobilizing emetics and method of treating human beings with emetics. 1984, Clear Lake Development Group.
85. Omidian H, Mastropietro D. Abuse-Deterrent Pharmaceutical Compositions. Provisional U.S. Patent Application 61/875,173 (Filed Sep. 9, 2013): Nova Southeastern University, Florida, USA (Assignee). 2013.
86. Omidian H, Mastropietro D. A Therapeutic Composition for Alcohol Cessation and Abuse. Provisional U.S. Patent Application 61/918,879 (Filed Dec. 20, 2013): Nova Southeastern University, Florida, USA (Assignee). 2013.
87. Omidian H, Mastropietro D. Abuse Deterrents in Pharmaceutical Compositions. Provisional U.S. Patent Application 61/918,870 (Filed Dec. 20, 2013): Nova Southeastern University, Florida, USA (Assignee). 2013.
88. Omidian H, Mastropietro D. Powerful Deterrent Agents for Abusable Medications. Provisional U.S. Patent Application 61/918,880 (Filed Dec. 20, 2013): Nova Southeastern University, Florida, USA (Assignee). 2013.
89. Mastropietro D, Omidian H. An Aversive Super-Deterrent Agent for Abusable Medications. Provisional U.S. Patent Application 61/919,443 (Filed Dec. 20, 2013): Nova Southeastern University, Florida, USA (Assignee). 2013.
90. Omidian H, Muppalaneni S, Mastropietro D. A Crush-Resistant Vehicle for Abuse-Deterrent Compositions. Invention Disclosure NSU 14/02: Filed with the NSU's Office of Reserach and Technology Transfer 2014.
91. Omidian H, Muppalaneni S. Multifunction Deterrents For Abusable Medications. Invention Disclosure NSU 14/01: Filed with the NSU's Office of Research and Technology Transfer. 2014.

What is claimed is:

1. A therapeutic dosage form, comprising:
one or more pharmaceutically active ingredients;
one or more crosslinked polyacids; and
one or more linear polyacids.

2. The dosage form of claim 123, wherein the weak base is selected from the group consisting a salt of: organic acids, inorganic acids, hydrochloric acid, hydrosulfuric acid, hydrophosphoric acid, and tartaric acid.

3. The dosage form of claim 1, wherein the crosslinked polyacid is insoluble in water.

4. The dosage form of claim 1, wherein the crosslinked polyacid is made using at least one internal hydrolytic process, irradiative process, thermal process, addition of a bi-chemical crosslinker, addition of polyfunctional chemical crosslinker.

5. The dosage form of claim 1, wherein the crosslinked polyacid possess sufficient binding sites to form a stable complex with the one or more pharmaceutically active ingredients.

6. The dosage form of claim 1, wherein the crosslinked polyacid is selected from the group consisting of sodium carboxymethylcellulose, sodium carboxymethylstarch, alginic acid salt, polyacrylate salt, polymethacrylate salt, poly(potassium sulfopropyl acrylate), poly(2-acrylamido 2-methyl1-propane sulfonic acid (AMPS).

7. The dosage form of claim 6, wherein the polyacid is at least one of internally crosslinked or chemically crosslinked.

8. The dosage form of claim 6, wherein the salt is one of sodium, potassium, and ammonium.

9. The dosage form of claim 1, wherein the dosage form comprises one or more crosslinked polyacids, at a polyacid to pharmaceutically active ingredient weight ratio of about 0.1 to about 500.

10. The dosage form of claim 1, wherein the one or more linear polyacids is soluble in water.

11. The dosage form of claim 1, wherein the linear polyacid possesses sufficient binding sites to form a stable complex with the one or more pharmaceutically active ingredients.

12. The dosage form of claim 1, wherein the linear polyacid is selected from the group of water soluble polymers comprising salts of: carboxymethylcellulose, carboxymethylstarch, alginic acid, polyacrylic acid, polymethacrylic acid, poly(sulfopropyl acrylate), and poly(2-acrylamido 2-methyl1-propane sulfonic acid (AMPS).

13. The dosage form of claim 12, wherein the salts are mono-valent.

14. The dosage form of claim 12, wherein the salt is one of sodium, potassium, and ammonium

15. The dosage form of claim 1, wherein the one or more linear polyacids is sodium carboxymethylcellulose.

16. The dosage form of claim 1, wherein the dosage comprises 1-99 wt % of the one or more linear polyacids.

17. The dosage form of claim 1, wherein the one or more pharmaceutically active ingredients, one or more crosslinked polyacids, and one or more linear polyacids are compressed into a tablet along with other tablet excipients.

18. The dosage form of claim 1, further including one or more tablet excipients, and wherein a tablet is formed by:
mixing an aqueous solution of the one or more pharmaceutically active ingredients, the one or more linear polymers, and the one or more crosslinked polyacids;
drying the mix; and
compressing the dried mix along with the one or more tablet excipients.

19. The dosage form of claim 1, wherein the one or more pharmaceutically active ingredients is a weak acid supplied as a salt.

20. The dosage form of claim 1 or 19, further including at least one of a crosslinked polybase and a linear polybase.

21. A therapeutic dosage form, comprising:
one or more pharmaceutically active ingredients;
one or more inorganic clays
a) with binding sites sufficient to form a stable complex with the one or more pharmaceutically active ingredients, when the clay is exposed to the one or more pharmaceutically active ingredients when the dosage form is crushed or subjected to non-physiological tampering conditions, and
b) the clay is physically separated from contact with the one or more pharmaceutically active ingredients before the dosage is orally administered.

22. The dosage form of claim 21, wherein the clay is coated with a coating agent to physically separate the clay from contact with the one or more pharmaceutically active ingredients before the dosage is administered.

23. The dosage form of claim 21, wherein the clay is coated with a water-insoluble coating material.

24. The dosage form of claim 21, wherein the inorganic clay is selected from the group consisting: phyllosilicates; halloysite; kaolinite; illite; montmorillonite; vermiculite; talc; palygorskite; pyrophyllite; zeolite; zeolite made of aluminum silicate sheets; zeolite made of aluminum silicate sheets containing other cations.

25. The dosage form of claim 21, wherein the inorganic clay is bentonite.

26. The dosage form of claim 21, wherein the clay is an aggregate produced using at least one of conventional wet granulation and hot melt extrusion techniques.

27. The dosage form of claim 21, wherein the clay is an aggregate including at least one of a water-soluble or water-dispersible polymer selected from one or more of the group consisting of synthetic polymer, polyacrylic acid, polyvinyl alcohol, polyethylene glycol, polyvinyl pyrrolidone, polyethylene oxide, hydrocolloid gum, alginic acid and its salts, chitosan, carrageenan, gum Arabic, guar gum, agar agar, gelatin, xanthan, locust bean gum, cellulosic, methyl cellulose, carboxymethyl cellulose, hydroxyethyl cellulose, hydroxypropyl cellulose, hydroxypropyl methylcellulose, starch.

28. The dosage form of claim 21, wherein the clay is an aggregate including a polymer, the aggregate bound with hydroxypropyl methylcellulose.

29. The dosage form of claim 22, wherein the coating agent is selected from one or more of the group consisting of water-insoluble polymer, ethyl cellulose, cellulose acetate, cellulose acetate phthalate, cellulose acetate butyrate, shellac, methacrylate copolymer, acrylate copolymer, enteric acrylate copolymer, non-enteric acrylate copolymer, poly(lactic acid), poly(lactide-co-glycolide), hydroxypropyl methylcellulose phthalate, hydroxypropyl methylcellulose acetate succinate, polyvinyl acetate.

30. The dosage form of claim 22, wherein the coating agent is a methacrylic acid ethyl acrylate copolymer.

31. The dosage form of claim 30, wherein one of the solid or the dispersion form of methacrylic acid ethyl acrylate copolymer is used.

32. The dosage form of claim 22, wherein the coating agent is selected from one or more of the group consisting of: animal wax, beeswax, plant wax, carnauba wax, petroleum wax, paraffin, polyethylene wax, stearic acid, magnesium stearate.

33. The dosage form of claim 21, wherein the clay has the form of particles or aggregates, and the dosage form comprises clay particles or aggregates to pharmaceutically active ingredient weight ratio of about 0.1 to about 500.

34. The dosage form of claim 21, wherein the clay has the form of coated particles or aggregates, and the one or more pharmaceutically active ingredients and coated clay are mixed and compressed into a tablet.

35. The dosage form of claim 21, where the dosage form is a tablet formed as a plurality of layers, wherein the clay is in a different layer than the one or more pharmaceutically active ingredient.

36. The dosage form of claim 21, wherein the clay has the form of coated particles or aggregates, and is coated in a continuous extrusion process.

37. The dosage form of claim 22, wherein the dosage form is a capsule, and wherein the one or more pharmaceutically active ingredient is wet granulated, and then incorporated into the capsule along with the coated clay.

38. A therapeutic dosage form, comprising:
one or more pharmaceutically active ingredients, and
at least one of activated carbon or activated porous non-carbon material adsorbent to the one or more pharmaceutically active ingredients and having sufficient adsorption sites to accommodate substantially all of the one or more pharmaceutically active ingredients; and
a physical separation between the at least one of activated carbon or activated porous non-carbon material and the one or more pharmaceutically active ingredients within the dosage form, the at least one of activated carbon or activated porous non-carbon material contactable with the one or more pharmaceutically active ingredients to adsorb the one or more pharmaceutically active ingredients when the physical separation is removed prior to administration of the dosage form.

39. The dosage form of claim 38, wherein the physical separation is a coating about the at least one of activated carbon or activated porous non-carbon material.

40. The dosage form of claim 39, wherein the coating is polymeric.

41. The dosage form of claim 39, wherein the at least one of activated carbon or activated porous non-carbon material is modified via grafting to another substrate configured to enhance an adsorption property of the at least one of activated carbon or activated non-carbon material.

42. The dosage form of claim 41, wherein the substrate enhances the adsorption by at least one of chemical or mechanical interaction with the at least one of activated carbon or activated porous non-carbon material.

43. The dosage form of claim 38, wherein the activated carbon material is at least one of an activated charcoal or medicinal carbon.

44. The dosage form of claim 38, wherein at least one of activated carbon or activated porous non-carbon material has the form of fine particles or aggregates.

45. The dosage form of claim 38, wherein the at least one of activated carbon or activated porous non-carbon material is coated with a water-insoluble coating material.

46. The dosage form of claim 38, wherein the activated porous non-carbon material is an activated silica or activated alumina.

47. The dosage form of claim 38, wherein the at least one of activated carbon or activated porous non-carbon material are produced as aggregates using at least one of conventional wet granulation or hot melt extrusion techniques.

48. The dosage form of claim 38, wherein the at least one of activated carbon or activated porous non-carbon material is formed as an aggregate using a binder selected from the group consisting of at least one of: water-soluble polymer, water-dispersible polymer, synthetic polymer, polyacrylic acid, polyvinyl alcohol, polyethylene glycol, polyvinyl pyrrolidone, polyethylene oxide, hydrocolloid gum, alginic acid and its salts, chitosan, carrageenan, gum Arabic, guar gum, agar agar, gelatin, xanthan, locust bean gum, cellulosic material, methyl cellulose, carboxymethyl cellulose, hydroxyethyl cellulose, hydroxypropyl cellulose, hydroxypropyl methylcellulose, and starch.

49. The dosage form of claim 44, wherein a binder for making the aggregate is hydroxypropyl methylcellulose.

50. The dosage form of claim 44, wherein the particles or aggregates are coated with a material selected from the group consisting of at least one of: water-insoluble polymer, ethyl cellulose, cellulose acetate, cellulose acetate phthalate, cellulose acetate butyrate, shellac, methacrylate copolymer, acrylate copolymer, poly(lactic acid), poly(lactide-co-glycolide), hydroxypropyl methylcellulose phthalate, hydroxypropyl methylcellulose acetate succinate, and polyvinyl acetate.

51. The dosage form of claim 39, wherein the coating is methacrylic acid ethyl acrylate copolymer.

52. The dosage form of claim 51, wherein at least one of the solid or the dispersion form of the methacrylic acid ethyl acrylate copolymer is used.

53. The dosage form of claim 39, wherein the coating is selected from a group consisting of at least one of: animal wax, beeswax, plant wax, carnauba wax, petroleum wax, paraffin, polyethylene wax, water-insoluble wax, stearic acid, and magnesium stearate.

54. The dosage form of claim 38, wherein the at least one of activated carbon or activated porous non-carbon material comprises 1-99 wt % of the dosage form.

55. The dosage form of claim 38, wherein the at least one of activated carbon or activated porous non-carbon material is formed and the one or more pharmaceutically active ingredients are physically mixed and compressed into a tablet along with other tablet excipients.

56. The dosage form of claim 55, where the dosage form is a multi-layer tablet, wherein the at least one of activated carbon or activated porous non-carbon material is separated from the drug layer within the tablet.

57. The dosage form of claim 38, wherein:
the one or more pharmaceutically active ingredients is wet granulated;
the at least one of activated carbon or activated porous non-carbon material is wet granulated separately from the wet granulated pharmaceutically active ingredients;
the wet granulated activated carbon or activated porous non-carbon material is coated with a water insoluble material; and
the wet granulated pharmaceutically active ingredients and the wet granulated and coated activated carbon or activated porous non-carbon material are incorporated into a capsule.

58. A therapeutic dosage form, comprising:
one or more pharmaceutically active ingredients;
one or more organic binding agents;
one or more inorganic binding agents; and
one or more adsorbents.

59. The dosage form of claim 58, wherein the one or more organic binding agent is capable of binding to positively charged pharmaceutically active ingredients.

60. The dosage form of claim 59, wherein the one or more organic binding agent is at least one crosslinked anionic hydrophilic polymer.

61. The dosage form of claim 60, wherein the at least one crosslinked anionic hydrophilic polymer is crosslinked carboxymethylcellulose.

62. The dosage form of claim 61, wherein the one or more organic binding agent is used at a concentration greater than 60% to maximize trapping of the one or more pharmaceutically active ingredients in water, saline, and hydroalcohols, while allowing release of the one or more pharmaceutically active ingredients in 0.1N HCl.

63. The dosage form of claim 61, wherein the one or more organic binding agent is used at 100% concentration to maximum release of the one or more pharmaceutically active ingredients in 0.1N HCl.

64. The dosage form of claim 58, wherein the one or more inorganic binding agent is capable of binding to positively charged pharmaceutically active ingredients.

65. The dosage form of claim 64, wherein the one or more inorganic binding agent is a clay material.

66. The dosage form of claim 65, wherein the clay material is calcium or sodium bentonite.

67. The dosage form of claim 65, wherein the clay material is used at a concentration between about 50% and about 100% to maximum trapping of the one or more pharmaceutically active ingredients in water, saline, aqueous ethyl alcohol, and acidic solutions.

68. The dosage form of claim 65, wherein the clay material is used at 100% concentration to maximum trapping of the one or more pharmaceutically active ingredients in hydroalcoholic solutions.

69. The dosage form of claim 58, wherein the one or more adsorbents has a porous structure capable of adsorbing the one or more pharmaceutically active ingredients.

70. The dosage form of claim 69, wherein the one or more adsorbents is silica or charcoal.

71. The dosage form of claim 70, wherein the one or more adsorbents is medicinal charcoal.

72. The dosage form of claim 69, wherein the one or more adsorbents is used at a concentration between about 0% and about 80% to maximum trapping of the one or more pharmaceutically active ingredients in water, saline, and hydroalcohols but allows release of the one or more pharmaceutically active ingredients in 0.1N HCl.

73. The dosage form of claim 69, wherein the one or more adsorbents is used at 100% concentration to maximum trapping of the one or more pharmaceutically active ingredients in 0.1N HCl.

74. The dosage form of claim 58, wherein the one or more pharmaceutically active ingredients is trapped from solution in water, saline, hydroalcoholic solutions, and acidic solutions.

75. The dosage form of claim 58, wherein the one or more pharmaceutically active ingredients is trapped from solution in water, saline, EtOH 40%, and a pH3 solution, but is released in 0.1N HCl.

76. The dosage form of claim 58, wherein the one or more organic binding agents is crosslinked sodium carboxymethylcellulose; the one or more inorganic binding agents is bentonite; and the one or more adsorbents is charcoal.

77. The dosage form of claim 76, wherein at least one of crosslinked sodium carboxymethylcellulose, bentonite, and charcoal is coated.

78. The dosage form of claim 77, wherein each of crosslinked sodium carboxymethylcellulose, bentonite, and charcoal is coated.

79. The dosage form of claim 76, wherein none of crosslinked sodium carboxymethylcellulose, bentonite, and charcoal are coated.

80. A therapeutic dosage form, comprising:
one or more pharmaceutical active ingredients;
a water-swellable superabsorbent polymer, and
a plastic agent consisting of a thermoplastic water-soluble or water-insoluble polymer which provides mechanical strength to the structure of the dosage form.

81. The dosage form of claim 80, wherein the superabsorbent polymer absorbs at least 40 g/g of deionized water at room temperature.

82. The dosage form of claim 80, wherein the superabsorbent polymer is selected from a group consisting of: chemically-crosslinked homopolymers, copolymers or terpolymers of water-soluble monomers of sodium acrylate, potassium acrylate, sodium methacrylate, potassium methacrylate, potassium sulfopropyl acrylate, acrylamide, 2-acrylamido 2-methyl 1-propane sulfonic acid, and methacrylamidopropyltrimethyl ammonium chloride.

83. The dosage form of claim 80, wherein the superabsorbent polymer comprises 1-99 wt % of the dosage form.

84. The dosage form of claim 80, wherein the superabsorbent polymer comprises 15-25 wt % of the dosage form.

85. The dosage form of claim 80, wherein the plastic agent is a polymer with a glass transition temperature between about 40° C. and about 100° C.

86. The dosage form of claim 80, wherein the plastic agent is a polymer with a glass transition temperature between about 40° C. and about 55° C.

87. The dosage form of claim 80, wherein the plastic agent is at least one of a low glass transition homopolymers of vinyl acetate and a low glass transition copolymer of vinyl acetate.

88. The dosage form of claim 80 wherein the plastic agent comprises 1-99 wt % of the dosage form.

89. The dosage form of claim 80 wherein the plastic agent comprises 15-25 wt % of the dosage form.

90. The dosage form of claim 80, further including a superviscosifier selected from the group consisting of: water soluble polymer, polyethylene oxide, methyl cellulose, hydroxypropyl methylcellulose, carboxymethyl cellulose, hydroxyethyl cellulose, hydroxypropyl cellulose, xanthan gum, guar gum, and non-crosslinked forms of the polymers of claim 82.

91. The dosage form of claim 80 further including a very high molecular weight polyethylene oxide superviscosifier.

92. The dosage form of claim 80, further including a polyethylene oxide superviscosifier with molecular weight equal or greater than 5,000,000 Da.

93. The dosage form of claim 80, wherein the superabsorbent polymer is crosslinked poly(sodium acrylate), and the plastic agent is a physical blend of poly(vinyl acetate) and poly(vinyl pyrrolidone).

94. The dosage form of claim 93, wherein the plastic agent is Kollidone SR® (BASF).

95. The dosage form of claim 90 wherein the superabsorbent polymer is crosslinked polyacrylamide, and the plastic agent is a physical blend of poly(vinyl acetate) and poly(vinyl pyrrolidone).

96. The dosage form of claim 80 wherein the superabsorbent polymer is crosslinked poly(sulfopropyl acrylate potassium), and the plastic agent is a physical blend of poly(vinyl acetate) and poly(vinyl pyrrolidone).

97. The dosage form of claim 80 wherein the superabsorbent polymer is crosslinked poly(2-acrylamido-propane sulfonic acid), and the plastic agent is a physical blend of poly(vinyl acetate) and poly(vinyl pyrrolidone).

98. The dosage form of claim 80, further including polyethylene oxide as a superviscosifying polymer.

99. The dosage form of claim 80, formed by heat-treating the dosage form at a temperature above the glass transition temperature of the plastic agent.

100. A therapeutic dosage form, comprising one or more superabsorbent polymers operative to absorb significantly more alcohol than the weight of the superabsorbent polymer.

101. A method of at least one of treating acute alcohol intoxication, treating alcohol abuse, and promoting alcohol cessation, comprises providing a dosage form including a superabsorbent polymer operative to absorb alcohol.

102. The dosage form of claim 100, wherein the superabsorbent polymer swells in deionized water from about 100 g/g to about 1000 g/g.

103. The dosage form of claim 100, wherein the superabsorbent polymer swells in deionized water from about 300 g/g to about 600 g/g within 15 minute swelling time under mixing at room temperature.

104. The dosage form of claim 100, wherein the superabsorbent polymer is selected from the group consisting of: chemically-crosslinked homopolymers, copolymers or terpolymers of water-soluble and alcohol-soluble monomers of acrylic acid and its salts, methacrylic acid and its salts, sulfopropyl acrylic acid and its salts, acrylamide, 2-acrylamido 2-methyl 1-propane sulfonic acid, and methacrylamidopropyltrimethyl ammonium chloride.

105. The dosage form of claim 100, wherein the superabsorbent polymer is at least one of an acrylamide based homopolymer, acrylamide based copolymer, or acrylamide based terpolymer.

106. The dosage form of claim 100, wherein the superabsorbent polymer is chemically crosslinked polyacrylamide.

107. The dosage form of claim 100, wherein the superabsorbent polymer comprises 1 to 100 wt % of the composition.

108. The dosage form of claim 100, further comprising a superviscosifier selected from the group consisting of water soluble polymers with high affinity for alcohol: polyethylene oxide, methyl cellulose, hydroxypropyl methylcellulose, carboxymethyl cellulose, hydroxyethyl cellulose, hydroxypropyl cellulose, xanthan gum, guar gum, and the non-crosslinked polymers of claim 104.

109. The dosage form of claim 100, wherein the superviscosifier is very high molecular weight polyethylene oxide.

110. The dosage form of claim 100, wherein the superviscosifier is polyethylene oxide at molecular weights equal or greater than 5,000,000 Da.

111. The dosage form of claim 109, wherein the Cone & Plate shear viscosity of a 2 w/v % solution of the superviscosifier in water at 22-24° C. and a shear rate of 2 sec⁻¹ is from about 4700 to about 11,100 cP.

112. The dosage form of claim 111, wherein the viscosity at shear rate of 2 sec⁻¹ is from about 7100 to about 8700 cP.

113. The dosage form of claim 108, further including 1-99 wt % of the superviscosifier.

114. The dosage form of claim 108, comprising 50-99% of superabsorbent and 1-50% of the superviscosifier, when the hydroalcoholic solution contains less than 40% ethanol.

115. The dosage form of claim 108, comprising 1-50% of superabsorbent and 50-99% of the superviscosifier, when the hydroalcoholic solution contains greater than 40% of ethanol.

116. The dosage form of claim 100, wherein the superabsorbent polymer is crosslinked polyacrylamide and the superviscosifier is polyethylene oxide.

117. The dosage form of claim 100, wherein the superabsorbent polymer is crosslinked poly (2-acrylamido-propane sulfonic acid), and the superviscosifier is polyethylene oxide.

118. The dosage form of claim 100, wherein the dosage form is formed as one of a tablet, capsule, gel, or patch.

119. The dosage form of claim 100, further including a pharmaceutically active ingredient.

120. The dosage form of claims 1, 21, 38, 58, or 80, further including at least one of tablet excipients for tableting, capsule excipients for encapsulation, and patch excipients for transdermal patches.

121. The dosage form of claims 1, 21, 38, or 80, wherein the one or more pharmaceutically active ingredients treats an illness selected from the group consisting: anxiety, depression, sleep disorders, pain, lack of energy, attention deficit, cough, and cold.

122. The dosage form of claims 1, 21, 38, 58, or 80, wherein the one or more pharmaceutically active ingredients is selected from the group of barbiturates comprising: phenobarbitals, benzodiazepines, codeine, morphine, oxycodone, oxymorphone, hydrocodone, hydromorphone, Tramadol, amphetamines, methyl phenidate, dextromethorphan, and pseudoephedrine.

123. The dosage form of claims 1, 21, or 38, wherein the one or more pharmaceutically active ingredients is in the form of its weak base.

124. The dosage form of claims 1, 21, 38, or 58 wherein the dosage form is a tablet.

125. The dosage form of claims 1, 21, 38, or 58, wherein the dosage form is a capsule.

126. The dosage form of claims 1, 21, 38, or 58, wherein the dosage has a form selected from the group consisting of gel, suppository, suspension, emulsion, micro sized dispersion, nano-sized dispersion, semi-solid, paste, ointment, lozenge, strip, film, and rod.

127. The dosage form of claim 1, wherein the dosage form comprises one or more crosslinked polyacids, at a polyacid to pharmaceutically active ingredient weight ratio of about 1 to about 50.

128. The dosage form of claim 21, wherein the clay has the form of particles or aggregates, and the dosage form comprises clay particles or aggregates to pharmaceutically active ingredient weight ratio of about 1 to about 50.

129. The dosage form of claim 109, wherein the Cone & Plate shear viscosity of the 2 w/v % solution of the superviscosifier in 20 v/v % ethanol in water at 22-24° C. and shear rate of 2 sec⁻¹ is from about 5200 to about 12000 cP.

130. The dosage form of claim 100, wherein the Cone & Plate shear viscosity of the 2 w/v % solution of the superviscosifier in 20 v/v % ethanol in water at 22-24° C. and shear rate of 2 sec⁻¹ is advantageously from about 7800 to about 9600 cP.

131. The dosage form of claim 109, wherein the Cone & Plate shear viscosity of the 2 wt % solution of the superviscosifier in 60 v/v % ethanol in water at 22-24° C. and shear rate of 20 sec⁻¹ is from about 1200 to about 3000 cP.

132. The dosage form of claim 100, wherein the Cone & Plate shear viscosity of the 2 wt % solution of the superviscosifier in 60 v/v % ethanol in water at 22-24° C. and shear rate of 20 sec⁻¹ is advantageously from about 1900 to about 2300 cP.
